(19) United States
(12) Patent Application Publication
(10) Pub. No.: US 2025/0161382 A1
(43) Pub. Date: May 22, 2025

(54) COMPOSITIONS FOR GUT HEALTH

(71) Applicant: INTERNATIONAL N&H DENMARK APS, Kongens Lyngby (DK)

(72) Inventors: John Thomas Gannon, Hockessin, DE (US); Michael W. Bostick, Wilmington, DE (US); Amanda Chan, Townsend, DE (US); Raymond E. Jackson, Newark, DE (US); Victoria Vallejo Kelly, Palo Alto, CA (US); Alexander D. Kopatsis, Wilmington, DE (US); Claus Lang, Palo Alto, CA (US); Qiong Wang, Wilmington, DE (US); Julia Yager, Wilmington, DE (US); Rick W. Ye, Wilmington, DE (US)

(21) Appl. No.: 19/029,860

(22) Filed: Jan. 17, 2025

Related U.S. Application Data
(62) Division of application No. 17/770,042, filed on Apr. 18, 2022, now Pat. No. 12,239,675.
(60) Provisional application No. 62/923,781, filed on Oct. 21, 2019.

(51) Int. Cl. A61K 35/747; A23K 10/18; A23K 20/189; A23K 50/75; A61K 9/00; A61K 35/741; A61K 35/742; A61K 38/46; A61K 38/47; A61K 38/48; A61P 31/04

(52) U.S. Cl. CPC A61K 35/747; A23K 10/18; A23K 20/189; A23K 50/75; A61K 9/0056; A61K 35/741; A61K 35/742; A61K 38/465; A61K 38/47; A61K 38/48; A61P 31/04; A23V 2400/173; A23V 2400/181

(57) ABSTRACT

Provided herein, inter alia, are compositions of short chain fatty acid (SCFA)-producing microorganisms and methods of making and using the same to inhibit pathogenic bacterial populations in the gastrointestinal tracts of an animal and additionally promote improvement of one or more metrics in an animal, such as increased bodyweight gain, decreased feed conversion ratio (FCR), improved gut barrier integrity, reduced mortality, reduced pathogen infection, and reduced pathogen shedding in feces.

### CROSS-REFERENCE TO RELATED APPLICATIONS

This application is a divisional of U.S. Non-provisional patent application Ser. No. 17/770,042, filed Apr. 18, 2022, which is a U.S. national stage application under 35 U.S.C. § 371 of International Application No. PCT/US2020/055937, filed Oct. 16, 2020, which claims priority to U.S. Provisional Patent Application No. 62/923,781, filed Oct. 21, 2019, the disclosures of each of which is incorporated by reference herein in its entirety.

### INCORPORATION BY REFERENCE OF THE SEQUENCE LISTING

The present application is being filed with a Sequence Listing in electronic format. The Sequence Listing is provided as a file entitled NB41584-US-PCD_SeqList.xml, created on Jan. 16, 2025, which is 40,182 bytes in size. The information in the electronic format of the Sequence Listing is incorporated by reference in its entirety.

### FIELD OF THE INVENTION

Provided herein, inter alia, are multi-strain direct fed microbial bacterial consortia useful for improving animal gut health and/or performance as well as methods of making and using the same.

### BACKGROUND

In monogastric animal species such as birds, the gastrointestinal tract and intestinal-associated microflora are not only involved in digestion and absorption but also interact with the immune and central nervous system to modulate health. The inside of the intestinal tract is coated with a thin layer of sticky, viscous mucous, and embedded in this mucus layer are millions and millions of bacteria and other microbes. When the intestinal bacteria are in balance (i.e., the good bacteria outnumber the bad bacteria), the gut is said to be healthy. A healthy microbiota provides the host with multiple benefits, including colonization resistance to a broad spectrum of pathogens, essential nutrient biosynthesis and absorption, and immune stimulation that maintains a healthy gut epithelium and an appropriately controlled systemic immunity. In settings of “dysbiosis” or disrupted symbiosis, microbiota functions can be lost or deranged, resulting in increased susceptibility to pathogens, altered metabolic profiles, or induction of proinflammatory signals that can result in local or systemic inflammation or autoimmunity. Thus, the intestinal microbiota of poultry plays a significant role in the pathogenesis of many diseases and disorders, including a variety of pathogenic infections of the gut such as coccidiosis or necrotic enteritis.

Over the past several years, there has been increasing governmental and consumer pressure applied to the animal feed industry to decrease or curtail the use of antibiotics as components of animal nutrition feeding regimens. This pressure is due in large part to the recognition that use of such antibiotics contribute to the rise of antibiotic-resistant pathogenic microorganisms. However, this “No Antibiotics Ever” consumer trend, especially in the poultry industry, has led to the re-emergence of bacterial diseases, particularly necrotic enteritis (Poultry Science, Volume 97, Issue 6, 1 Jun. 2018, 1929-1933). Necrotic enteritis is caused by certain toxin-producing Clostridium perfringens strains. Under certain conditions C. perfringens produces toxins which cause lesions in the small intestines and ultimately result in reduced growth or death of the infected birds. Accordingly, there is currently a recognized need for products and methods capable of reducing pathogenic bacterial populations in the digestive tracts of domesticated animals such as birds without the use of traditionally-used antibiotics.

The subject matter disclosed herein addresses these needs and provides additional benefits as well.

### SUMMARY

Provided herein, inter alia, are multi-strain direct fed microbial bacterial consortia of short chain fatty acid (SCFA)-producing microorganisms and methods of making and using the same to inhibit pathogenic bacterial populations in the gastrointestinal tracts of an animal (such as birds, for example, chickens) and additionally promote improvement of one or more metrics in an animal such as increased bodyweight gain, decreased feed conversion ratio (FCR), improved gut barrier integrity, reduced mortality, reduced pathogen infection, and reduced pathogen shedding in feces.

Accordingly, in some aspects, provided herein are feed additive compositions comprising a direct fed microbial (DFM) comprising one or more biologically pure bacterial strains of Anaerotruncus colihominis. In some embodiments, the feed additive composition comprises one, two, three or four strains of A. colihominis. In some embodiments of any of the embodiments disclosed herein, the feed additive composition comprises a) a bacterial strain having a 16S ribosomal RNA sequence displaying at least 97.0% sequence similarity to a 16S ribosomal RNA sequence of a Anaerotruncus colihominis strain W1 deposited at Westerdijk Fungal Biodiversity Institute (WFDB) under number CBS 146120; b) a bacterial strain having a 16S ribosomal RNA sequence displaying at least 97.0% sequence similarity to a 16S ribosomal RNA sequence of a Anaerotruncus colihominis strain W2 deposited at Westerdijk Fungal Biodiversity Institute (WFDB) under number CBS 146122; c) a bacterial strain having a 16S ribosomal RNA sequence displaying at least 97.0% sequence similarity to a 16S ribosomal RNA sequence of a Anaerotruncus colihominis strain W3 deposited at Westerdijk Fungal Biodiversity Institute (WFDB) under number CBS 146123; and d) a bacterial strain having a 16S ribosomal RNA sequence displaying at least 97.0% sequence similarity to a 16S ribosomal RNA sequence of a Anaerotruncus colihominis strain W4 deposited at Westerdijk Fungal Biodiversity Institute (WFDB) under number CBS 146121. In some embodiments of any of the embodiments disclosed herein, the feed additive composition comprises a) A. colihominis strain W1 (CBS 146120) or a live strain having all of the identifying characteristics of A. colihominis strain W1 (CBS 146120); b) A. colihominis strain W2 (CBS 146122) or a live strain having all of the identifying characteristics of A. colihominis strain W2 (CBS 146122); c) A. colihominis strain W3 (CBS 146123) or a live strain having all of the identifying characteristics of A. colihominis strain W3 (CBS 146123); and d) A. colihominis strain W4 (CBS 146121) or a live strain having all of the identifying characteristics of A. colihominis strain W4 (CBS 146121) either (i) alone; or (ii) in combination with a culture supernatant derived from each of these strains. In some embodiments, the feed additive composition further comprises biologically pure bacterial strains of a) a Coprococcus sp.; b) Clostridium lactatifermentans; and c) Pseudoflavonifractor capillosus. In some embodiments, the feed additive composition comprises a) a bacterial strain having a 16S ribosomal RNA sequence displaying at least 97.0% sequence similarity to a 16S ribosomal RNA sequence of a Coprococcus sp. strain M1 deposited at Westerdijk Fungal Biodiversity Institute (WFDB) under number CBS 146125; b) a bacterial strain having a 16S ribosomal RNA sequence displaying at least 97.0% sequence similarity to a 16S ribosomal RNA sequence of a Anaerotruncus colihominis strain M2 deposited at Westerdijk Fungal Biodiversity Institute (WFDB) under number CBS 146119; c) a bacterial strain having a 16S ribosomal RNA sequence displaying at least 97.0% sequence similarity to a 16S ribosomal RNA sequence of a Clostridium lactatifermentans strain M3 deposited at Westerdijk Fungal Biodiversity Institute (WFDB) under number CBS 146124; and d) a bacterial strain having a 16S ribosomal RNA sequence displaying at least 97.0% sequence similarity to a 16S ribosomal RNA sequence of a Pseudoflavonifractor capillosus strain M4. In some embodiments of any of the embodiments disclosed herein, the feed additive composition comprises a) Coprococcus sp. strain M1 (CBS 146125) or a live strain having all of the identifying characteristics of Coprococcus sp. strain M1 (CBS 146125); b) A. colihominis strain M2 (CBS 146119) or a live strain having all of the identifying characteristics of A. colihominis strain M2 (CBS 146119); c) C. lactatifermentans strain M3 (CBS 146124) or a live strain having all of the identifying characteristics of C. lactatifermentans strain M3 (CBS 146124); and d) a P. capillosus strain either (i) alone; or (ii) in combination with a culture supernatant derived from each of these strains.

In other aspects, provided herein is a feed additive composition comprising a direct fed microbial (DFM) comprising one or more biologically pure bacterial strains of a short chain fatty acid (SCFA)-producing bacterial strain and one or more biologically pure lactic acid producing bacterial strains. In some embodiments, the feed additive composition comprises a) a biologically pure strain of Clostridium lactatifermentans; and b) two biologically pure lactic acid producing bacterial strains. In some embodiments of any of the embodiments disclosed herein, the two biologically pure lactic acid producing bacterial strains are Lactobacillus salivarius and Lactobacillus reuteri. In some embodiments of any of the embodiments disclosed herein, the feed additive composition comprises a) a bacterial strain having a 16S ribosomal RNA sequence displaying at least 97.0% sequence similarity to a 16S ribosomal RNA sequence of a Clostridium lactatifermentans strain 2F1 deposited at Westerdijk Fungal Biodiversity Institute (WFDB) under number 146124; b) a bacterial strain having a 16S ribosomal RNA sequence displaying at least 97.0% sequence similarity to a 16S ribosomal RNA sequence of a Lactobacillus salivarius strain 2F2 deposited at Westerdijk Fungal Biodiversity Institute (WFDB) under number CBS 146126; and c) a bacterial strain having a 16S ribosomal RNA sequence displaying at least 97.0% sequence similarity to a 16S ribosomal RNA sequence of a Lactobacillus reuteri strain 2F3 deposited at Westerdijk Fungal Biodiversity Institute (WFDB) under number CBS 145921. In some embodiments of any of the embodiments disclosed herein, the feed additive composition comprises a) C. lactatifermentans strain 2F1 (CBS 146124) or a live strain having all of the identifying characteristics of C. lactatifermentans strain 2F1 (CBS 146124); b) L. salivarius strain 2F2 (CBS 146126) or a live strain having all of the identifying characteristics of L. salivarius strain 2F2 (CBS 146126); and c) L. reuteri strain 2F3 (CBS 145921) or a live strain having all of the identifying characteristics of L. reuteri strain 2F3 (CBS 145921) either (i) alone; or (ii) in combination with a culture supernatant derived from each of these strains. In some embodiments of any of the embodiments disclosed herein a) the 16S ribosomal RNA sequence of Anaerotruncus colihominis strain W1 comprises the nucleotide sequence of SEQ ID NO:1; b) the 16S ribosomal RNA sequence of Anaerotruncus colihominis strain W2 comprises the nucleotide sequence of SEQ ID NO:2; c) the 16S ribosomal RNA sequence of Anaerotruncus colihominis strain W3 comprises the nucleotide sequence of SEQ ID NO:3; and d) the 16S ribosomal RNA sequence of Anaerotruncus colihominis strain W4 comprises the nucleotide sequence of SEQ ID NO:4. In some embodiments of any of the embodiments disclosed herein a) the 16S ribosomal RNA sequence of Coprococcus sp. strain M1 comprises the nucleotide sequence of SEQ ID NO:5; b) the 16S ribosomal RNA sequence of Anaerotruncus colihominis strain M2 comprises the nucleotide sequence of SEQ ID NO:6; c) the 16S ribosomal RNA sequence of Clostridium lactatifermentans strain M3 comprises the nucleotide sequence of SEQ ID NO: 7; and d) the 16S ribosomal RNA sequence of Pseudoflavonifractor capillosus strain M4 comprises the nucleotide sequence of SEQ ID NO:8. In some embodiments of any of the embodiments disclosed herein a) the 16S ribosomal RNA sequence of Clostridium lactatifermentans strain 2F1 comprises the nucleotide sequence of SEQ ID NO:9; b) the 16S ribosomal RNA sequence of Lactobacillus salivarius strain 2F2 comprises the nucleotide sequence of SEQ ID NO:10; and c) the 16S ribosomal RNA sequence of Lactobacillus reuteri strain 2F3 comprises the nucleotide sequence of SEQ ID NO:11. In some embodiments of any of the embodiments disclosed herein, the composition produces one or more short chain fatty acids selected from the group consisting of butyrate, isobutyrate, propionate, acetate, isovalerate, and valerate. In some embodiments of any of the embodiments disclosed herein, the feed additive composition further comprises one or more enzymes. In some embodiments, the one or more enzymes are selected from the group consisting of a phytase, a protease, an amylase, a xylanase and a beta-glucanase. In some embodiments of any of the embodiments disclosed herein, each strain is present at a concentration of at least about 1×10³ CFU/g feed additive composition to at least about 1×10⁹ CFU/g feed additive composition. In some embodiments of any of the embodiments disclosed herein, the composition inhibits at least one pathogen selected from avian pathogenic Salmonella sp., Escherichia coli, Clostridium perfringens and Enterobacteriaceae in a gastrointestinal tract of a bird having ingested an effective amount of said direct fed microbial composition.

In further aspects, provided herein is a premix comprising any of the feed additive compositions disclosed herein and at least one mineral and/or at least one vitamin. In still other aspects, provided herein is a feed or feedstuff comprising any of the feed additive compositions disclosed herein or any of the premixes disclosed herein.

In another aspect, provided herein are kits comprising a) any of the feed additive compositions disclosed herein; and b) written instructions for administration to an animal. In some embodiments, the kit further comprises one or more enzymes. In some embodiments, the one or more enzymes are selected from the group consisting of a phytase, a protease, an amylase, a xylanase and a beta-glucanase.

In further aspects, provided herein is a method for improving one or more metrics in an animal selected from the group consisting of increased bodyweight gain, intestinal health status, decreased feed conversion ratio (FCR), improved gut barrier integrity, reduced mortality, reduced pathogen infection, and reduced pathogen shedding in feces comprising administering an effective amount of any of the feed additive compositions disclosed herein, any of the premixes disclosed herein, or any of the feeds or feedstuffs disclosed herein to the animal, thereby improving the one or more metrics in the animal. In some embodiments, the feed additive composition increases one or more of the acetate, isobutyrate, butyrate, isovalerate, and/or valerate content of the gastrointestinal tract of the animal. In some embodiments of any of the embodiments disclosed herein, the feed additive composition increases the butyrate content of the gastrointestinal tract of the animal. In some embodiments of any of the embodiments disclosed herein, the pathogen is one or more of Clostridium perfringens, Campylobacter jejuni, Enterobacteriaceae, a Salmonela sp., and/or Escherichia coli. In some embodiments of any of the embodiments disclosed herein, the method further treats, prevents, or decreases incidence of necrotic enteritis. In some embodiments of any of the embodiments disclosed herein, the animal is a domesticated bird. In some embodiments, the domesticated bird is selected from the group consisting of chickens, turkeys, ducks, geese, quail, emus, ostriches, and pheasant. In some embodiments, the chicken is a broiler or a layer.

In other aspects, provided herein is a method for preparing a feed additive composition comprising combining a) a bacterial strain having a 16S ribosomal RNA sequence displaying at least 97.0% sequence similarity to a 16S ribosomal RNA sequence of a Anaerotruncus colihominis strain W1 deposited at CBS under number CBS 146120; b) a bacterial strain having a 16S ribosomal RNA sequence displaying at least 97.0% s sequence similarity to a 16S ribosomal RNA sequence of a Anaerotruncus colihominis strain W2 deposited at CBS under number CBS 146122; c) a bacterial strain having a 16S ribosomal RNA sequence displaying at least 97.0% sequence similarity to a 16S ribosomal RNA sequence of a Anaerotruncus colihominis strain W3 deposited at CBS under number CBS 146123; and d) a bacterial strain having a 16S ribosomal RNA sequence displaying at least 97.0% sequence similarity to a 16S ribosomal RNA sequence of a Anaerotruncus colihominis strain W4 deposited at CBS under number CBS 146121. In some embodiments a) the A. colihominis strain W1 is an A. colihominis strain W1 (CBS 146120) or a live strain having all of the identifying characteristics of A. colihominis strain W1 (CBS 146120); b) the A. colihominis strain W2 is an A. colihominis strain W2 (CBS 146122) or a live strain having all of the identifying characteristics of A. colihominis strain W2 (CBS 146122); c) the A. colihominis strain W3 is an A. colihominis strain W3 (CBS 146123) or a live strain having all of the identifying characteristics of A. colihominis strain W3 (CBS 146123); and d) the A. colihominis strain W4 is an A. colihominis strain W4 (CBS 146121) or a live strain having all of the identifying characteristics of A. colihominis strain W4 (CBS 146121) either (i) alone; or (ii) in combination with a culture supernatant derived from each of these strains.

In still other aspects, provided herein is a method for preparing a feed additive composition comprising combining a) a bacterial strain having a 16S ribosomal RNA sequence displaying at least 97.0% sequence similarity to a 16S ribosomal RNA sequence of a Coprococcus sp. strain M1 deposited at CBS under number CBS 146125; b) a bacterial strain having a 16S ribosomal RNA sequence displaying at least 97.0% sequence similarity to a 16S ribosomal RNA sequence of a Anaerotruncus colihominis strain M2 deposited at CBS under number CBS 146119; c) a bacterial strain having a 16S ribosomal RNA sequence displaying at least 97.0% sequence similarity to a 16S ribosomal RNA sequence of a Clostridium lactatifermentans strain M3 deposited at CBS under number CBS 146124; and d) a bacterial strain having a 16S ribosomal RNA sequence displaying at least 97.0% sequence similarity to a 16S ribosomal RNA sequence of a Pseudoflavonifractor capillosus strain M4. In some embodiments, the a) Coprococcus sp. strain M1 is an Coprococcus sp. strain M1 (CBS 146125) or a live strain having all of the identifying characteristics of Coprococcus sp. strain M1 (CBS 146125); b) the A. colihominis strain M2 is an A. colihominis strain M2 (CBS 146119) or a live strain having all of the identifying characteristics of A. colihominis strain M2 (CBS 146119); and c) the C. lactatifermentans strain M3 is a C. lactatifermentans strain M3 (CBS 146124) or a live strain having all of the identifying characteristics of C. lactatifermentans strain M3 (CBS 146124) either (i) alone; or (ii) in combination with a culture supernatant derived from each of these strains.

In still other aspects, provided herein is a method for preparing a feed additive composition comprising combining a) a bacterial strain having a 16S ribosomal RNA sequence displaying at least 97.0% sequence similarity to a 16S ribosomal RNA sequence of a Clostridium lactatifermentans strain 2F1 deposited at CBS under number CBS 146124; b) a bacterial strain having a 16S ribosomal RNA sequence displaying at least 97.0% s sequence similarity to a 16S ribosomal RNA sequence of a Lactobacillus salivarius strain 2F2 deposited at CBS under number CBS 146126; and c) a bacterial strain having a 16S ribosomal RNA sequence displaying at least 97.0% sequence similarity to a 16S ribosomal RNA sequence of a Lactobacillus reuteri strain 2F3 deposited at CBS under number CBS 145921. In some embodiments a) the C. lactatifermentans strain 2F1 is an C. lactatifermentans strain W1 (CBS 146124) or a live strain having all of the identifying characteristics of C. lactatifermentans strain 2F1 (CBS 146124); b) the L. salivarius strain 2F2 is an L. salivarius strain 2F2 (CBS 146126) or a live strain having all of the identifying characteristics of L. salivarius strain 2F2 (CBS 146126); and c) the L. reuteri strain 2F3 is an L. reuteri strain 2F3 (CBS 145921) or a live strain having all of the identifying characteristics of L. reuteri strain 2F3 (CBS 145921) either (i) alone; or (ii) in combination with a culture supernatant derived from each of these strains. In some embodiments of any of the embodiments disclosed herein, the method further comprises combining one or more enzyme with the feed additive composition. In some embodiments, the one or more enzymes are selected from the group consisting of a phytase, a protease, an amylase, a xylanase and a beta-glucanase. In some embodiments of any of the embodiments disclosed herein, at least about 1×10³ CFU/g feed additive composition to at least about 1×10⁹ CFU/g feed additive composition is combined to form the feed additive composition. In some embodiments of any of the embodiments disclosed herein, the method further comprises packaging the feed additive composition.

In another aspect, provided herein is a method for preparing a premix comprising combining any of the feed additive compositions disclosed herein with at least one mineral and/or at least one vitamin. In some embodiments, the method further comprises comprising packaging the premix.

Each of the aspects and embodiments described herein are capable of being used together, unless excluded either explicitly or clearly from the context of the embodiment or aspect.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purposes.

### BRIEF DESCRIPTION OF THE DRAWINGS

FIG. 1A depicts a bar graph showing short chain fatty acid (SCFA) production by Anaerotruncus colihominis in culture at 24 and 48 hours. FIG. 1B depicts a bar graph showing SCFA production by Clostridium lactatifermentans in culture at 24 and 48 hours.

FIG. 2 depicts a bar graph showing SCFA production by bacteria individually as well as in co-culture. R1=Coprococcus sp. strain M1; Ac=Anaerotruncus colihominis strain M2; Cl=Clostridium lactatifermentans strain M3; and Pc=Pseudoflavonifractor capillosus strain M4.

### DETAILED DESCRIPTION

A variety of microbial species have been shown to have certain degrees of efficacy against gut pathogens either in vitro or in vivo. Commonly studied organisms have included Bacillus or Lactobacillus. Another group of microbes of potential interest are obligate anaerobes that can produce short-chain fatty acids (SCFAs). SCFAs, for example, propionate and butyrate (i.e. the conjugate bases of propionic and butyric acid, respectively), are suspected to have many beneficial properties for gut health (see, e.g., Adv Immunol. 2014; 121:91-119; Eur J Pharmacol. 2018 Jul. 15;831:52-59; Adv Nutr. 2019 Jan. 1;10 (suppl_1): S49-S66).

As described in more detail herein, the inventors have surprisingly discovered that administering specific species of SCFA-producing microbes to animals (such as domesticated birds, for example, chickens) can improve performance on one or more metrics that include increased bodyweight gain, intestinal health status, decreased feed conversion ratio (FCR), improved gut barrier integrity, reduced mortality, reduced pathogen infection (such as, but not limited to, infection by Clostridium perfringens), and reduced pathogen shedding in feces. Without being bound to theory, it is believed that SCFAs play an important role in the prevention of intestinal inflammation and in the maintenance of intestinal homeostasis. While many obligate anaerobes have the capability to produce SCFAs, not all species can provide benefits to animals when administered as a feed additive or as part of a feed. However, as will be described in the Examples section, administration of particular combinations of microbials was discovered to be surprisingly effective in the prevention and/or treatment of gut pathogenesis in animals as well as maintenance of overall health.

### I. Definitions

Short-chain fatty acids (SCFAs) (also referred to as volatile fatty acids (VFAs)), as used herein, are fatty acids with less than six carbon atoms. Non-limiting examples of SCFAs include, formic acid (methanoic acid), acetic acid (ethanoic acid), propionic acid (propanoic acid), butanoic acid (butyric acid), isobutyric acid (2-methylpropanoic acid), valeric acid (pentanoic acid), and isovaleric acid (3-methylbutanoic acid). Inclusive in this definition of SCFAs are also the conjugate bases of SCFAs including, without limitation, formate, acetate, propionate, butyrate, isobutyrate, valerate, and isovalerate.

As used herein, “microorganism” or “microbe” refers to a bacterium, a fungus, a virus, a protozoan, and other microbes or microscopic organisms.

As used here in the term “direct fed microbial” refers to a composition for consumption by animals (i.e. as an or as a component of animal feed) that contains viable microorganisms, i.e. microorganisms that are capable of living and reproducing. See, for example, U.S. Pat. No. 8,420,074. A direct fed microbial may comprise one or more (such as any of 1, 2, 3, 4, 5, or 6 or more) of any of the microbial strains described herein.

A bacterial “strain” as used herein refers to a bacterium which remains genetically unchanged when grown or multiplied. The multiplicity of identical bacteria is included.

By “at least one strain,” is meant a single strain but also mixtures of strains comprising at least two strains of microorganisms. By “a mixture of at least two strains,” is meant a mixture of two, three, four, five, six or even more strains. In some embodiments of a mixture of strains, the proportions can vary from 1% to 99%. When a mixture comprises more than two strains, the strains can be present in substantially equal proportions in the mixture or in different proportions.

For purposes of this disclosure, a “biologically pure strain” means a strain containing no other bacterial strains in quantities sufficient to interfere with replication of the strain or to be detectable by normal bacteriological techniques. “Isolated” when used in connection with the organisms and cultures described herein includes not only a biologically pure strain, but also any culture of organisms which is grown or maintained other than as it is found in nature. In some embodiments, the strains are mutants, variants, or derivatives of strains W1, W2, W3, W4, M1, M2, M3, M4, 2F1, 2F2 and 2F3 that also provide benefits comparable to that provided by W1, W2, W3, W4, M1, M2, M3, M4, 2F1, 2F2 and 2F3. In some embodiments, the strains are strains having all of the identifying characteristics of strains W1, W2, W3, W4, M1, M2, M3, M4, 2F1, 2F2 and 2F3. Further, each individual strain (W1, W2, W3, W4, M1, M2, M3, M4, 2F1, 2F2 and 2F3) or any combination of these strains can also provide one or more of the benefits described herein. It will also be clear that addition of other microbial strains, carriers, additives, enzymes, yeast, or the like will also provide one or more benefits or improvement of one or more metrics in an animal and will not constitute a substantially different DFM.

The term “16S rRNA” or “16S ribosomal RNA” means the rRNA constituting the small subunit of prokaryotic ribosomes. In bacteria, this sequence can be used to identify and characterize operational taxonomic units.

The term “sequence identity” or “sequence similarity” as used herein, means that two polynucleotide sequences, a candidate sequence and a reference sequence, are identical (i.e. 100% sequence identity) or similar (i.e. on a nucleotide-by-nucleotide basis) over the length of the candidate sequence. In comparing a candidate sequence to a reference sequence, the candidate sequence may comprise additions or deletions (i.e. gaps) as compared to the reference sequence (which does not comprise additions or deletions) for optimal alignment of the two sequences. Optimal alignment of sequences for determining sequence identity may be conducted using the any number of publicly available local alignment algorithms known in the art such as ALIGN or Megalign (DNASTAR), or by inspection.

The term “percent (%) sequence identity” or “percent (%) sequence similarity,” as used herein with respect to a reference sequence is defined as the percentage of nucleotide residues in a candidate sequence that are identical to the residues in the reference polynucleotide sequence after optimal alignment of the sequences and introducing gaps, if necessary, to achieve the maximum percent sequence identity.

As used herein, “prevent,” “preventing,” “prevention” and grammatical variations thereof refers to a method of partially or completely delaying or precluding the onset or recurrence of a disorder or condition (such as necrotic enteritis) and/or one or more of its attendant symptoms or barring an animal from acquiring or reacquiring a disorder or condition or reducing an animal's risk of acquiring or reacquiring a disorder or condition or one or more of its attendant symptoms.

As used herein, the term “reducing” in relation to a particular trait, characteristic, feature, biological process, or phenomena refers to a decrease in the particular trait, characteristic, feature, biological process, or phenomena. The trait, characteristic, feature, biological process, or phenomena can be decreased by 5%, 10%, 15%, 20%, 25%, 30%, 35%, 40%, 45%, 50%, 55%, 60%, 65%, 70%, 75%, 80%, 85%, 90%, 95%, 100% or greater than 100%.

The term “poultry,” as used herein, means domesticated birds kept by humans for their eggs, their meat or their feathers. These birds are most typically members of the superorder Galloanserae, especially the order Galliformes which includes, without limitation, chickens, quails, ducks, geese, emus, ostriches, pheasant, and turkeys.

As used herein “administer” or “administering” is meant the action of introducing one or more microbial strain, an exogenous feed enzyme and/or a strain and an exogenous feed enzyme to an animal, such as by feeding or by gavage.

As used herein, “effective amount” means a quantity of DFM and/or exogenous enzymes to improve one or more metrics in an animal. Improvement in one or more metrics of an animal (such as, without limitation, any of increased bodyweight gain, intestinal health status, decreased feed conversion ratio (FCR), improved gut barrier integrity, reduced mortality, reduced pathogen infection, and reduced pathogen shedding in feces) can be measured as described herein or by other methods known in the art. An effective amount can be administered to the animal by providing ad libitum access to feed containing the DFM and exogenous enzymes. The DFM and exogenous enzymes can also be administered in one or more doses.

The term “intestinal health status” refers to the status of the gut wall structure and morphology which can be affected by, for example, infectious agents or a non-infectious cause, such as a suboptimal formulated diet. “Gut wall structure and morphology” or “gut barrier integrity” can refer to, without limitation, epithelial damage and epithelial permeability which is characterized by a shortening of villi, a lengthening of crypts and an infiltration of inflammatory cells (such as, without limitation, CD3+ cells). The latter damage and inflammation markers can also be associated with a “severe” macroscopic appearance of the gut-compared to a “normal” appearance-when evaluated using a scoring system such as the one described by Teirlynck et al. (2011).

As used herein, the term “feed” is used synonymously herein with “feedstuff.” Feed broadly refers to a material, liquid or solid, that is used for nourishing an animal, and for sustaining normal or accelerated growth of an animal including newborns or young and developing animals. The term includes a compound, preparation, mixture, or composition suitable for intake by an animal (such as, e.g., for poultry such as quail, ducks, turkeys, and chickens). In some embodiments, a feed or feed composition comprises a basal food composition and one or more feed additives or feed additive compositions. The term “feed additive” as used herein refers to components included for purposes of fortifying basic feed with additional components to promote feed intake, treat or prevent disease, or alter metabolism. Feed additives include pre-mixes.

A “premix,” as referred to herein, may be a composition composed of micro-ingredients such as, but not limited to, one or more of vitamins, minerals, chemical preservatives, antibiotics, fermentation products, and other essential ingredients. Premixes are usually compositions suitable for blending into commercial rations.

As used herein, “improving one or more metrics in an animal” refers to improvments on measurements relevant to the growth and/or health of an animal (such as a domesticated bird, for example, a chicken), measured by one or more of the following parameters: average daily weight gain (ADG), overall weight, mortality, feed conversion (which includes both feed: gain and gain: feed), feed intake, intestinal health status, decreased feed conversion ratio (FCR), improved gut barrier integrity, reduced mortality, reduced pathogen infection, and reduced pathogen shedding in feces. “An improvement in a metric” or “improved metric” as used herein, refers to an improvement in at least one of the parameters listed under the metrics in an animal definition.

As used herein, the term “feed conversion ratio” refers to the amount of feed fed to an animal to increase the weight of the animal by a specified amount. An improved feed conversion ratio means a lower feed conversion ratio. By “lower feed conversion ratio” or “improved feed conversion ratio” it is meant that the use of a feed additive composition in feed results in a lower amount of feed being required to be fed to an animal to increase the weight of the animal by a specified amount compared to the amount of feed required to increase the weight of the animal by the same amount when the feed does not comprise said feed additive composition.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number can be a number which, in the context in which it is presented, provides the substantial equivalent of the specifically recited number. For example, in connection with a numerical value, the term “about” refers to a range of −10% to +10% of the numerical value, unless the term is otherwise specifically defined in context.

As used herein, the singular terms “a,” “an,” and “the” include the plural reference unless the context clearly indicates otherwise.

It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is also noted that the term “consisting essentially of,” as used herein refers to a composition wherein the component(s) after the term is in the presence of other known component(s) in a total amount that is less than 30% by weight of the total composition and do not contribute to or interferes with the actions or activities of the component(s).

It is further noted that the term “comprising,” as used herein, means including, but not limited to, the component(s) after the term “comprising.” The component(s) after the term “comprising” are required or mandatory, but the composition comprising the component(s) can further include other non-mandatory or optional component(s).

It is also noted that the term “consisting of,” as used herein, means including, and limited to, the component(s) after the term “consisting of.” The component(s) after the term “consisting of” are therefore required or mandatory, and no other component(s) are present in the composition.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Unless defined otherwise herein, all technical and scientific terms used herein have the same meaning as commonly understood by one of ordinary skill in the art to which this invention pertains.

Other definitions of terms may appear throughout the specification.

### II. Compositions

### A. Strains

Direct fed microbials (DFMs) refers to the feeding of beneficial microbes to animals, such as domestic birds when they are under periods of stress (disease, ration changes, environmental or production challenges) or as a part of a daily nutritional regimen to prevent disease and facilitate nutrient usage during digestion. Probiotics is another term for this category of feed additives. Probiotics or DFMs have been shown to improve animal performance in controlled studies. In some embodiments, DFMs include both direct fed bacteria and/or yeast-based products and, in particular embodiments, include viable microorganisms. The term “viable microorganism” means a microorganism which is metabolically active or able to differentiate.

In one embodiment, the DFM may be a spore forming bacterium and hence the term DFM may refer to a composition that is comprised of or contain spores, e.g., bacterial spores. Therefore, in one embodiment the term “viable microorganism” as used herein may include microbial spores, such as endospores or conidia. In another embodiment, the DFM in the feed additive composition according to the present invention is not comprised of or does not contain microbial spores, e.g. endospores or conidia (i.e., the DFM is non-spore forming).

The strains provided herein include Anaerotruncus colihominis strain W1, Anaerotruncus colihominis strain W2, Anaerotruncus colihominis strain W3, Anaerotruncus colihominis strain W4, Coprococcus sp. strain M1, Anaerotruncus colihominis strain M2, Clostridium lactatifermentans strain M3, Pseudoflavonifractor capillosus strain M4, Clostridium lactatifermentans strain 2F1, Lactobacillus salivarius strain 2F2, and Lactobacillus reuteri strain 2F3 which are also referred to herein as W1, W2, W3, W4, M1, M2, M3, M4, 2F1, 2F2, and 2F3, respectively.

Anaerotruncus colihominis strain W1, Anaerotruncus colihominis strain W2, Anaerotruncus colihominis strain W3, and Anaerotruncus colihominis strain W4 were deposited on Oct. 9, 2019 at the Westerdijk Fungal Biodiversity Institute (WFDB), Uppsalalaan 8, 3584 CT, Utrecht, The Netherlands and given accession numbers CBS 146120, CBS 146122, CBS 146123, and CBS 146121, respectively. The deposits were made under the provisions of the Budapest Treaty on the International Recognition of the Deposit of Microorganisms for the Purposes of Patent Procedure. One or more strain provided herein can be used as a direct-fed microbial (DFM).

Coprococcus sp. strain M1, Anaerotruncus colihominis strain M2, and Clostridium lactatifermentans strain M3, were deposited on Oct. 9, 2019 at the Westerdijk Fungal Biodiversity Institute (WFDB), Uppsalalaan 8, 3584 CT, Utrecht, The Netherlands and given accession numbers CBS 146125, CBS 146119, and CBS 146124, respectively. The deposits were made under the provisions of the Budapest Treaty on the International Recognition of the Deposit of Microorganisms for the Purposes of Patent Procedure. One or more strain provided herein can be used as a direct-fed microbial (DFM).

Clostridium lactatifermentans strain 2F1, Lactobacillus salivarius strain 2F2, and Lactobacillus reuteri strain 2F3 were deposited on Jul. 24, 2019 and Oct. 9, 2019 at the Westerdijk Fungal Biodiversity Institute (WFDB), Uppsalalaan 8, 3584 CT, Utrecht, The Netherlands and given accession numbers CBS 146124, CBS 146126, and CBS 145921, respectively. The deposits were made under the provisions of the Budapest Treaty on the International Recognition of the Deposit of Microorganisms for the Purposes of Patent Procedure. One or more strain provided herein can be used as a direct-fed microbial (DFM). Multiple strains of direct fed microbials can be combined into a single composition (for example, a feed additive composition or a feed) to form a multi-strain DFM.

DFM compositions can include those that contain one or more strains (such as any of about 1, 2, 3, 4, 5, 6, 7, or 8 or more strains) of Anaerotruncus colihominis. This microbe is a gram-positive, non-spore-forming, rod-shaped and anaerobic bacterium from the genus of Anaerotruncus which has been observed to occur in human feces (Lau et al., J Clin Pathol. 2006 July; 59 (7): 748-752).

The DFM composition can include one or more of A. colihominis strains W1, W2, W3, and/or W4 or one or more microbe(s) having a 16S ribosomal RNA sequence displaying at least about 97.0% sequence similarity (such as any of about 97%, 97.5%, 98%, 98.5%, 99%, 99.5%, or 100% sequence similarity) to a 16S ribosomal RNA sequence of one or more of A. colihominis strains W1 (SEQ ID NO:1), W2 (SEQ ID NO:2), W3 (SEQ ID NO:3), and/or W4 (SEQ ID NO:4). In some embodiments, the DFM composition includes only A. colihominis strain W1, W2, W3, or W4. In another embodiment, DFM composition includes A. colihominis strains W1 and W2; A. colihominis strains W1 and W3; A. colihominis strains W1 and W4; A. colihominis strains W2 and W3; A. colihominis strains W2 and W4; A. colihominis strains W3 and W4; A. colihominis strains W1, W2, and W3; A. colihominis strains W1, W3, and W4; A. colihominis strains W2, W3, and W4; or A. colihominis strains W1, W2, W3, and W4. Additionally, when cultured together, one or more A. colihominis strains W1, W2, W3, and/or W4 have one or more physiological or metabolic properties that individually cultured A. colihominis strains lack. These properties can include, without limitation, changes in the amount and/or type of short chain fatty acid produced, change in metabolic profile, and/or a change in the composition of media in which the bacteria are cultured together (such as butyric acid).

The DFM compositions provided herein can include one or more of A. colihominis strains W1, W2, W3, and/or W4 (i.e. the compositions include the actual bacteria from these strains) and/or one or more culture supernatants derived from the culturing of these strains (individually or in co-culture).

DFM compositions can additionally include those that contain one or more of Coprococcus sp. microbes, Anaerotruncus colihominis microbes, Clostridium lactatifermentans microbes, and/or Pseudoflavonifractor capillosus microbes. Coprococcus is a genus of gram-positive anaerobic cocci which has been observed in the human faecal flora. The genus is bio-chemically closely related to Ruminococcus and phylogenetically to the genus Lachnospira. Clostridium lactatifermentans is a gram positive rod-shaped bacterium with tapered ends showing no motility and no spore formation (van der Wielen et al., Int J Syst Evol Microbiol. 2002 May;52 (Pt 3): 921-5). Pseudoflavonifractor capillosus is a normal gram-negative resident of the healthy human gut. In one comprehensive 16S rDNA sequence-based enumeration of the colonic microbiota of three healthy adult humans it represented, on average, 0.008% of all 16S rDNA sequences and 0.016% of the sequences in its division (Eckburg et. al. Science. 2005 Jun. 10;308 (5728):1635-8)). It is also associated with human cysts and wounds, and been known to cause neonatal sepsis.

The DFM composition can include one or more of Coprococcus sp. strain M1, A. colihominis strain M2, C. lactatifermentans strain M3, and/or a P. capillosus strain (such as P. capillosus strain M4) or one or more microbe(s) having a 16S ribosomal RNA sequence displaying at least about 97.0% sequence similarity (such as any of about 97%, 97.5%, 98%, 98.5%, 99%, 99.5%, or 100% sequence similarity) to a 16S ribosomal RNA sequence of one or more of Coprococcus sp. strain M1 (SEQ ID NO:5), A. colihominis strain M2 (SEQ ID NO:6), C. lactatifermentans strain M3 (SEQ ID NO:7), and/or P. capillosus strain M4 (SEQ ID NO:8). In some embodiments, the DFM composition includes only Coprococcus sp. strain M1, A. colihominis strain M2, C. lactatifermentans strain M3, or a P. capillosus strain (such as P. capillosus strain M4). In another embodiment, the DFM composition includes Coprococcus sp. strain M1 and A. colihominis strain M2; Coprococcus sp. strain M1 and C. lactatifermentans strain M3; Coprococcus sp. strain M1 and a P. capillosus strain (such as P. capillosus strain M4); A. colihominis strains M2 and C. lactatifermentans strain M3; A. colihominis strain M2 and a P. capillosus strain (such as P. capillosus strain M4); C. lactatifermentans strain M3 and a P. capillosus strain (such as P. capillosus strain M4); Coprococcus sp. M1, A. colihominis strain M2, and C. lactatifermentans strain M3; Coprococcus sp. strain M1, C. lactatifermentans strain M3, and a P. capillosus strain (such as P. capillosus strain M4); A. colihominis strain M2, C. lactatifermentans strain M3, and a P. capillosus strain a (such as P. capillosus strain M4); or Coprococcus sp. strain M1, A. colihominis strain M2, C. lactatifermentans strain M3, and a P. capillosus strain (such as P. capillosus strain M4). Additionally, when cultured together, one or more Coprococcus sp. strain M1, A. colihominis strain M2, C. lactatifermentans strain M3,and/or a P. capillosus strain (such as P. capillosus strain M4) have one or more physiological or metabolic properties that individually cultured strains lack. These properties can include, without limitation, changes in the amount and/or type of short chain fatty acid production (such as the production of valeric acid which is only observed when these microbes are cultured together).

The DFM compositions provided herein can include one or more Coprococcus sp. strain M1, A. colihominis strain M2, C. lactatifermentans strain M3, and/or a P. capillosus strain (such as P. capillosus strain M4) (i.e. the compositions include the actual bacteria from these strains) and/or one or more culture supernatants derived from the culturing of these strains (individually or in co-culture).

DFM compositions provided herein can additionally include those that contain one or more of Clostridium lactatifermentans microbes, Lactobacillus salivarius microbes, and/or Lactobacillus reuteri microbes. Clostridium lactatifermentans is a gram positive rod-shaped bacterium with tapered ends showing no motility and no spore formation (van der Wielen et al., Int J Syst Evol Microbiol. 2002 May;52 (Pt 3): 921-5). Lactobacillus salivarius is a lactic acid bacteria which is part of the indigenous microbiota of humans and hamsters (Raftis et al., Appl. Environ. Microbiol. January 2011, 77 (3) 954-965). There has been a recent increase in the number of studies in which the probiotic utility of diverse L. salivarius strains was explored (Neville & O'Toole, Future Microbiol. 2010, 5:759-774). Lactobacillus reuteri is a gram-positive bacterium that naturally inhabits the gut of mammals and birds.

The DFM composition can include one or more of C. lactatifermentans strain 2F1, L. salivarius strain 2F2, and/or L. reuteri strain 2F3 or one or more microbe(s) having a 16S ribosomal RNA sequence displaying at least about 97.0% sequence similarity (such as any of about 97%, 97.5%, 98%, 98.5%, 99%, 99.5%, or 100% sequence similarity) to a 16S ribosomal RNA sequence of one or more of C. lactatifermentans strain 2F1 (SEQ ID NO:9), L. salivarius strain 2F2 (SEQ ID NO:10), and/or L. reuteri strain 2F3 (SEQ ID NO:11). In some embodiments, the DFM composition includes only C. lactatifermentans strain 2F1, L. salivarius strain 2F2, or L. reuteri strain 2F3. In another embodiment, the DFM composition includes C. lactatifermentans strain 2F1 and L. salivarius strain 2F2; C. lactatifermentans strain 2F1 and L. reuteri strain 2F3; L. salivarius strain 2F2 and L. reuteri strain 2F3; C. lactatifermentans strain 2F1, L. salivarius strain 2F2, and L. reuteri strain 2F3. Additionally, when cultured together, one or more C. lactatifermentans strain 2F1, L. salivarius strain 2F2, and/or L. reuteri strain 2F3have one or more physiological or metabolic properties that individually cultured strains lack. These properties can include, without limitation, changes in the amount and/or type of short chain fatty acid produced (such as the production of lactic acid which is only observed when these microbes are cultured together) change in metabolic profile, and/or a change in the composition of media in which the bacteria are cultured together.

The DFM compositions provided herein can include one or more C. lactatifermentans strain 2F1, L. salivarius strain 2F2, and L. reuteri strain 2F3 (i.e. the compositions include the actual bacteria from these strains) and/or one or more culture supernatants derived from the culturing of these strains (individually or in co-culture).

B. Exogenous enzymes

Supplemental enzymes can be used as additives to animal feed, particularly poultry and swine feeds, as a means to improve nutrient utilization and performance characteristics.

In one embodiment, the disclosure relates to a composition comprising one or more DFM (such as DFMs containing any of the microbial strains disclosed herein) and one or more exogenous feed enzymes. In another embodiment, the disclosure relates to a composition comprising, consisting of, or consisting essentially of a multi-strain DFM (such as any of the multi-strain DFM compositions disclosed herein) and one or more exogenous feed enzymes. In one embodiment, the exogenous feed enzymes include, but are not limited to, xylanase, amylase, phytase, beta-glucanase, and protease. In still another embodiment, the composition comprises a feed additive.
1. Xylanases

Xylanase is the name given to a class of enzymes that degrade the linear polysaccharide β-1,4-xylan into xylose, thus breaking down hemicellulose, one of the major components of plant cell walls. Xylanases, e.g., endo-β-xylanases (EC 3.2.1.8) hydrolyze the xylan backbone chain. In one embodiment, provided herein are compositions comprising a multi-strain DFM (such as any of the multi-strain DFM compositions disclosed herein) and one or more xylanase.

In one embodiment, the xylanase may be any commercially available xylanase. Suitably the xylanase may be an endo-1,4-P-d-xylanase (classified as E.G. 3.2.1.8) or a 1,4β-xylosidase (classified as E.G. 3.2.1.37). In one embodiment, the disclosure relates to a DFM in combination with an endoxylanase, e.g. an endo-1,4-P-d-xylanase, and another enzyme. All E. C. enzyme classifications referred to herein relate to the classifications provided in Enzyme Nomenclature—Recommendations (1992) of the nomenclature committee of the International Union of Biochemistry and Molecular Biology—ISBN 0-12-226164-3, which is incorporated herein.

In another embodiment, the xylanase may be a xylanase from Bacillus, Trichoderma, Therinomyces, Aspergillus and Penicillium. In still another embodiment, the xylanase may be the xylanase in Axtra XAP® or Avizyme 1502®, both commercially available products from Danisco A/S. In one embodiment, the xylanase may be a mixture of two or more xylanases. In still another embodiment, the xylanase is an endo-1,4-β-xylanase or a 1,4-β-xylosidase. In yet another embodiment, the xylanase is from an organism selected from the group consisting of: Bacillus, Trichoderma, Thermomyces, Aspergillus, Penicillium, and Humicola. In yet another embodiment, the xylanase may be one or more of the xylanases or one or more of the commercial products recited in Table 1.

TABLE 1
Representative commercial xylanases
Representative examples of commercial xylanases.

| Commercial Name ® | Company | Xylanase type | Xylanase source |
|---|---|---|---|
| Allzyme PT | Alltech | endo-1,4-β-xylanase | Aspergillus Niger |
| Amylofeed | Andrés | endo-1,4-β-xylanase | Aspergillus Niger (phoenicis) |
| | Pintaluba S.A | | |
| Avemix 02 CS | Aveve | endo-1,4-β-xylanase | Trichoderma reesei |
| AveMix XG 10 | Aveve, NL | endo-1,4-β-xylanase | Trichoderma reesei |
| Avizyme 1100 | Danisco | endo-1,4-β-xylanase | Trichoderma longibrachiatum |
| Avizyme 1110 | Danisco | endo-1,4-β-xylanase | Trichoderma longibrachiatum |
| Avizyme 1202 | Danisco | endo-1,4-β-xylanase | Trichoderma longibrachiatum |
| Avizyme 1210 | Danisco | endo-1,4-β-xylanase | Trichoderma longibrachiatum |
| Avizyme 1302 | Danisco | endo-1,4-β-xylanase | Trichoderma longibrachiatum |
| Avizyme 1500 | Danisco | endo-1,4-β-xylanase | Trichoderma longibrachiatum |
| Avizyme 1505 | Danisco | endo-1,4-β-xylanase | Trichoderma longibrachiatum |
| Avizyme SX | Danisco | endo-1,4-β-xylanase | Trichoderma longibrachiatum |
| Belfeed MP 100 | Beldem | endo-1,4-β-xylanase | Bacillus subtilis |
| Biofeed Plus | DSM | endo-1,4-β-xylanase | Humicola insolens |
| Danisco Glycosidase | Danisco Animal | endo-1,4-β-xylanase | Trichoderma reesei |
| (TPT/L) | Nutrition | | |
| Danisco Xylanase | Danisco | endo-1,4-β-xylanase | Trichoderma reesei |
| Econase XT | AB Vista | endo-1,4-β-xylanase | Trichoderma reesei |
| Endofeed ® DC | Andrés | endo-1,4-β-xylanase | Aspergillus Niger |
| | Pintaluba S.A. | | |
| Feedlyve AXL | Lyven | endo-1,4-β-xylanase | Trichoderma longibrachiatum |
| Grindazym GP | Danisco | endo-1,4-β-xylanase | Aspergillus Niger |
| Grindazym GV | Danisco | endo-1,4-β-xylanase | Aspergillus Niger |
| Hostazym X | Huvepharma | endo-1,4-β-xylanase | Trichoderma longibrachiatum |
| Kemzyme Plus Dry | Kemin | endo-1,4-β-xylanase | Trichoderma viride |
| Kemzyme Plus Liquid | Kemin | endo-1,4-β-xylanase | Trichoderma viride |
| Kemzyme W dry | Kemin | endo-1,4-β-xylanase | Trichoderma viride |
| Kemzyme W liquid | Kemin | endo-1,4-β-xylanase | Trichoderma viride |
| Natugrain | BASF | endo-1,4-β-xylanase | Trichoderma longibrachiatum |
| Natugrain TS Plus | BASF | endo-1,4-β-xylanase | Aspergillus Niger |
| Natugrain Wheat | BASF | endo-1,4-β-xylanase | Aspergillus Niger |
| Natugrain ® TS/L | BASF | endo-1,4-β-xylanase | Aspergillus Niger |
| Natuzyme | Bioproton | endo-1,4-β-xylanase | Trichoderma longibrachiatum/ |
| | | | Trichoderma reesei |
| Porzyme 8100 | Danisco | endo-1,4-β-xylanase | Trichoderma longibrachiatum |
| Porzyme 8300 | Danisco | endo-1,4-β-xylanase | Trichoderma longibrachiatum |
| Porzyme 9102 | Danisco | endo-1,4-β-xylanase | Trichoderma longibrachiatum |
| Porzyme 9310/ | Danisco | endo-1,4-β-xylanase | Trichoderma longibrachiatum |
| Avizyme 1310 | | | |
| Porzyme ip 100 | Danisco | endo-1,4-β-xylanase | Trichoderma longibrachiatum |
| Ronozyme AX | DSM | endo-1,4-β-xylanase | Thermomyces lanuginosus gene |
| | | | expressed in Aspergillus oryzae |
| Ronozyme WX | DSM/Novozymes | endo-1,4-β-xylanase | Thermomyces lanuginosus gene |
| | | | expressed in Aspergillus oryzae |
| Rovabio Excel | Adisseo | endo-1,4-β-xylanase | Penicillium funiculosum |
| Roxazyme G2 | DSM/Novozymes | endo-1,4-β-xylanase | Trichoderma longibrachiatum |
| Safizym X | Le Saffre | endo-1,4-β-xylanase | Trichoderma longibrachiatum |
| Xylanase | Lyven | endo-1,4-β-xylanase | Trichoderma longibrachiatum |

In one embodiment, the disclosure relates to a composition comprising a multi-strain DFM (such as any of the multi-strain DFM compositions disclosed herein) and xylanase. In one embodiment, the composition comprises 10-50, 50-100, 100-150, 150-200, 200-250, 250-300, 300-350, 350-400, 400-450, 450-500, 500-550, 550-600, 600-650, 650-700, 700-750, and greater than 750 xylanase units/g of composition.

In one embodiment, the composition comprises 500-1000, 1000-1500, 1500-2000, 2000-2500, 2500-3000, 3000-3500, 3500-4000, 4000-4500, 4500-5000, 5000-5500, 5500-6000, 6000-6500, 6500-7000, 7000-7500, 7500-8000, and greater than 8000 xylanase units/g composition.

It will be understood that one xylanase unit (XU) is the amount of enzyme that releases 0.5 μmol of reducing sugar equivalents (as xylose by the Dinitrosalicylic acid (DNS) assay-reducing sugar method) from an oat-spelt-xylan substrate per min at pH 5.3 and 50° C. (Bailey, et al., Journal of Biotechnology, Volume 23, (3), May 1992, 257-270).
2. Amylases

Amylase is a class of enzymes capable of hydrolysing starch to shorter-chain oligosaccharides, such as maltose. The glucose moiety can then be more easily transferred from maltose to a monoglyceride or glycosylmonoglyceride than from the original starch molecule. The term amylase includes α-amylases (E.G. 3.2.1.1), G4-forming amylases (E.G. 3.2.1.60), β-amylases (E.G. 3.2.1.2) and γ-amylases (E.C. 3.2.1.3). Amylases may be of bacterial or fungal origin, or chemically modified or protein engineered mutants. In one embodiment, provided herein are compositions comprising a multi-strain DFM (such as any of the multi-strain DFM compositions disclosed herein) and one or more amylase.

In one embodiment, the amylase may be a mixture of two or more amylases. In another embodiment, the amylase may be an amylase, e.g. an α-amylase, from Bacillus licheniformis and an amylase, e.g. an α-amylase, from Bacillus amyloliquefaciens. In one embodiment, the α-amylase may be the α-amylase in Axtra XAP® or Avizyme 1502®, both commercially available products from Danisco A/S. In yet another embodiment, the amylase may be a pepsin resistant α-amylase, such as a pepsin resistant Trichoderma (such as Trichoderma reesei) alpha amylase. A suitably pepsin resistant α-amylase is taught in UK application number 101 1513.7 (which is incorporated herein by reference) and PCT/IB2011/053018 (which is incorporated herein by reference).

In one embodiment, the amylase for use in the present invention may be one or more of the amylases in one or more of the commercial products recited in Table 2.

TABLE 2
Representative commercial amylases
Representative examples of commercial amylases.

| Commercial product ® | Company | Amylase type | Amylase source |
|---|---|---|---|
| Amylofeed | Andrés | alpha amylase | Aspergillus oryzae |
| | Pintaluba S.A | | |
| Avizyme 1500 | Danisco | alpha amylase | Bacillus amyloliquefaciens |
| Avizyme 1505 | Danisco | alpha amylase | Bacillus amyloliquefaciens |
| Kemzyme Plus Dry | Kemin | alpha-amylase | Bacillus amyloliquefaciens |
| Kemzyme Plus Liquid | Kemin | alpha-amylase | Bacillus amyloliquefaciens |
| Kemzyme W dry | Kemin | alpha-amylase | Bacillus amyloliquefaciens |
| Kemzyme W liquid | Kemin | alpha-amylase | Bacillus amyloliquefaciens |
| Natuzyme | Bioproton | alpha-amylase | Trichoderma longibrachiatum/ |
| | | | Trichoderma reesei |
| Porzyme 8100 | Danisco | alpha-amylase | Bacillus amyloliquefaciens |
| Porzyme ip100 | Danisco | alpha-amylase | Bacillus amyloliquefaciens |
| Ronozyme A | DSM/Novozymes | alpha-amylase | Bacillus amyloliquefaciens |
| Ronozyme AX | DSM | alpha-amylase | Bacillus amyloliquefaciens |
| Ronozyme ® | DSM/Novozymes | alpha-amylase | Bacillus stearothermophilus |
| RumiStar (L/CT) | | | expressed in Bacillus licheniformis |

it will be understood that one amylase unit (AU) is the amount of enzyme that releases 1 mmol of glucosidic linkages from a water insoluble cross-linked starch polymer substrate per min at pH 6.5 and 37° C. (this may be referred to herein as the assay for determining 1 AU).

In one embodiment, disclosure relates to a composition comprising a multi-strain DFM (such as any of the multi-strain DFM compositions disclosed herein) and amylase. In one embodiment, disclosure relates to a composition comprising a multi-strain DFM, xylanase and amylase. In one embodiment, the composition comprises 10-50, 50-100, 100-150, 150-200, 200-250, 250-300, 300-350, 350-400, 400-450, 450-500, 500-550, 550-600, 600-650, 650-700, 700-750, and greater than 750 amylase units/g composition.

In one embodiment, the composition comprises 500-1000, 1000-1500, 1500-2000, 2000-2500, 2500-3000, 3000-3500, 3500-4000, 4000-4500, 4500-5000, 5000-5500, 5500-6000, 6000-6500, 6500-7000, 7000-7500, 7500-8000, 8000-8500, 8500-9000, 9000-9500, 9500-10000, 10000-11000, 11000-12000, 12000-13000, 13000-14000, 14000-15000 and greater than 15000amylase units/g composition.
3. Proteases

The term protease as used herein is synonymous with peptidase or proteinase. The protease may be a subtilisin (E.G. 3.4.21.62) or a bacillolysin (E.G. 3.4.24.28) or an alkaline serine protease (E.G. 3.4.21.x) or a keratinase (E.G. 3.4.X.X). In one embodiment, the protease is a subtilisin. Suitable proteases include those of animal, vegetable or microbial origin. Chemically modified or protein engineered mutants are also suitable. The protease may be a serine protease or a metalloprotease. e.g., an alkaline microbial protease or a trypsin-like protease. In one embodiment, provided herein are compositions comprising a multi-strain DFM (such as any of the multi-strain DFM compositions disclosed herein) and one or more protease.

Examples of alkaline proteases are subtilisins, especially those derived from Bacillus sp., e.g., subtilisin Novo, subtilisin Carlsberg, subtilisin 309 (see, e.g., U.S. Pat. No. 6,287,841), subtilisin 147, and subtilisin 168 (see, e.g., WO 89/06279). Examples of trypsin-like proteases are trypsin (e.g., of porcine or bovine origin), and Fusarium proteases (see, e.g., WO 89/06270 and WO 94/25583). Examples of useful proteases also include but are not limited to the variants described in WO 92/19729 and WO 98/20115.

In another embodiment, the protease may be one or more of the proteases in one or more of the commercial products recited in Table 3.

TABLE 3
Representative commercial proteases
Representative examples of commercial proteases.

| Commercial product ® | Company | Protease type | Protease source |
|---|---|---|---|
| Avizyme 1100 | Danisco A/S | Subtilisin | Bacillus subtilis |
| Avizyme 1202 | Danisco A/S | Subtilisin | Bacillus subtilis |
| Avizyme 1302 | Danisco A/S | Subtilisin | Bacillus subtilis |
| Avizyme 1500 | Danisco A/S | Subtilisin | Bacillus subtilis |
| Avizyme 1505 | Danisco A/S | Subtilisin | Bacillus subtilis |
| Kemzyme Plus Dry | Kemin | Bacillolysin | Bacillus amyloliquefaciens |
| Kemzyme W dry | Kemin | Bacillolysin | Bacillus amyloliquefaciens |
| Natuzyme | Bioproton | Protease | Trichoderma longibrachiatum/ |
| | | | Trichoderma reesei |
| Porzyme 8300 | Danisco | Subtilisin | Bacillus subtilis |
| Ronozyme ProAct | DSM/Novozymes | Alkaline | Nacardiopsis prasina gene |
| | | serine protease | expressed in Bacillus licheniformis |
| Versazyme/ | Novus | Keratinase | Bacillus licheniformis |
| Cibenza DP100 | | | |

In one embodiment, the protease is selected from the group consisting of subtilisin, a bacillolysin, an alkine serine protease, a keratinase, and a Nocardiopsis protease.

It will be understood that one protease unit (PU) is the amount of enzyme that liberates from the substrate (0.6% casein solution) one microgram of phenolic compound (expressed as tyrosine equivalents) in one minute at pH 7.5 (40 mM Na₂PO₄/lactic acid buffer) and 40° C. This may be referred to as the assay for determining 1 PU.

In one embodiment, disclosure relates to a composition comprising a multi-strain DFM (such as any of the multi-strain DFM compositions disclosed herein) and a protease. In another embodiment, disclosure relates to a composition comprising a multi-strain DFM (such as any of the multi-strain DFM compositions disclosed herein) and a xylanase and a protease. In still another embodiment, the disclosure relates to a composition comprising a multi-strain DFM (such as any of the multi-strain DFM compositions disclosed herein) and an amylase and a protease. In yet another embodiment, the disclosure relates to a composition comprising a multi-strain DFM (such as any of the multi-strain DFM compositions disclosed herein) and a xylanase, an amylase and a protease.

In one embodiment, the composition comprises 10-50, 50-100, 100-150, 150-200, 200-250, 250-300, 300-350, 350-400, 400-450, 450-500, 500-550, 550-600, 600-650, 650-700, 700-750, and greater than 750 protease units/g composition.

In one embodiment, the composition comprises 500-1000, 1000-1500, 1500-2000, 2000-2500, 2500-3000, 3000-3500, 3500-4000, 4000-4500, 4500-5000, 5000-5500, 5500-6000, 6000-6500, 6500-7000, 7000-7500, 7500-8000, 8000-8500, 8500-9000, 9000-9500, 9500-10000, 10000-11000, 11000-12000, 12000-13000, 13000-14000, 14000-15000 and greater than 15000 protease units/g composition.
4. Phytases

In one embodiment, provided herein are compositions comprising a multi-strain DFM (such as any of the multi-strain DFM compositions disclosed herein) and one or more phytase. The phytase for use in the present invention may be classified a 6-phytase (classified as E.C. 3.1.3.26) or a 3-phytase (classified as E.C. 3.1.3.8). In one embodiment, the phytase for use in the present invention may be one or more of the phytases in one or more of the commercial products below in Table 4:

TABLE 4
Representative commercial phytases

| Commercial product ® | Company | Phytase type | Phytase source |
|---|---|---|---|
| Finase | ABVista | 3-phytase | Trichoderma reesei |
| Finase EC | ABVista | 6-phytase | E. coli gene expressed in |
| | | | Trichoderma reesei |
| Natuphos | BASF | 3-phytase | Aspergillus Niger |
| Natuzyme | Bioproton | phytase (type | Trichoderma longibrachiatum/ |
| | | not specified) | Trichoderma reesei |
| OPTIPHOS ® | Huvepharma AD | 6-phytase | E. coli gene expressed in |
| | | | Pichia pastoris |
| Phytase sp1002 | DSM | 3-phytase | A consensus gene expressed in |
| | | | Hansenula polymorpha |
| Phyzyme XP | Danisco | 6-phytase | E. coli gene expressed in |
| | | | Schizosaccahomyces pombe |
| Quantum 2500D, | ABVista | 6-phytase | E. coli gene expressed in |
| 5000L | | | Pichia pastoris or Trichoderma |
| Ronozyme Hi-Phos | DSM/Novozymes | 6-phytase | Citrobacter braakii gene |
| (M/L) | | | expressed in Asperigillus oryzae |
| Ronozyme NP | DSM/Novozymes | 6-phytase | Peniphora lycii gene |
| | | | expressed in Asperigillus oryzae |
| Ronozyme P | DSM/Novozymes | 6-phytase | Peniphora lycii gene |
| | | | expressed in Asperigillus oryzae |
| Rovabio PHY | Adisseo | 3-phytase | Penicillium funiculosum |

In one embodiment the phytase is a Citrobacter phytase derived from e.g. Citrobacter freundii, preferably C. freundii NCIMB 41247 and variants thereof e.g. as disclosed in WO2006/038062 (incorporated herein by reference) and WO2006/038128 (incorporated herein by reference), Citrobacter braakii YH-15 as disclosed in WO 2004/085638, Citrobacter braakii ATCC 51113 as disclosed in WO2006/037328 (incorporated herein by reference), as well as variants thereof e.g. as disclosed in WO2007/112739 (incorporated herein by reference) and WO2011/117396 (incorporated herein by reference), Citrobacter amalonaticus, preferably Citrobacter amalonaticus ATCC 25405 or Citrobacter amalonaticus ATCC 25407 as disclosed in WO2006037327 (incorporated herein by reference), Citrobacter gillenii, preferably Citrobacter gillenii DSM 13694 as disclosed in WO2006037327 (incorporated herein by reference), or Citrobacter intermedius, Citrobacter koseri, Citrobacter murliniae, Citrobacter rodentium, Citrobacter sedlakii, Citrobacter werkmanii, Citrobacter youngae, Citrobacter species polypeptides or variants thereof.

In some embodiments, the phytase is an E. coli phytase marketed under the name Phyzyme XP™ Danisco A/S. Alternatively, the phytase may be a Buttiauxella phytase, e.g. a Buttiauxella agrestis phytase, for example, the phytase enzymes taught in WO 2006/043178, WO 2008/097619, WO2009/129489, WO2008/092901, PCT/US2009/41011 or PCT/IB2010/051804, all of which are incorporated herein by reference.

In one embodiment, the phytase may be a phytase from Hafnia, e.g. from Hafnia alvei, such as the phytase enzyme(s) taught in US2008263688, which reference is incorporated herein by reference. In one embodiment, the phytase may be a phytase from Aspergillus, e.g. from Apergillus orzyae. In one embodiment, the phytase may be a phytase from Penicillium, e.g. from Penicillium funiculosum.

Preferably, the phytase is present in the feedstuff in range of about 200 FTU/kg to about 1000 FTU/kg feed, more preferably about 300 FTU/kg feed to about 750 FTU/kg feed, more preferably about 400 FTU/kg feed to about 500 FTU/kg feed. In one embodiment, the phytase is present in the feedstuff at more than about 200 FTU/kg feed, suitably more than about 300 FTU/kg feed, suitably more than about 400 FTU/kg feed. In one embodiment, the phytase is present in the feedstuff at less than about 1000 FTU/kg feed, suitably less than about 750 FTU/kg feed. Preferably, the phytase is present in the feed additive composition in range of about 40 FTU/g to about 40,000 FTU/g composition, more preferably about 80 FTU/g composition to about 20,000 FTU/g composition, and even more preferably about 100 FTU/g composition to about 10,000 FTU/g composition, and even more preferably about 200 FTU/g composition to about 10,000 FTU/g composition. In one embodiment, the phytase is present in the feed additive composition at more than about 40 FTU/g composition, suitably more than about 60 FTU/g composition, suitably more than about 100 FTU/g composition, suitably more than about 150 FTU/g composition, suitably more than about 200 FTU/g composition. In one embodiment, the phytase is present in the feed additive composition at less than about 40,000 FTU/g composition, suitably less than about 20,000 FTU/g composition, suitably less than about 15,000 FTU/g composition, suitably less than about 10,000 FTU/g composition.

It will be understood that as used herein 1 FTU (phytase unit) is defined as the amount of enzyme required to release 1 umol of inorganic orthophosphate from a substrate in one minute under the reaction conditions defined in the ISO 2009 phytase assay—A standard assay for determining phytase activity and 1 FTU can be found at International Standard ISO/DIS 30024:1-17, 2009. In one embodiment, the enzyme is classified using the E. C. classification above, and the E.C. classification designates an enzyme having that activity when tested in the assay taught herein for determining 1 FTU.

### C. DFM Formulations

In one embodiment, the DFM (such as any of the multi-strain DFM compositions disclosed herein) and, optionally, exogenous enzymes may be formulated as a liquid, a dry powder or a granule. In one embodiment, the DFMs and exogenous enzymes can be formulated as a single mixture. In another embodiment, the DFMs and the exogenous enzymes can be formulated as separate mixtures. In still another embodiment, separate mixtures of DFMs and the exogenous enzymes can be administered at the same time or at different times. In still another embodiment, separate mixtures of DFMs and the exogenous enzymes can be administered simultaneously or sequentially. In yet another embodiment, a first mixture comprising DFMs can be administered followed by a second mixture comprising exogenous enzymes. In still another embodiment, a first mixture comprising exogenous enzymes can be administered followed by a second mixture comprising DFMs.

The dry powder or granules may be prepared by means known to those skilled in the art, such as, in top-spray fluid bed coater, in a buttom spray Wurster or by drum granulation (e.g. High sheer granulation), extrusion, pan coating or in a microingredients mixer.

In another embodiment, the DFM and/or the enzyme(s) may be coated, for example encapsulated. Suitably the DFM and enzymes may be formulated within the same coating or encapsulated within the same capsule. Alternatively, one or more of the enzymes may be formulated within the same coating or encapsulated within the same capsule while the DFM can be formulated in a separate coating from the enzymes.

In some embodiments, such as where the DFM is capable of producing endospores, the DFM may be provided without any coating. In such circumstances, the DFM endospores may be simply admixed with one or more enzymes. In the latter case, the enzymes may be coated, e.g. encapsulated, for instance one or more or all of the enzymes may be coated, e.g. encapsulated. The enzymes may be encapsulated as mixtures (i.e. comprising one or more, two or more, three or more or all) of enzymes or they may be encapsulated separately, e.g. as single enzymes. In one preferred embodiment, all enzymes may be coated, e.g. encapsulated, together. In one embodiment, the coating protects the enzymes from heat and may be considered a thermoprotectant.

In another embodiment, the DFMs and exogenous feed enzymes may be mixed with feed or administered in the drinking water. In one embodiment, the dosage range for inclusion into water is about 1×10³ CFU/animal/day to about 1×10¹⁰ CFU/animal/day, for example, about 1×10⁷ CFU/animal/day.

### D. Feed Additive Compositions

In one embodiment, provided herein are feed additive compositions comprising one or more DFMs (such as any of the multi-strain DFMs disclosed herein) and, optionally, one or more exogenous feed enzymes. In one embodiment, the feed additive composition can be formulated in any suitable way to ensure that the formulation comprises viable DFMs and, optionally, active enzymes.

In one embodiment, the feed additive composition may be used in the form of solid or liquid preparations or alternatives thereof. Examples of solid preparations include powders, pastes, boluses, capsules, ovules, pills, pellets, tablets, dusts, and granules which may be wettable, spray-dried or freeze-dried. Examples of liquid preparations include, but are not limited to, aqueous, organic or aqueous-organic solutions, suspensions and emulsions.

In another embodiment, the feed additive composition can be used in a solid form. In one embodiment, the solid form is a pelleted form. In solid form, the feed additive composition may also contain one or more of: excipients such as microcrystalline cellulose, lactose, sodium citrate, calcium carbonate, dibasic calcium phosphate and glycine; disintegrants such as starch (preferably corn, potato or tapioca starch), sodium starch glycollate, croscarmellose sodium and certain complex silicates; granulation binders such as polyvinylpyrrolidone, hydroxypropylmethylcellulose (HPMC), hydroxypropylcellulose (HPC), sucrose, gelatin and acacia; lubricating agents such as magnesium stearate, stearic acid, glyceryl behenate and talc may be included.

Examples of nutritionally acceptable carriers for use in preparing the forms include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

In one embodiment, the feed additive composition is formulated to a dry powder or granules as described in WO2007/044968 (referred to as TPT granules) or WO 1997/016076 or WO 1992/012645 (each of which is incorporated herein by reference).

In one embodiment, the feed additive composition may be formulated to a granule feed composition comprising: an active agent comprising one or more DFM (such as any of the multi-strain DFM compositions disclosed herein) and, optionally, one or more exogenous feed enzyme and at least one coating. In one embodiment, the active agent of the granule retains activity after processing. In one embodiment, the active agent of the granule retains an activity level after processing selected from the group consisting of: 50-60% activity, 60-70% activity, 70-80% activity, 80-85% activity, 85-90% activity, and 90-95% activity.

In another embodiment, the granule may contain one coating. The coating may comprise a moisture hydrating material that constitutes at least 55% w/w of the granule. In another embodiment, the granule may contain two coatings. The two coatings may be a moisture hydrating coating and a moisture barrier coating. In some embodiments, the moisture hydrating coating may be from 25% to 60% w/w of the granule and the moisture barrier coating may be from 2% to 15% w/w of the granule. The moisture hydrating coating may be selected from inorganic salts, sucrose, starch, and maltodextrin and the moisture barrier coating may be selected from polymers, gums, whey and starch.

In yet another embodiment, the granule may be produced using a feed pelleting process and the feed pretreatment process may be conducted between 70° C. and 95° C. for up to several minutes, such as between 85° C. and 95° C. In another embodiment, the granule may be produced using a steam-heated pelleting process that may be conducted between 85° C. and 95° C. for up to several minutes.

In one embodiment, the granule may have a moisture barrier coating selected from polymers and gums and the moisture hydrating material may be an inorganic salt. The moisture hydrating coating may be between 25% and 45% w/w of the granule and the moisture barrier coating may be between 2% and 20% w/w of the granule.

In one embodiment, the active agent retains activity after conditions selected from one or more of: (a) a feed pelleting process; (b) a steam-heated feed pretreatment process; (c) storage; (d) storage as an ingredient in an unpelleted mixture; and (e) storage as an ingredient in a feed base mix or a feed premix comprising at least one compound selected from trace minerals, organic acids, reducing sugars, vitamins, choline chloride, and compounds which result in an acidic or a basic feed base mix or feed premix.

In some embodiments, the DFM (e.g. DFM endospores, for example) may be diluted using a diluent, such as starch powder, lime stone or the like. In one embodiment, the DFM and the enzymes may be in a liquid formulation suitable for consumption preferably such liquid consumption contains one or more of the following: a buffer, salt, sorbitol and/or glycerol. In another embodiment, the feed additive composition may be formulated by applying, e.g. spraying, the enzyme(s) onto a carrier substrate, such as ground wheat for example.

In one embodiment, the feed additive composition may be formulated as a premix. By way of example only, the premix may comprise one or more feed components, such as one or more minerals and/or one or more vitamins.

In one embodiment, the DFM and exogenous feed enzymes may be formulated with at least one physiologically acceptable carrier selected from at least one of maltodextrin, limestone (calcium carbonate), cyclodextrin, wheat or a wheat component, sucrose, starch, Na2SO4, Talc, PVA, sorbitol, benzoate, sorbiate, glycerol, sucrose, propylene glycol, 1,3-propane diol, glucose, parabens, sodium chloride, citrate, acetate, phosphate, calcium, metabisulfite, formate and mixtures thereof.

In another embodiment, the feed additive composition can be delivered as an aqueous suspension and/or an elixir. The feed additive composition may be combined with various sweetening or flavoring agents, coloring matter or dyes, with emulsifying and/or suspending agents and with diluents such as water, propylene glycol and glycerin, and combinations thereof.

### E. Feedstuffs

In another embodiment, provided herein are feed additive compositions containing any of the multi-strain DFM compositions disclosed herein that may be used as a feed or in the preparation of a feed. The feed may be in the form of a solution or as a solid depending on the use and/or the mode of application and/or the mode of administration. When used as a feed or in the preparation of a feed, such as functional feed, the feed additive composition may be used in conjunction with one or more of the following: a nutritionally acceptable carrier, a nutritionally acceptable diluent, a nutritionally acceptable excipient, a nutritionally acceptable adjuvant, a nutritionally active ingredient.

In one embodiment, the feed additive composition disclosed herein is admixed with a feed component to form a feedstuff. In one embodiment, the feed may be a fodder, or a premix thereof, a compound feed, or a premix thereof. In one embodiment, the feed additive composition disclosed herein may be admixed with a compound feed, a compound feed component or a premix of a compound feed or to a fodder, a fodder component, or a premix of a fodder.

In one embodiment, fodder may be obtained from one or more of the plants selected from: alfalfa (lucerne), barley, birdsfoot trefoil, brassicas, Chau moellier, kale, rapeseed (canola), rutabaga (swede), turnip, clover, alsike clover, red clover, subterranean clover, white clover, grass, false oat grass, fescue, Bermuda grass, brome, heath grass, meadow grasses (from naturally mixed grassland swards, orchard grass, rye grass, Timothy-grass, corn (maize), millet, oats, sorghum, soybeans, trees (pollard tree shoots for tree-hay), wheat, and legumes.

Compound feeds can be complete feeds that provide all the daily required nutrients, concentrates that provide a part of the ration (protein, energy) or supplements that only provide additional micronutrients, such as minerals and vitamins. The main ingredients used in compound feed are the feed grains, which include com, soybeans, sorghum, oats, and barley.

A “premix,” as referred to herein, may be a composition composed of micro-ingredients such as vitamins, minerals, chemical preservatives, antibiotics, fermentation products, and other essential ingredients. Premixes are usually compositions suitable for blending into commercial rations.

In one embodiment, a feedstuff as disclosed herein may comprise one or more feed materials selected from the group comprising cereals, such as small grains (e.g., wheat, barley, rye, oats and combinations thereof) and/or large grains such as maize or sorghum; by products from cereals, such as com gluten meal, Distillers Dried Grain Solubles (DDGS), wheat bran, wheat middlings, wheat shorts, rice bran, rice hulls, oat hulls, palm kernel, and citrus pulp; protein obtained from sources such as soya, sunflower, peanut, lupin, peas, fava beans, cotton, canola, fish meal, dried plasma protein, meat and bone meal, potato protein, whey, copra, sesame; oils and fats obtained from vegetable and animal sources; and minerals and vitamins.

In yet another embodiment, a feedstuff may comprise at least one high fiber feed material and/or at least one by-product of the at least one high fiber feed material to provide a high fiber feedstuff. Examples of high fiber feed materials include: wheat, barley, rye, oats, by products from cereals, such as com gluten meal, Distillers Dried Grain Solubles (DDGS), wheat bran, wheat middlings, wheat shorts, rice bran, rice hulls, oat hulls, palm kernel, and citrus pulp. Some protein sources may also be regarded as high fiber: protein obtained from sources such as sunflower, lupin, fava beans and cotton

In still another embodiment, the feed may be one or more of the following: a compound feed and premix, including pellets, nuts or (cattle) cake; a crop or crop residue: com, soybeans, sorghum, oats, barley, com stover, copra, straw, chaff, sugar beet waste; fish meal; freshly cut grass and other forage plants; meat and bone meal; molasses; oil cake and press cake; oligosaccharides; conserved forage plants: hay and silage; seaweed; seeds and grains, either whole or prepared by crushing, milling etc.; sprouted grains and legumes; yeast extract.

In one embodiment, the feed additive composition of disclosed herein is admixed with the product (e.g. feedstuff). Alternatively, the feed additive composition may be included in the emulsion or raw ingredients of a feedstuff. In another embodiment, the feed additive composition is made available on or to the surface of a product to be affected/treated. In still another embodiment, the feed additive compositions disclosed herein may be applied, interspersed, coated and/or impregnated to a product (e.g. feedstuff or raw ingredients of a feedstuff) with a controlled amount of DFM and, optionally, enzymes.

In yet another embodiment, the DFM and optional enzymes may be used simultaneously (e.g. when they are in admixture together or even when they are delivered by different routes) or sequentially (e.g. they may be delivered by different routes).

In one embodiment, the DFM and optional enzymes are applied to the feedstuff simultaneously. In yet another embodiment, the DFM and optional enzymes are admixed prior to being delivered to a feedstuff or to a raw ingredient of a feedstuff.

In one embodiment, the DFMs in the feed additive compositions disclosed herein can be added in suitable concentrations including but not limited to concentrations in the final feed product that offer a daily dose of from about 2×10³ CFU to about 2×10¹¹ CFU, from about 2×10⁶ to about 1×10¹⁰, and from about 3.75×10⁷ CFU to about 1×10¹⁰ CFU.

### III. Methods

### A. Methods for Improving Performance Metrics in an Animal

Further provided herein are methods for increasing performance metrics of an animal. In another embodiment, the disclosure relates to methods of increasing performance metrics of a bird. In still another embodiment, the disclosure relates to methods of increasing performance metrics of poultry, including but not limited to broilers, chickens and turkeys.

In yet another embodiment, the disclosure relates to a method comprising administering to an animal a composition comprising DFMs (such as any of the multi-strain DFMs disclosed herein) and, optionally, exogenous feed enzymes. In still another embodiment, the disclosure relates to a method comprising administering to an animal an effective amount of a composition comprising DFMs and optional exogenous feed enzymes to increase performance of the animal. This effective amount can be administered to the animal in one or more doses. In one embodiment, the animal is poultry. In still another embodiment, the animal is a broiler.

In another embodiment, the disclosure relates to a method comprising administering to an animal (such as a domesticated bird, for example, a chicken) an effective amount of a composition comprising DFMs (such as any of the multi-strain DFMs disclosed herein) and optionally exogenous feed enzymes to increase average daily feed intake. In some embodiments, the average daily feed intake increases by any of about 10%, 15%, 20%, 25%, 30%, 35%, 40%, 45%, 50%, 55%, 60%, 65%, 70%, 75%, 80%, 85%, 90%, 95%, 100%, 105%, or 110%, inclusive of all values falling in between these percentages, relative to animals who are not administered one or more of the multi-strain DFM compositions disclosed herein. In some embodiments, the composition is a feed additive composition. In other embodiments, the composition is a feed or feedstuff.

In another embodiment, the disclosure relates to a method comprising administering to an animal (such as a domesticated bird, for example, a chicken) an effective amount of a composition comprising DFMs (such as any of the multi-strain DFMs disclosed herein) and optional exogenous feed enzymes to increase average daily weight gain. In some embodiments, the average daily weight gain increases by any of about 10%, 15%, 20%, 25%, 30%, 35%, 40%, 45%, 50%, 55%, 60%, 65%, 70%, 75%, 80%, 85%, 90%, 95%, 100%, 105%, or 110%, inclusive of all values falling in between these percentages, relative to animals who are not administered one or more of the multi-strain DFM compositions disclosed herein. In some embodiments, the composition is a feed additive composition. In other embodiments, the composition is a feed or feedstuff.

In another embodiment, the disclosure relates to a method comprising administering to an animal (such as a domesticated bird, for example, a chicken) an effective amount of a composition comprising DFMs (such as any of the multi-strain DFMs disclosed herein) and optional exogenous feed enzymes to increase total weight gain. In some embodiments, total weight gain increases by any of about 10%, 15%, 20%, 25%, 30%, 35%, 40%, 45%, 50%, 55%, 60%, 65%, 70%, 75%, 80%, 85%, 90%, 95%, 100%, 105%, or 110%, inclusive of all values falling in between these percentages, relative to animals who are not administered one or more of the multi-strain DFM compositions disclosed herein. In some embodiments, the composition is a feed additive composition. In other embodiments, the composition is a feed or feedstuff.

In another embodiment, the disclosure relates to a method comprising administering to an animal (such as a domesticated bird, for example, a chicken) an effective amount of a composition comprising DFMs (such as any of the multi-strain DFMs disclosed herein) and optional exogenous feed enzymes to increase feed conversion, which can be measured by either feed: gain or gain: feed. In some embodiments, feed conversion increases by any of about 10%, 15%, 20%, 25%, 30%, 35%, 40%, 45%, 50%, 55%, 60%, 65%, 70%, 75%, 80%, 85%, 90%, 95%, 100%, 105%, or 110%, inclusive of all values falling in between these percentages, relative to animals who are not administered one or more of the multi-strain DFM compositions disclosed herein. In some embodiments, the composition is a feed additive composition. In other embodiments, the composition is a feed or feedstuff.

In another embodiment, the disclosure relates to a method comprising administering to an animal (such as a domesticated bird, for example, a chicken) an effective amount of a composition comprising DFMs (such as any of the multi-strain DFMs disclosed herein) and optional exogenous feed enzymes to increase feed efficiency. In some embodiments, feed efficiency increases by any of about 10%, 15%, 20%, 25%, 30%, 35%, 40%, 45%, 50%, 55%, 60%, 65%, 70%, 75%, 80%, 85%, 90%, 95%, 100%, 105%, or 110%, inclusive of all values falling in between these percentages, relative to animals who are not administered one or more of the multi-strain DFM compositions disclosed herein. In some embodiments, the composition is a feed additive composition. In other embodiments, the composition is a feed or feedstuff.

In another embodiment, the disclosure relates to a method comprising administering to an animal (such as a domesticated bird, for example, a chicken) an effective amount of a composition comprising DFMs (such as any of the multi-strain DFMs disclosed herein) and optional exogenous feed enzymes to decrease mortality. In some embodiments, mortality decreases by any of about 10%, 15%, 20%, 25%, 30%, 35%, 40%, 45%, 50%, 55%, 60%, 65%, 70%, 75%, 80%, 85%, 90%, 95%, or 100%, inclusive of all values falling in between these percentages, relative to animals who are not administered one or more of the multi-strain DFM compositions disclosed herein. In some embodiments, the composition is a feed additive composition. In other embodiments, the composition is a feed or feedstuff.

In another embodiment, the disclosure relates to a method comprising administering to an animal (such as a domesticated bird, for example, a chicken) an effective amount of a composition comprising DFMs (such as any of the multi-strain DFMs disclosed herein) and optional exogenous feed enzymes to decrease feed conversion ratio (FCR). In some embodiments, FCR decreases by any of about 10%, 15%, 20%, 25%, 30%, 35%, 40%, 45%, 50%, 55%, 60%, 65%, 70%, 75%, 80%, 85%, 90%, 95%, or 100%, inclusive of all values falling in between these percentages, relative to animals who are not administered one or more of the multi-strain DFM compositions disclosed herein. In some embodiments, the composition is a feed additive composition. In other embodiments, the composition is a feed or feedstuff.

In another embodiment, the disclosure relates to a method comprising administering to an animal (such as a domesticated bird, for example, a chicken) an effective amount of a composition comprising DFMs (such as any of the multi-strain DFMs disclosed herein) and optional exogenous feed enzymes to increase gut barrier integrity. In some embodiments, gut barrier integrity increases by any of about 10%, 15%, 20%, 25%, 30%, 35%, 40%, 45%, 50%, 55%, 60%, 65%, 70%, 75%, 80%, 85%, 90%, 95%, 100%, 105%, or 110%, inclusive of all values falling in between these percentages, relative to animals who are not administered one or more of the multi-strain DFM compositions disclosed herein. “Gut barrier integrity” can refer to, without limitation, epithelial damage and epithelial permeability which is characterized by a shortening of villi, a lengthening of crypts and an infiltration of inflammatory cells (such as, without limitation, CD3+ cells). The latter damage and inflammation markers can also be associated with a “severe” macroscopic appearance of the gut—compared to a “normal” appearance-when evaluated using a scoring system such as the one described by Teirlynck et al. (2011). In some embodiments, the composition is a feed additive composition. In other embodiments, the composition is a feed or feedstuff.

In another embodiment, the disclosure relates to a method comprising administering to an animal (such as a domesticated bird, for example, a chicken) an effective amount of a composition comprising DFMs (such as any of the multi-strain DFMs disclosed herein) and optional exogenous feed enzymes to decrease or prevent pathogen infection (such as, without limitation, infection by Clostridium perfringens, Campylobacter jejuni, a Salmonela sp., and/or Escherichia coli). In some embodiments, pathogen infection decreases by any of about 10%, 15%, 20%, 25%, 30%, 35%, 40%, 45%, 50%, 55%, 60%, 65%, 70%, 75%, 80%, 85%, 90%, 95%, or 100%, inclusive of all values falling in between these percentages, relative to animals who are not administered one or more of the multi-strain DFM compositions disclosed herein. In some embodiments, the composition is a feed additive composition. In other embodiments, the composition is a feed or feedstuff. In some embodiments, the composition is a feed additive composition. In other embodiments, the composition is a feed or feedstuff.

In another embodiment, the disclosure relates to a method comprising administering to an animal (such as a domesticated bird, for example, a chicken) an effective amount of a composition comprising DFMs (such as any of the multi-strain DFMs disclosed herein) and optional exogenous feed enzymes to decrease or prevent pathogen shedding in the feces (such as, without limitation, shedding of Clostridium perfringens, Campylobacter jejuni, a Salmonela sp., and/or Escherichia coli). In some embodiments, pathogen shedding in the feces decreases by any of about 10%, 15%, 20%, 25%, 30%, 35%, 40%, 45%, 50%, 55%, 60%, 65%, 70%, 75%, 80%, 85%, 90%, 95%, or 100%, inclusive of all values falling in between these percentages, relative to animals who are not administered one or more of the multi-strain DFM compositions disclosed herein. In some embodiments, the composition is a feed additive composition. In other embodiments, the composition is a feed or feedstuff.

In still another embodiment, the DFM composition (such as a feed or feed additive composition) administered to the animal (such as a domesticated bird, for example, a chicken) is a multi-strain DFM comprising one or more of A. colihominis strain W1 (CBS 146120), or a strain having all of the identifying characteristics of A. colihominis strain W1, or a microbe having a 16S ribosomal RNA sequence displaying at least about 97.0% sequence similarity (such as any of about 97%, 97.5%, 98%, 98.5%, 99%, 99.5%, or 100% sequence similarity) to a 16S ribosomal RNA sequence of A. colihominis strain W1 (SEQ ID NO:1); Anaerotruncus colihominis strain W2 (CBS 146122), or a strain having all of the identifying characteristics of A. colihominis strain W2, or a microbe having a 16S ribosomal RNA sequence displaying at least about 97.0% sequence similarity (such as any of about 97%, 97.5%, 98%, 98.5%, 99%, 99.5%, or 100% sequence similarity) to a 16S ribosomal RNA sequence of A. colihominis strain W2 (SEQ ID NO:2); Anaerotruncus colihominis strain W3 (CBS 146123), or a strain having all of the identifying characteristics of A. colihominis strain W3, or a microbe having a 16S ribosomal RNA sequence displaying at least about 97.0% sequence similarity (such as any of about 97%, 97.5%, 98%, 98.5%, 99%, 99.5%, or 100% sequence similarity) to a 16S ribosomal RNA sequence of A. colihominis strain W3 (SEQ ID NO:3); and/or Anaerotruncus colihominis strain W4 (CBS 146121), or a strain having all of the identifying characteristics of A. colihominis strain W4, or a microbe having a 16S ribosomal RNA sequence displaying at least about 97.0% sequence similarity (such as any of about 97%, 97.5%, 98%, 98.5%, 99%, 99.5%, or 100% sequence similarity) to a 16S ribosomal RNA sequence of A. colihominis strain W4 (SEQ ID NO: 4). In another embodiment, the DFM composition includes A. colihominis strains W1 and W2; A. colihominis strains W1 and W3; A. colihominis strains W1 and W4; A. colihominis strains W2 and W3; A. colihominis strains W2 and W4; A. colihominis strains W3 and W4; A. colihominis strains W1, W2, and W3; A. colihominis strains W1, W3, and W4; A. colihominis strains W2, W3, and W4; or A. colihominis strains W1, W2, W3, and W4. In some embodiments, the one or more A. colihominis strain(s) is (are) administered to an animal at a rate of at least 1×10⁴ CFU/animal/day. For poultry, according to one non-limiting embodiment, the one or more A. colihominis strain(s) can be fed at about 1×10⁵ CFU/g feed to about 1×10¹⁰ CFU/g feed. In at least some embodiments, the one or more A. colihominis strains is (are) fed at about 1×10⁵ CFU/bird/day or about 1×10⁸ CFU/bird/day.

In still another embodiment, the DFM composition (such as a feed or feed additive composition) administered to the animal (such as a domesticated bird, for example, a chicken) is a multi-strain DFM comprising one or more of Coprococcus sp. strain M1 (CBS 146125), or a strain having all of the identifying characteristics Coprococcus sp. strain M1, or a microbe having a 16S ribosomal RNA sequence displaying at least about 97.0% sequence similarity (such as any of about 97%, 97.5%, 98%, 98.5%, 99%, 99.5%, or 100% sequence similarity) to a 16S ribosomal RNA sequence of Coprococcus sp. strain M1 (SEQ ID NO:5); A. colihominis strain M2 (CBS 146119), or a strain having all of the identifying characteristics of A. colihominis strain M2, or a microbe having a 16S ribosomal RNA sequence displaying at least about 97.0% sequence similarity (such as any of about 97%, 97.5%, 98%, 98.5%, 99%, 99.5%, or 100% sequence similarity) to a 16S ribosomal RNA sequence of A. colihominis strain M2 (SEQ ID NO: 6); C. lactatifermentans strain M3 (CBS 146124), or a strain having all of the identifying characteristics of C. lactatifermentans strain M3, or a microbe having a 16S ribosomal RNA sequence displaying at least about 97.0% sequence similarity (such as any of about 97%, 97.5%, 98%, 98.5%, 99%, 99.5%, or 100% sequence similarity) to a 16S ribosomal RNA sequence of C. lactatifermentans strain M3 (SEQ ID NO:7); and/or a P. capillosus strain (such as P. capillosus strain M4) or a microbe having a 16S ribosomal RNA sequence displaying at least about 97.0% sequence similarity (such as any of about 97%, 97.5%, 98%, 98.5%, 99%, 99.5%, or 100% sequence similarity) to a 16S ribosomal RNA sequence of P. capillosus strain M4 (SEQ ID NO: 8). In some embodiments, the DFM composition includes only Coprococcus sp. strain M1, A. colihominis strain M2, C. lactatifermentans strain M3, or a P. capillosus strain (such as P. capillosus strain M4). In another embodiment, the DFM composition includes Coprococcus sp. strain M1 and A. colihominis strain M2; Coprococcus sp. strain M1 and C. lactatifermentans strain M3; Coprococcus sp. strain M1 and a P. capillosus strain (such as P. capillosus strain M4); A. colihominis strains M2 and C. lactatifermentans strain M3; A. colihominis strain M2 and a P. capillosus strain (such as P. capillosus strain M4); C. lactatifermentans strain M3 and a P. capillosus strain (such as P. capillosus strain M4); Coprococcus sp. M1, A. colihominis strain M2, and C. lactatifermentans strain M3; Coprococcus sp. strain M1, C. lactatifermentans strain M3, and a P. capillosus strain (such as P. capillosus strain M4); A. colihominis strain M2, C. lactatifermentans strain M3, and P. capillosus strain M4; or Coprococcus sp. strain M1, A. colihominis strain M2, C. lactatifermentans strain M3, and a P. capillosus strain (such as P. capillosus strain M4). In some embodiments, the one or more M1, M2, M3, and/or M4 strain(s) is (are) administered to an animal at a rate of at least 1×10⁴ CFU/animal/day. For poultry, according to one non-limiting embodiment, the one or more M1, M2, M3, and/or M4 strain(s) can be fed at about 1×10⁵ CFU/g feed to about 1×10¹⁰ CFU/g feed. In at least some embodiments, the one or more M1, M2, M3, and/or M4 strains is (are) fed at about 1×10⁵ CFU/bird/day or about 1×10⁸ CFU/bird/day.

In still another embodiment, the DFM composition (such as a feed or feed additive composition) administered to the animal (such as a domesticated bird, for example, a chicken) is a multi-strain DFM comprising one or more of C. lactatifermentans strain 2F1 (CBS 146124), or a strain having all of the identifying characteristics C. lactatifermentans strain 2F1, or a microbe having a 16S ribosomal RNA sequence displaying at least about 97.0% sequence similarity (such as any of about 97%, 97.5%, 98%, 98.5%, 99%, 99.5%, or 100% sequence similarity) to a 16S ribosomal RNA sequence of C. lactatifermentans strain 2F1 (SEQ ID NO:9); L. salivarius strain 2F2 (CBS 146126), or a strain having all of the identifying characteristics of L. salivarius strain 2F2, or a microbe having a 16S ribosomal RNA sequence displaying at least about 97.0% sequence similarity (such as any of about 97%, 97.5%, 98%, 98.5%, 99%, 99.5%, or 100% sequence similarity) to a 16S ribosomal RNA sequence of L. salivarius strain 2F2 (SEQ ID NO: 10); and/or L. reuteri strain 2F3 (CBS 145921), or a strain having all of the identifying characteristics of L. reuteri strain 2F3, or a microbe having a 16S ribosomal RNA sequence displaying at least about 97.0% sequence similarity (such as any of about 97%, 97.5%, 98%, 98.5%, 99%, 99.5%, or 100% sequence similarity) to a 16S ribosomal RNA sequence of L. reuteri strain 2F3 (SEQ ID NO:11). In some embodiments, L. reuteri strain 2F3 produces reuterin (3-hydroxypropionaldehyde). In other embodiments, L. reuteri strain 2F3 does not produce reuterin (3-hydroxypropionaldehyde). In some embodiments, the DFM composition includes only C. lactatifermentans strain 2F1, L. salivarius strain 2F2, or L. reuteri strain 2F3. In another embodiment, the DFM composition includes C. lactatifermentans strain 2F1 and L. salivarius strain 2F2; C. lactatifermentans strain 2F1 and L. reuteri strain 2F3; L. salivarius strain 2F2 and L. reuteri strain 2F3; or C. lactatifermentans strain 2F1, L. salivarius strain 2F2, and L. reuteri strain 2F3. In some embodiments, the one or more 2F1, 2F2 and/or 2F3 strain(s) is (are) administered to an animal at a rate of at least 1×104 CFU/animal/day. For poultry, according to one non-limiting embodiment, the one or more 2F1, 2F2 and/or 2F3 strain(s) can be fed at about 1×10⁵ CFU/g feed to about 1×10¹⁰ CFU/g feed. In at least some embodiments, the one or more 2F1, 2F2 and/or 2F3 strains is (are) fed at about 1×10⁵ CFU/bird/day or about 1×10⁸ CFU/bird/day.

The DFM compositions provided herein can be administered, for example, as a strain-containing culture solution, a strain-containing supernatant, or a bacterial product of a culture solution. Administration of a composition comprising a DFM and optional exogenous feed enzymes provided herein to an animal can increase the performance of the animal. In one embodiment, administration of a DFM provided herein to an animal can increase the average daily feed intake (ADFI), average daily gain (ADG), or feed efficiency (gain: feed; G:F) (collectively, “performance metrics”). One or more than one of these performance metrics may be improved.

The composition comprising DFMs and exogenous feed enzymes may be administered to the animal in one of many ways. For example, the composition can be administered in a solid form as a veterinary pharmaceutical, may be distributed in an excipient, preferably water, and directly fed to the animal, may be physically mixed with feed material in a dry form, or the composition may be formed into a solution and thereafter sprayed onto feed material. The method of administration of the compositions disclosed herein to the animal is considered to be within the skill of the artisan.

When used in combination with a feed material, the feed material can include corn, soybean meal, byproducts like distillers dried grains with solubles (DDGS), and vitamin/mineral supplement. Other feed materials can also be used.

Thus, in at least some embodiments, the effective amount of the composition comprising DFMs and optional exogenous feed enzymes is administered to an animal by supplementing a feed intended for the animal. As used herein, “supplementing,” refers to the action of incorporating the effective amount of bacteria provided herein directly into the feed intended for the animal. Thus, the animal, when feeding, ingests the bacteria provided herein.

### B. Methods for Preparing a Feed Additive Composition

Also provided herein are methods for preparing a feed additive composition comprising combining two or more of the DFMs disclosed herein. In some embodiments, the method includes combining two or more (such as any of 2, 3, or 4) of A. colihominis strain W1 (CBS 146120), or a strain having all of the identifying characteristics of A. colihominis strain W1, or a microbe having a 16S ribosomal RNA sequence displaying at least about 97.0% sequence similarity (such as any of about 97%, 97.5%, 98%, 98.5%, 99%, 99.5%, or 100% sequence similarity) to a 16S ribosomal RNA sequence of A. colihominis strain W1 (SEQ ID NO: 1); Anaerotruncus colihominis strain W2 (CBS 146122), or a strain having all of the identifying characteristics of A. colihominis strain W2, or a microbe having a 16S ribosomal RNA sequence displaying at least about 97.0% sequence similarity (such as any of about 97%, 97.5%, 98%, 98.5%, 99%, 99.5%, or 100% sequence similarity) to a 16S ribosomal RNA sequence of A. colihominis strain W2 (SEQ ID NO:2); Anaerotruncus colihominis strain W3 (CBS 146123), or a strain having all of the identifying characteristics of A. colihominis strain W3, or a microbe having a 16S ribosomal RNA sequence displaying at least about 97.0% sequence similarity (such as any of about 97%, 97.5%, 98%, 98.5%, 99%, 99.5%, or 100% sequence similarity) to a 16S ribosomal RNA sequence of A. colihominis strain W3 (SEQ ID NO: 3); and/or Anaerotruncus colihominis strain W4 (CBS 146121), or a strain having all of the identifying characteristics of A. colihominis strain W4, or a microbe having a 16S ribosomal RNA sequence displaying at least about 97.0% sequence similarity (such as any of about 97%, 97.5%, 98%, 98.5%, 99%, 99.5%, or 100% sequence similarity) to a 16S ribosomal RNA sequence of A. colihominis strain W4 (SEQ ID NO:4). In another embodiment, A. colihominis strains W1 and W2 are combined; A. colihominis strains W1 and W3 are combined; A. colihominis strains W1 and W4 are combined; A. colihominis strains W2 and W3 are combined; A. colihominis strains W2 and W4 are combined; A. colihominis strains W3 and W4 are combined; A. colihominis strains W1, W2, and W3 are combined; A. colihominis strains W1, W3, and W4 are combined; A. colihominis strains W2, W3, and W4 are combined; or A. colihominis strains W1, W2, W3, and W4 are combined.

In yet further embodiments, the method includes combining two or more (such as any of 2, 3, or 4) of Coprococcus sp. strain M1 (CBS 146125), or a strain having all of the identifying characteristics Coprococcus sp. strain M1, or a microbe having a 16S ribosomal RNA sequence displaying at least about 97.0% sequence similarity (such as any of about 97%, 97.5%, 98%, 98.5%, 99%, 99.5%, or 100% sequence similarity) to a 16S ribosomal RNA sequence of Coprococcus sp. strain M1 (SEQ ID NO:5); A. colihominis strain M2 (CBS 146119), or a strain having all of the identifying characteristics of A. colihominis strain M2, or a microbe having a 16S ribosomal RNA sequence displaying at least about 97.0% sequence similarity (such as any of about 97%, 97.5%, 98%, 98.5%, 99%, 99.5%, or 100% sequence similarity) to a 16S ribosomal RNA sequence of A. colihominis strain M2 (SEQ ID NO:6); C. lactatifermentans strain M3 (CBS 146124), or a strain having all of the identifying characteristics of C. lactatifermentans strain M3, or a microbe having a 16S ribosomal RNA sequence displaying at least about 97.0% sequence similarity (such as any of about 97%, 97.5%, 98%, 98.5%, 99%, 99.5%, or 100% sequence similarity) to a 16S ribosomal RNA sequence of C. lactatifermentans strain M3 (SEQ ID NO:7); and/or a strain having all of the identifying characteristics of a P. capillosus strain (such as P. capillosus strain M4) or a microbe having a 16S ribosomal RNA sequence displaying at least about 97.0% sequence similarity (such as any of about 97%, 97.5%, 98%, 98.5%, 99%, 99.5%, or 100% sequence similarity) to a 16S ribosomal RNA sequence of P. capillosus strain M4 (SEQ ID NO:8). In another embodiment, Coprococcus sp. strain M1 and A. colihominis strain M2 are combined; Coprococcus sp. strain M1 and C. lactatifermentans strain M3 are combined; Coprococcus sp. strain M1 and P. capillosus strain M4 are combined; A. colihominis strains M2 and C. lactatifermentans strain M3 are combined; A. colihominis strain M2 and P. capillosus strain M4 are combined; C. lactatifermentans strain M3 and P. capillosus strain M4 are combined; Coprococcus sp. strain M1, A. colihominis strain M2, and C. lactatifermentans strain M3 are combined; Coprococcus sp. strain M1, C. lactatifermentans strain M3, and P. capillosus strain M4 are combined; A. colihominis strain M2, C. lactatifermentans strain M3, and P. capillosus strain M4 are combined; or Coprococcus sp. strain M1, A. colihominis strain M2, C. lactatifermentans strain M3, and P. capillosus strain M4 are combined.

In additional embodiments, the method includes combining two or more (such as any of 2 or 3) of C. lactatifermentans strain 2F1 (CBS 146124), or a strain having all of the identifying characteristics C. lactatifermentans strain 2F1, or a microbe having a 16S ribosomal RNA sequence displaying at least about 97.0% sequence similarity (such as any of about 97%, 97.5%, 98%, 98.5%, 99%, 99.5%, or 100% sequence similarity) to a 16S ribosomal RNA sequence of C. lactatifermentans strain 2F1 (SEQ ID NO:9); L. salivarius strain 2F2 (CBS 146126), or a strain having all of the identifying characteristics of L. salivarius strain 2F2, or a microbe having a 16S ribosomal RNA sequence displaying at least about 97.0% sequence similarity (such as any of about 97%, 97.5%, 98%, 98.5%, 99%, 99.5%, or 100% sequence similarity) to a 16S ribosomal RNA sequence of L. salivarius strain 2F2 (SEQ ID NO:10); and/or L. reuteri strain 2F3 (CBS 145921), or a strain having all of the identifying characteristics of L. reuteri strain 2F3, or a microbe having a 16S ribosomal RNA sequence displaying at least about 97.0% sequence similarity (such as any of about 97%, 97.5%, 98%, 98.5%, 99%, 99.5%, or 100% sequence similarity) to a 16S ribosomal RNA sequence of L. reuteri strain 2F3 (SEQ ID NO: 11). In another embodiment, C. lactatifermentans strain 2F1 and L. salivarius strain 2F2 are combined; C. lactatifermentans strain 2F1 and L. reuteri strain 2F3 are combined; L. salivarius strain 2F2 and L. reuteri strain 2F3 are combined; or C. lactatifermentans strain 2F1, L. salivarius strain 2F2, and L. reuteri strain 2F3 are combined.

Additionally, the methods for preparing a feed additive composition can further include combining the feed additive composition with one or more of the exogenous enzymes disclosed herein (for example, one or more of a phytase, a protease, an amylase, a xylanase or a beta-glucanase). The method can additionally include a further step of packaging the feed additive composition for storage or transport.

### IV. Kits

Further provided herein are kits containing one or more of the DFMs (such as one or more of the multi-strain DFMs) disclosed herein. The kits can include one or more of (such as any of 1, 2, 3, 4, 5, 6, 7, 8, 9, 10, or 11) the strains provided herein inlcuding Anaerotruncus colihominis strain W1 (CBS 146120), Anaerotruncus colihominis strain W2 (CBS 146122), Anaerotruncus colihominis strain W3 (CBS 146123), Anaerotruncus colihominis strain W4 (CBS 146121), Coprococcus sp. strain M1 (CBS 146125), Anaerotruncus colihominis strain M2 (CBS 146119), Clostridium lactatifermentans strain M3 (CBS 146124), a Pseudoflavonifractor capillosus strain (such as P. capillosus strain M4), Clostridium lactatifermentans strain 2F1 (CBS 146124), Lactobacillus salivarius strain 2F2 (CBS 146126), and Lactobacillus reuteri strain 2F3 (CBS 145921) along with instructions for proper storage, maintenance, and use for administering to an animal to improve one or more performance metrics. In one embodiment, the kit can include strains W1, W2, W3, and/or W4. In another embodiment, the kit can include strains M1, M2, M3, and/or M4. In a further embodiment, the kit can include strains 2F1, 2F2, and/or 2F3. The kits can additionally include one or more of the exogenous enzymes disclosed herein (for example, one or more of a phytase, a protease, an amylase, a xylanase or a beta-glucanase).

The invention can be further understood by reference to the following examples, which are provided by way of illustration and are not meant to be limiting.

### EXAMPLES

### Example 1: Methods and Assays

In the following examples, various methods and assays were used as set forth below for ease in reading. Any deviations from the protocols provided below are indicated in the relevant sections.

Isolation of anaerobic microbes from chicken intestinal tracts: To isolate strict anaerobes, GIT samples dissected in the field were transferred into anaerobic transport media as quickly as possible. Strain isolation started once intestinal samples arrived at the lab. Ceca, ileum, jejunum or duodenum samples were dissected using sterile technique inside an anaerobic chamber. The digesta was discarded and mucosal-bound material was scraped using a loop. This material was then transferred into sterile media or buffer and serially diluted. Serial dilutions ranging from 10-1 to 10-6 were plated onto petri dishes or omni plates of various media types using plating beads. These plates were then incubated anaerobically until colonies became visible. For obligate anaerobes, brain heart infusion medium (BHI) was used.

Once colonies were visible on an agar plate, colonies were picked in the anaerobic chamber to liquid media in a 96 deep well plate. Some plates were initially picked into a small volume of liquid media (i.e. 200 μl) and then media added to 800 μl 1-4 days later to increase growth. Colony picking could be done at multiple time points for the same plate. For example, large colonies were picked on day 2, and then very small colonies or new colonies were picked at day 5.

Sample preparation and analysis of short-chain fatty acid production: Supernatants of bacterial cultures were evaluated for short-chain fatty acid (SCFA) production by GC methods. Cell cultures were centrifuged at 4° C. at 4100 RPM and 300 μl of the supernatant was removed by filtering through 0.2 um filter multi-well plate filtering unit (Pall Corporation, Product ID 8119). 150 μl of filtered supernatant was added to 150 μl of 0.5% H₃PO₄ solution, and filtered again through 0.2 μm filter plate. 140 μl of the prepared supernatant sample was transferred to a GC vial with volume inserts for GC-FID analysis.

GC separation of SCFAs on FFAP column has been previously reported (Zhao et al 2005). The GC instrumental method was modified to reduce sample run time to approximately 8 minutes. SCFA chromatographic analysis was carried out using Agilent 7890A GC system equipped with FID detector. A fused silica capillary column with a free-fatty acid phase was used (Agilent P/N 122-3232 DB-FFAP, 30 m×0.25 mm diameter, 0.25 μm film thickness). Helium gas was used as the carrier gas. The GC instrumental method was optimized to increase analysis time. The inlet temperature was 250° C. with 22.192 psi inlet pressure. The method was run under constant flow of 1.8 mL/min carrier gas flow rate. The initial oven temperature was 100° C., maintained for 0.3 min, raised to 240° C. at 17.5° C./min, with no hold time at final 240° C. temperature. The average gas velocity with these parameters was >42 cm/sec. The temperature of the FID was 300° C. The instrumental parameters reduced runtime to approximately 8 minutes. GC grade standards were used for external calibration.

Analysis of the microbial composition of mucosa region of the small intestines by 16S sequencing and netB qPCR: To evaluate the microbial composition of the small intestines, chicken swabs of the mucosa region of the small intestines was analyzed by 16S sequencing. Chicken gastro intestinal tract (GIT) is removed and separated into four sections: duodenum, jejunum, ileum and ceca. Each section is squeezed to remove the digesta contents and then cut longitudinally to expose the inner surface. Each section inner surface is swabbed with a FLOQSwab (Copan Mfgr, Murrieta, CA). The swab is added directly to a well of a 96 well Qiagen MagAttract PowerSoil kit (Qiagen, Hilden, Germany). The swabs were processed for bacterial DNA isolation as per the manufacturer's instructions using the KingFisher Flex automation platform. Isolated metagenomic DNA is then ready for NGS sample preparation

Metagenomic DNA purified from chicken GIT swabs was prepared for 16S community sequencing as follows: DNA is diluted 1:5 by adding 20 μl of molecular biology grade water to 5ul of purified DNA at 0.1-10 ng/μl. Then 2 μl of the diluted DNA was added to a PCR reaction along with 25 ul of ABI Universal TaqMan Reaction mix without UNG (ThermoFisher #4326614), 0.1ul each PCR primers at 100uM and 24.8ul of Molecular Biology Grade water for a total volume of 50 ul. The PCR primers were the Illumina-V4-515F-RJ: TCGTCGGCAGCGTCAGATGTGTATAAGAGACAGGTGCCAGCMGCCGCGGTAA (SEQ ID NO: 12) and Illumina-V4-806R-RJ: GTCTCGTGGGCTCGGAGATGTGTATAAGAGACAGGGACTACHVGGGTWTCTAAT (SEQ ID NO:13) were used. The PCR reactions were: 10 min at 95° C. followed by 35 cycles of 95° C. 15 sec+55° C. 30 sec+72° C. for 2 min. Amplified reactions were purified using Ampure XP Magnetic Beads (Beckman Coulter A63881) as per manufacturer's instructions using the Agilent Bravo Automated Robotic Workstation. 2 μl of each amplicon pool was then indexed in a second PCR reaction using the same conditions as above with Illumina XT Index Primers (Illumina XT v2.0 #FC-131-2001-2004) for 15 cycles. Indexed amplicons are then pooled and purified with AmPure XP Magnetic Beads on the Agilent Bravo Automated Robotic Workstation. Pooled, indexed amplicons were quantitated using the Kapa Illumina Library Quantification Kit (KAPA #KK4835) as per manufacturer's instructions. Purified, quantitated, indexed, pools were loaded on the Illumina MiSeq at a final concentration of 8 pM along with 15% Illumina PhiX (Illumina FC-110-3001). Sequencing was run for 2×250 Paired End cycles.

The 16S Amplicon data from Illumina Miseq sequencing were analyzed. Paired-end reads were first merged by Flash (Mogoc et al., Bioinformatics. 2011;27:2957-63). The forward and reverse primers were removed from the merged reads, and reads with overall quality score less than 20 were discarded by RDP Initial Process tool (Fish et al., Front Microbiol. 2013;4:291). This step also removes reads originated from chicken mitochondria due to their length shorter than 200 bp. In the next step reads were assigned to bacterial and archaeal taxonomy by RDP Classifier (Wang et al., Appl Environ Microbiol. 73(16):5261-5267). The reads passed the above quality processing steps were clustered at 98% by CD-HIT (Li & Godzik, Bioinformatics, 2006; 22:1658-9) to obtain Operational Taxonomic Units (OTU). The representative sequence from each OTU was assigned to the closest species by RDP pairwise alignment tool (Fish et al., Front Microbiol. 2013; 4:291) against a vetted 16S reference database containing mostly 16S genes from type strains and public genomes. The relative abundance of an OTU in a sample was the fraction of reads assigned to that OTU. An OTU was assigned to a species if it has at least 98% identity to that species in the reference database. The average abundance of a species in a consortium in small intestine was calculated as the average relative abundance of that species from DUO, JEJ and ILL samples of each treatment.

The same DNA preparations were also quantified for netB gene using qPCR. NetB is an important virulence factor for C. perfringens. Each assay was run using 1.5 μl of DNA along with 10 μl of TaqMan Universal Master Mix, 0.2 μl of 100 uM Forward and Reverse Primers, 0.05 μl of TaqMan Probe, and 9.55 μl Molecular Biology Grade water. The qPCR reaction conditions were as follows: 10 min at 95° C.+40 cycles of 95° C. 15 sec+60° C. 60 sec on the ABI Quant Studio qPCR instrument. Sample data was quantified using genomic DNA from a netB positive C. perfringens. Primer and probe sequences used are in Table 5 below:

TABLE 5
qPCR Primers and Probes

| | | | Sequence |
|---|---|---|---|
| Target | Primer Name | Direction | (5′ to 3′) |
| Total | 16S-T1-1369F | for | CGGTGAATACGTT |
| Bacteria | | | CYCGG (SEQ ID |
| 16S | | | NO: 14) |
| | 16S-T1-1492R | rev | GGWTACCTTGTTA |
| | | | CGACTT (SEQ |
| | | | ID NO: 15) |
| | 16S-T1-1389T | probe | CTTGTACACACCG |
| | | | CCCGTC (SEQ |
| | | | ID NO: 16) |
| C. | CPerfl65F | for | CGCATAACGTTGA |
| perfringens | | | AAGATGG (SEQ |
| | | | ID NO: 17) |
| | CPerf269R | rev | CCTTGGTAGGCCG |
| | | | TTACCC (SEQ |
| | | | ID NO: 18) |
| | CPerfl87T | probe | TCATCATTCAACC |
| | | | AAAGGAGCAATCC |
| | | | (SEQ ID NO: |
| | | | 19) |
| netB gene | NetB-RJ-Fwd | Fwd | TGGTGCTGGAATA |
| | | | AATGCTTCAT |
| | | | (SEQ ID NO: |
| | | | 20) |
| | NetB-RJ-Rev | Rev | TGCATCATCTTTT |
| | | | CTTTGAATTGTTC |
| | | | (SEQ ID NO: |
| | | | 21) |
| | NetB-RJ-MGB | Probe | ATACTATAAGCTA |
| | | | TGAACAACC |
| | | | (SEQ ID NO: |
| | | | 22) |

### Example 2: Short-Chain Fatty Acid Production by Obligate Anaerobic Bacteria From Broiler Chicken

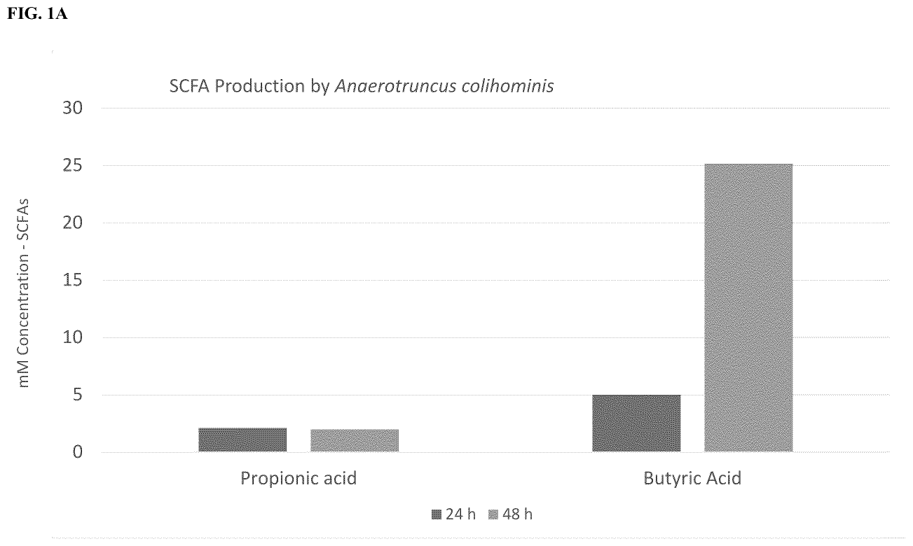

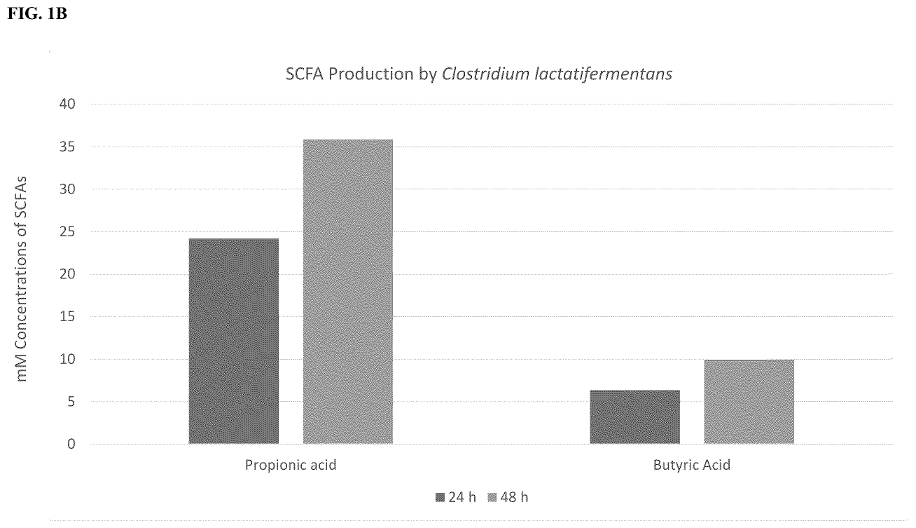

Based on the method described in Example 1, a variety of obligate anaerobes were identified which produce SCFAs, including both Gram-positive and Gram-negative bacteria. Table 6 lists the production of butyrate and propionate from non-limiting examples of bacteria isolated from chicken intestinal tracts. Most of them produced butyrate as the major SCFA (FIG. 1A). One strain, Clostridium lactatifermentans M3, on the other hand, produced propionate as the major SCFA (FIG. 1B).

TABLE 6
Short-chain fatty acid production
(mM) by certain obligate anaerobes

| Strains | Butyrate | Propionate |
|---|---|---|
| Clostridium MSTE9 2A1 | 7.68 | ND |
| Clostridium MSTE9 2A2 | 12.04 | ND |
| Clostridium MSTE9 2A3 | 6.14 | |
| Clostridium nexile Z1 | 10.76 | ND |
| Clostridium nexile Z2 | 16.21 | ND |
| Clostridium nexile Z3 | 8.95 | ND |
| Clostridium nexile Z4 | 16.31 | ND |
| Coprococcus sp. M1 | 25.37 | ND |
| Anaerotruncus colihominis M2 | 25.37 | ND |
| Clostridium lactatifermentans M3 | 9.22 | 54.63 |
| Pseudoflavonifractor capillosus M4 | 22.00 | ND |
| Anaerotruncus colihominis W1 | 17.3 | ND |
| Anaerotruncus colihominis M2 | 15.67 | ND |
| Anaerotruncus colihominis M3 | 21.01 | ND |
| Anaerotruncus colihominis M4 | 8.00 | ND |

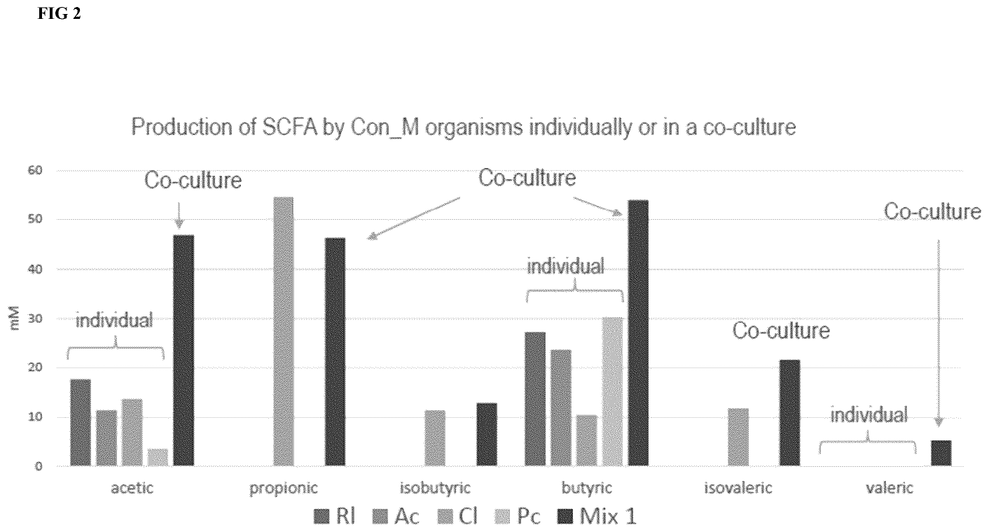

To evaluate the potential interaction of SCFA producing microbes, four SCFA producing microbes were mixed as c-culture in the BHI medium. These four organisms were Coprococcus sp. M1, Anaerotruncus colihominis M2, Clostridium lactatifermentans M3, and Pseudoflavonifractor capillosus M4 as listed in Table 6. The SCFA contents were analyzed in the supernatant. As shown in Table 7, the amount of propionate from the co-culture was proportional to the amount generated by Clostridium lactatifermentans as measured in the individual culture. However, the amount of butyrate production in the co-culture was higher than the amount generated by any individual strain, suggesting an additive effect. For the valerate production, it was not detected in any of the individual strain cultured alone. The co-culture, on the other hand, produced valerate, suggesting synergistic effect (FIG. 2). Valerate has been demonstrated to have efficacy for reducing the incidence of necrotic enteritis (Poult Sci. 2018 Jul. 1;97 (7):2303-2311).

TABLE 7
Comparison of SCFA profiles by individual culture and mixed culture.

| Strains | Butyrate | Propionate | Acetate | Valerate |
|---|---|---|---|---|
| Coprococcus sp. M1 | 25.37 | ND | 16.69 | ND |
| Anaerotruncus colihominis M2 | 25.37 | ND | 10.59 | ND |
| Clostridium lactatifermentans M3 | 9.22 | 54.63 | 11.11 | ND |
| Pseudoflavonifractor capillosus M4 | 22 | ND | 5.53 | ND |
| Mixture of above 4 strains | 51.52 | 42.75 | 45.27 | 5.01 |

### Example 3: Evaluation of SCFA Producing Bacteria for The Prevention of Necrotic Enteritis Through Animal Studies

A challenged diseased model for broiler chicken has been used extensively (Front Microbiol. 2016, 7:1416; J Anim Sci Biotechnol. 2018, 9:25; Poult Sci. 2018 November 18). In this experiment, chickens at day 9 were first challenged with live 1×Eimeria vaccine (ADVENT® coccidiosis vaccine, Huvepharma, Inc., Lincoln, NE 68528). Seventeen (17) birds were in each of the experimental and control groups. The vaccine was diluted in water and each chicken received 1ml orally. At day 11, each chicken received 1 ml of pathogen cocktail orally. The pathogen cocktail consisted of five Clostridium perfringens strains isolated from diseased tissues. All strains contained the netB and Tpel genes based on the genome analysis. These strains were grown individually in cooked meat medium (Sigma) overnight and the fresh cultures were mixed in equal volume in a glove box to make up the cocktail. The cocktail was used the same day to induce necrotic enteritis.

With this disease model, DEM candidates were evaluated for their efficacy against C. perfringens infection. For anaerobic bacteria DEM candidates were grown in BHI medium under anaerobic conditions. and the fresh culture were mixed in equal volume inside the glovebox to make up a specific consortium for the animal trial. The mixed cell suspension for each consortium was aliquoted into serum bottles under anaerobic conditions inside a glove box. The serum bottles were stored at 80 oC and thawed before use. The DFM consortia were fed to each chicken daily by gavage starting at day 1. At day 15, chicken intestines were collected for histopathology analysis and the mucosa samples were used for DNA isolation as described in Example 1.

The DFM consortia and their strain composition are listed in Table 8. For the first animal trial, consortia K, and N consisted of anaerobic microbes that do not produce SCFAs. Only consortium M consisted of strains that can produce SCFAs. For the control group of chicken, no DFM candidates were used. For the second animal trial, all consortia consisted microbes that can produce SCFAs.

TABLE 8
List of consortia and their strain compositions used for animal trials

| | |
|---|---|
| Consortia | Components |

Trial 1

| | |
|---|---|
| K | Clostridium glycyrrhizinilyticum K1, Anaerofilum agile K2, Bacteroides dorei K3 |
| M | Coprococcus sp. M1, Anaerotruncus colihominis M2, Clostridium lactatifermentans M3, |
| | Pseudoflavonifractor capillosus M4 |
| N | Bacteroides cellulosilyticus N1, Bacteroides fragilis N2, Parabacteroides distasatonis N3 |

Trial 2

| | |
|---|---|
| 2A | Clostridium sp. MSTE9 2A1, Clostridium sp. MSTE9 2A2, Clostridium sp. MSTE9 2A3 |
| Z | Clostridium sp. MSTE9 2A1, Clostridium sp. MSTE9 2K2, Clostridium sp. MSTE9 2A3 |
| W | Anaerotruncus colihominis W1, Anaerotruncus colihominis M2, Anaerotruncus |
| | colihominis M3, Anaerotruncus colihominis M4 |

The abundance of C. perfringens based on 16S analysis using samples from day 15 as described in Example 1 is summarized in Table 9. Consortia K and N included different anaerobic bacteria (Table 8). These microbes did not produce SCFAs. Based on 16S analysis, chicken feed with these two consortia showed similar abundance of C. perfringens as the chicken in the disease model group. The result from qPCR analysis of the netB gene showed a similar trend. This suggests that these two DFM consortia did not show efficacy against the expansion of the pathogen in the animal trial. Consistent with this observation, histopathology analysis showed the presence of necrotic enteritis in these samples. On the other hand, the abundance of C. perfringens was much lower in consortium M. This consortium consisted of four different anaerobic strains that produced SCFAs as described in Example 2. Interestingly, as demonstrated in Example 2, when these four strains were mixed as co-culture, the SCFAs profile was different in comparison to when these strains are cultured individually. Histopathology indicated no sign of necrotic enteritis. This suggests that consortium M had efficacy in the prevention of C. perfringens infection.

To further investigate whether other SCFA producing microbes isolated from chicken would have similar effect on reducing the incidence of necrotic enteritis, consortia 2A, Z, and W were designed and used in another animal trial (Table 8; Trial 2). Based on 16S analysis and netB qPCR (Table 9), consortia, 2A and Z, showed high abundance of C. perfringens in the small intestines. Histopathology analysis indicated necrotic enteritis just like the disease model. This result suggests that these two SCFA-producing consortia had no impact. On the other hand, a new consortium, W, was found to be efficacious against the pathogen. This consortium consisted of isolates from the Anaerotruncus colihominis species.

TABLE 9
Results of animal trials

| | | | |
|---|---|---|---|
| Consortium | CP Abundance¹ | netB qPCR² | Necrotic Enteritis |

Trial 1

| | | | |
|---|---|---|---|
| Model | 0.173 | 0.204 | Positive |
| K | 0.507 | 0.828 | Positive |
| M | 0.004 | 0.001 | Negative |
| N | 0.281 | 0.314 | Positive |

Trial 2

| | | | |
|---|---|---|---|
| Model | 0.197 | 0.195 | Positive |
| 2A | 0.785 | 1.447 | Positive |
| Z | 0.688 | 1.749 | Positive |
| W | 0.002 | 0.04 | Negative |

¹CP Abundance = 16S CP sequence reads/Total 16S Sequence reads (Example 1)
²netB qPCR = qPCR netB Copy number/qPCR Total 16S

### Example 4: A Combination SCFA Producing Microbe with Lactobacillus Species

To investigate a combination of a SCFA producing microbe with other potential DFM candidates that do not produce SCFAs, another animal trial was carried out with consortium 2F (Table 10). Consortium 2F consisted of C. Lactatifermentans M3, a SCFA producer from consortium M (Table 6), and two different isolates of Lactobacillus.

TABLE 10
Combined SCFA producer and Lactobacillus
consortium and strain composition used for trial

| | |
|---|---|
| Consortia | Components |

Trial 3

| | |
|---|---|
| 2F | C. Lactatifermentans M3, |
| | L. salivarius U1, L. reuteri S1 |

As shown in Table 11, feeding consortium to the animal greatly reduced the CP counts based on both 16S analysis and netB PCR quantification as compared to the model where no DEM candidates were used. In addition, no necrotic enteritis was detected in the small intestines of chicken treated with 2F consortium. The result indicated that a combination of a SCFA producer and Lactobacillus species as present in the 2F consortium had the efficacy of preventing the CP infection.

TABLE 11
Results of animal trials

| | | | |
|---|---|---|---|
| Consortium | CP Abundance¹ | netB qPCR² | Necrotic Enteritis |

Trial 3

| | | | |
|---|---|---|---|
| Model | 0.427 | 0.466 | Positive |
| 2F | 0.001 | 0.066 | Negative |

SEQUENCES
A. colihominis strain W1 16S rRNA
(SEQ ID NO: 1)
CAAAGGAGCAATCCGCTGAAAGATGGGCTCGCGTCCGATTAGCCAGTTGGCGGGGTAACGG
CCCACCAAAGCGACGATCGGTAGCCGGACTGAGAGGTTGAACGGCCACATTGGGACTGAGA
CACGGCCCAGACTCCTACGGGAGGCAGCAGTGGGGGATATTGCACAATGGGCGAAAGCCTG
ATGCAGCGACGCCGCGTGAGGGAAGACGGTCTTCGGATTGTAAACCTCTGTCTTTGGGGAA
GAAAATGACGGTACCCAAAGAGGAAGCTCCGGCTAACTACGTGCCAGCAGCCGCGGTAATA
CGTAGGGAGCAAGCGTTGTCCGGAATTACTGGGTGTAAAGGGAGCGTAGGCGGGATGGCAA
GTAGAATGTTAAATCCATCGGCTCAACCGGTGGCTGCGTTCTAAACTGCCGTTCTTGAGTG
AAGTAGAGGCAGGCGGAATTCCTAGTGTAGCGGTGAAATGCGTAGATATTAGGAGGAACAC
CAGTGGCGAAGGCGGCCTGCTGGGCTTTAACTGACGCTGAGGCTCGAAAGCGTGGGGAGCA
AACAGGATTAGATACCCTGGTAGTCCACGCCGTAAACGATGATTACTAGGTGTGGGGGGAC
TGACCCCTTCCGTGCCGCAGTTAACACAATAAGTAATCCACCTGGGGAGTACGGCCGCAAG
GTTGAAACTCAAAGGAATTGACGGGGGCCCGCACAAGCAGTGGAGTATGTGGTTTAATTCG
AAGCAACGCGAAGAACCTTACCAGGTCTTGACATCGGATGCATAGCCTGGAGACAGGTGAA
GCCCTTCGGGGCATCCAGACAGGTGGTGCATGGTTGTCGTCAGCTCGTGTCGTGAGATGTT
GGGTTAAGTCCCGCAACGAGCGCAACCCTTATTATTAGTTGCTACGCAAGAGCACTCTAAT
GAGACTGCCGTTGACAAAACGGAGGAAGGTGGGGATGACGTCAAATCATCATGCCCCTTAT
GACCTGGGCTACACACGTACTACAATGGCACTTAAACAGAGGGCGGCGACACCGCGAGGTG
AAGCGAATCCCGAAAAAGTGTCTCAGTTCAGATTGCAGGCTGCAACCCGCCTGCATGAAGT
CGGAATTGCTAGTAATCGCGGATCAGCATGCCGCGGTGAATACGTTCCCGGGCCTTGTACA
CACCGCCCGTCACACCATGGGAGTCGGTAACACCCGAAGCCAGTAGCCTAACCGCAAGGGG
GGCGCTGTCGAAGGTGGGATTGATGACTGGGGTGAAGTCGTAACAAGGTAGCCGTATCGGA
AGGTGCGGCTGGATCACCTCCTTT
A. colihominis strain W2 16S rRNA
(SEQ ID NO: 2)
CAAAGAGTTTGATCCTGGCTCAGGACGAACGCTGGCGGCGCGCCTAACACATGCAAGTCGA
ACGGGGCTTACATTTTGAAGTTTTCGGATGGACGAATGTAAGCTTAGTGGCGGACGGGTGA
GTAACACGTGAGCAACCTGCCTTTCAGAGGGGGATAACAGCCGGAAACGGCTGCTAATACC
GCATAATGTTGCGGGGGCACATGCCCCTGCAACCAAAGGAGCAATCCGCTGAAAGATGGGC
TCGCGTCCGATTAGCCAGTTGGCGGGGTAACGGCCCACCAAAGCGACGATCGGTAGCCGGA
CTGAGAGGTTGAACGGCCACATTGGGACTGAGACACGGCCCAGACTCCTACGGGAGGCAGC
AGTGGGGGATATTGCACAATGGGCGAAAGCCTGATGCAGCGACGCCGCGTGAGGGAAGACG
GTCTTCGGATTGTAAACCTCTGTCTTTGGGGAAGAAAATGACGGTACCCAAAGAGGAAGCT
CCGGCTAACTACGTGCCAGCAGCCGCGGTAATACGTAGGGAGCAAGCGTTGTCCGGAATTA
CTGGGTGTAAAGGGAGCGTAGGCGGGATGGCAAGTAGAATGTTAAATCCATCGGCTCAACC
GGTGGCTGCGTTCTAAACTGCCGTTCTTGAGTGAAGTAGAGGCAGGCGGAATTCCTAGTGT
AGCGGTGAAATGCGTAGATATTAGGAGGAACACCAGTGGCGAAGGCGGCCTGCTGGGCTTT
AACTGACGCTGAGGCTCGAAAGCGTGGGGAGCAAACAGGATTAGATACCCTGGTAGTCCAC
GCCGTAAACGATGATTACTAGGTGTGGGGGGACTGACCCCTTCCGTGCCGCAGTTAACACA
ATAAGTAATCCACCTGGGGAGTACGGCCGCAAGGTTGAAACTCAAAGGAATTGACGGGGGC
CCGCACAAGCAGTGGAGTATGTGGTTTAATTCGAAGCAACGCGAAGAACCTTACCAGGTCT
TGACATCGGATGCATAGCCTGGAGACAGGTGAAGCCCTTCGGGGCATCCAGACAGGTGGTG
CATGGTTGTCGTCAGCTCGTGTCGTGAGATGTTGGGTTAAGTCCCGCAACGAGCGCAACCC
TTATTATTAGTTGCTACGCAAGAGCACTCTAATGAGACTGCCGTTGACAAAACGGAGGAAG
GTGGGGATGACGTCAAATCATCATGCCCCTTATGACCTGGGCTACACACGTACTACAATGG
CACTTAAACAGAGGGCGGCGACACCGCGAGGTGAAGCGAATCCCGAAAAAGTGTCTCAGTT
CAGATTGCAGGCTGCAACCCGCCTGCATGAAGTCGGAATTGCTAGTAATCGCGGATCAGCA
TGCCGCGGTGAATACGTTCCCGGGCCTTGTACACACCGCCCGTCACACCATGGGAGTCGGT
AACACCCGAAGCCAGTAGCCTAACCGCAAGGGGGGCGCTGTCGAAGGTGGGATTGATGACT
GGGGTGAAGTCGTAACAAGGTAGCCGTATCGGAAGGTGCGGCTGGATCACCTCCTTT
A. colihominis strain W3 16S rRNA
(SEQ ID NO: 3)
CAAAGGAGCAATCCGCTGAAAGATGGGCTCGCGTCCGATTAGCCAGTTGGCGGGGTAACGG
CCCACCAAAGCGACGATCGGTAGCCGGACTGAGAGGTTGAACGGCCACATTGGGACTGAGA
CACGGCCCAGACTCCTACGGGAGGCAGCAGTGGGGGATATTGCACAATGGGCGAAAGCCTG
ATGCAGCGACGCCGCGTGAGGGAAGACGGTCTTCGGATTGTAAACCTCTGTCTTTGGGGAA
GAAAATGACGGTACCCAAAGAGGAAGCTCCGGCTAACTACGTGCCAGCAGCCGCGGTAATA
CGTAGGGAGCAAGCGTTGTCCGGAATTACTGGGTGTAAAGGGAGCGTAGGCGGGATGGCAA
GTAGAATGTTAAATCCATCGGCTCAACCGGTGGCTGCGTTCTAAACTGCCGTTCTTGAGTG
AAGTAGAGGCAGGCGGAATTCCTAGTGTAGCGGTGAAATGCGTAGATATTAGGAGGAACAC
CAGTGGCGAAGGCGGCCTGCTGGGCTTTAACTGACGCTGAGGCTCGAAAGCGTGGGGAGCA
AACAGGATTAGATACCCTGGTAGTCCACGCCGTAAACGATGATTACTAGGTGTGGGGGGAC
TGACCCCTTCCGTGCCGCAGTTAACACAATAAGTAATCCACCTGGGGAGTACGGCCGCAAG
GTTGAAACTCAAAGGAATTGACGGGGGCCCGCACAAGCAGTGGAGTATGTGGTTTAATTCG
AAGCAACGCGAAGAACCTTACCAGGTCTTGACATCGGATGCATAGCCTGGAGACAGGTGAA
GCCCTTCGGGGCATCCAGACAGGTGGTGCATGGTTGTCGTCAGCTCGTGTCGTGAGATGTT
GGGTTAAGTCCCGCAACGAGCGCAACCCTTATTATTAGTTGCTACGCAAGAGCACTCTAAT
GAGACTGCCGTTGACAAAACGGAGGAAGGTGGGGATGACGTCAAATCATCATGCCCCTTAT
GACCTGGGCTACACACGTACTACAATGGCACTTAAACAGAGGGCGGCGACACCGCGAGGTG
AAGCGAATCCCGAAAAAGTGTCTCAGTTCAGATTGCAGGCTGCAACCCGCCTGCATGAAGT
CGGAATTGCTAGTAATCGCGGATCAGCATGCCGCGGTGAATACGTTCCCGGGCCTTGTACA
CACCGCCCGTCACACCATGGGAGTCGGTAACACCCGAAGCCAGTAGCCTAACCGCAAGGGG
GGCGCTGTCGAAGGTGGGATTGATGACTGGGGTGAAGTCGTAACAAGGTAGCCGTATCGGA
AGGTGCGGCTGGATCACCTCCTTT
A. colihominis strain W4 16S rRNA
(SEQ ID NO: 4)
CAAAGAGTTTGATCCTGGCTCAGGACGAACGCTGGCGGCGCGCCTAACACATGCAAGTCGA
ACGGGGCTTACATTTTGAAGTTTTCGGATGGACGAATGTAAGCTTAGTGGCGGACGGGTGA
GTAACACGTGAGCAACCTGCCTTTCAGAGGGGGATAACAGCCGGAAACGGCTGCTAATACC
GCATAATGTTGCGGGGGCACATGCCCCTGCAACCAAAGGAGCAATCCGCTGAAAGATGGGC
TCGCGTCCGATTAGCCAGTTGGCGGGGTAACGGCCCACCAAAGCGACGATCGGTAGCCGGA
CTGAGAGGTTGAACGGCCACATTGGGACTGAGACACGGCCCAGACTCCTACGGGAGGCAGC
AGTGGGGGATATTGCACAATGGGCGAAAGCCTGATGCAGCGACGCCGCGTGAGGGAAGACG
GTCTTCGGATTGTAAACCTCTGTCTTTGGGGAAGAAAATGACGGTACCCAAAGAGGAAGCT
CCGGCTAACTACGTGCCAGCAGCCGCGGTAATACGTAGGGAGCAAGCGTTGTCCGGAATTA
CTGGGTGTAAAGGGAGCGTAGGCGGGATGGCAAGTAGAATGTTAAATCCATCGGCTCAACC
GGTGGCTGCGTTCTAAACTGCCGTTCTTGAGTGAAGTAGAGGCAGGCGGAATTCCTAGTGT
AGCGGTGAAATGCGTAGATATTAGGAGGAACACCAGTGGCGAAGGCGGCCTGCTGGGCTTT
AACTGACGCTGAGGCTCGAAAGCGTGGGGAGCAAACAGGATTAGATACCCTGGTAGTCCAC
GCCGTAAACGATGATTACTAGGTGTGGGGGGACTGACCCCTTCCGTGCCGCAGTTAACACA
ATAAGTAATCCACCTGGGGAGTACGGCCGCAAGGTTGAAACTCAAAGGAATTGACGGGGGC
CCGCACAAGCAGTGGAGTATGTGGTTTAATTCGAAGCAACGCGAAGAACCTTACCAGGTCT
TGACATCGGATGCATAGCCTGGAGACAGGTGAAGCCCTTCGGGGCATCCAGACAGGTGGTG
CATGGTTGTCGTCAGCTCGTGTCGTGAGATGTTGGGTTAAGTCCCGCAACGAGCGCAACCC
TTATTATTAGTTGCTACGCAAGAGCACTCTAATGAGACTGCCGTTGACAAAACGGAGGAAG
GTGGGGATGACGTCAAATCATCATGCCCCTTATGACCTGGGCTACACACGTACTACAATGG
CACTAAAACAGAGGGCGGCGACACCGCGAGGTGAAGCGAATCCCGAAAAAGTGTCTCAGTT
CAGATTGCAGGCTGCAACCCGCCTGCATGAAGTCGGAATTGCTAGTAATCGCGGATCAGCA
TGCCGCGGTGAATACGTTCCCGGGCCTTGTACACACCGCCCGTCACACCATGGGAGTCGGT
AACACCCGAAGCCAGTAGCCTAACCGCAAGGGGGGCGCTGTCGAAGGTGGGATTGATGACT
GGGGTGAAGTCGTAACAAGGTAGCCGTATCGGAAGGTGCGGCTGGATCACCTCCTTT
Coprococcus sp. strain M1 16S rRNA
(SEQ ID NO: 5)
AACGAGAGTTTGATCCTGGCTCAGGATGAACGCTGGCGGCGTGCCTAACACATGCAAGTCG
AACGAAGCACTTACTTCCAAATCTTCGGAAGAGGAGGTACTTGACTGAGTGGCGGACGGGT
GAGTAACGCGTGGGGAACCTGCCCCGTACCGGGGGATAACAGTCAGAAATGACTGCTAATA
CCGCATAAGCGCACGAAGGCGCATGCTTTTGTGTGAAAAACTCCGGTGGTACGGGATGGTC
CCGCGTCTGATTAGCCAGTTGGCGGGGTAACGGCCCACCAAAGCGACGATCAGTAGCCGGC
CTGAGAGGGTGGACGGCCACATTGGGACTGAGACACGGCCCAGACTCCTACGGGAGGCAGC
AGTGGGGAATATTGCACAATGGGGGAAACCCTGATGCAGCGACGCCGCGTGAGCGAAGAAG
TATTTCGGTATGTAAAGCTCTGTCAGCAGGGAAGAAAATGACGGTACCTGACCAAGAAGCA
CCGGCTAAATACGTGCCAGCAGCCGCGGTAATACGTATGGTGCAAGCGTTATCCGGATTTA
CTGGGTGTAAAGGGAGCGTAGACGGAGGGGCAAGTCTGAAGTGAAAGCCCGGGGCCCAACC
CCGGGACTGCTTTGGAAACTGTCCGTCTGGAGTGCCGGAGAGGTAAGCGGAATTCCCAGTG
TAGCGGTGAAATGCGTAGATATTGGGAGGAACACCAGTGGCGAAGGCGGCTTACTGGACGG
TCACTGACGTTGAGGCTCGAAAGCGTGGGGAGCAAACAGGATTAGATACCCTGGTAGTCCA
CGCCGTAAACGATGACTACTAGGTGTCGGGTGGCAGAGCCATTCGGTGCCGCAGCCAACGC
AGTAAGTAGTCCACCTGGGGAGTACGTTCGCAAGAATGAAACTCAAAGGAATTGACGGGGA
CCCGCACAAGCGGTGGAGCATGTGGTTTAATTCGAAGCAACGCGAAGAACCTTACCTGGCC
TTGACATCCCCCTGACCGGCGCGTAATGGTGCCTTTCCTTCGGGACAGGGGAGACAGGTGG
TGCATGGTTGTCGTCAGCTCGTGTCGTGAGATGTTGGGTTAAGTCCCGCAACGAGCGCAAC
CCTTATCTTCAGTAGCCAGCACGCAGAGGTGGGCACTCTGGAGAGACTGCCAGGGACAACC
TGGAGGAAGGTGGGGATGACGTCAAATCATCATGCCCCTTATGGCCAGGGCTACACACGTG
CTACAATGGCGTAAACAGAGGGAAGCGAGCCCGCGAGGGGGAGCAAATCCCAAAAATAACG
TCCCAGTTCGGACTGCAGGCTGCAACCCGCCTGCACGAAGCTGGAATCGCTAGTAATCGCG
AATCAGCATGTCGCGGTGAATACGTTCCCGGGTCTTGTACACACCGCCCGTCACACCATGG
GAGTCGGTAACGCCCGAAGTCAGTGACCCAACCTCCGGGAGGGAGCTGCCGAAGGCGGGAC
CGGTAACTGGGGTGAAGTCGTAACAAGGTAGCCGTATCGGAAGGTGCGGCTGGATCACCTC
CTTT
A. colihominis strain M2 16S rRNA
(SEQ ID NO: 6)
CAAAGAGTTTGATCCTGGCTCAGGACGAACGCTGGCGGCGCGCCTAACACATGCAAGTCGA
ACGGGGCTTACATTTTGAAGTTTTCGGATGGACGAATGTAAGCTTAGTGGCGGACGGGTGA
GTAACACGTGAGCAACCTGCCTTTCAGAGGGGGATAACAGCCGGAAACGGCTGCTAATACC
GCATAATGTTGCGGGGGCACATGCCCCTGCAACCAAAGGAGCAATCCGCTGAAAGATGGGC
TCGCGTCCGATTAGCCAGTTGGCGGGGTAACGGCCCACCAAAGCGACGATCGGTAGCCGGA
CTGAGAGGTTGAACGGCCACATTGGGACTGAGACACGGCCCAGACTCCTACGGGAGGCAGC
AGTGGGGGATATTGCACAATGGGCGAAAGCCTGATGCAGCGACGCCGCGTGAGGGAAGACG
GTCTTCGGATTGTAAACCTCTGTCTTTGGGGAAGAAAATGACGGTACCCAAAGAGGAAGCT
CCGGCTAACTACGTGCCAGCAGCCGCGGTAATACGTAGGGAGCAAGCGTTGTCCGGAATTA
CTGGGTGTAAAGGGAGCGTAGGCGGGATGGCAAGTAGAATGTTAAATCCATCGGCTCAACC
GGTGGCTGCGTTCTAAACTGCCGTTCTTGAGTGAAGTAGAGGCAGGCGGAATTCCTAGTGT
AGCGGTGAAATGCGTAGATATTAGGAGGAACACCAGTGGCGAAGGCGGCCTGCTGGGCTTT
AACTGACGCTGAGGCTCGAAAGCGTGGGGAGCAAACAGGATTAGATACCCTGGTAGTCCAC
GCCGTAAACGATGATTACTAGGTGTGGGGGGACTGACCCCTTCCGTGCCGCAGTTAACACA
ATAAGTAATCCACCTGGGGAGTACGGCCGCAAGGTTGAAACTCAAAGGAATTGACGGGGGC
CCGCACAAGCAGTGGAGTATGTGGTTTAATTCGAAGCAACGCGAAGAACCTTACCAGGTCT
TGACATCGGATGCATAGCCTGGAGACAGGTGAAGCCCTTCGGGGCATCCAGACAGGTGGTG
CATGGTTGTCGTCAGCTCGTGTCGTGAGATGTTGGGTTAAGTCCCGCAACGAGCGCAACCC
TTATTATTAGTTGCTACGCAAGAGCACTCTAATGAGACTGCCGTTGACAAAACGGAGGAAG
GTGGGGATGACGTCAAATCATCATGCCCCTTATGACCTGGGCTACACACGTACTACAATGG
CACTAAAACAGAGGGCGGCGACACCGCGAGGTGAAGCGAATCCCGAAAAAGTGTCTCAGTT
CAGATTGCAGGCTGCAACCCGCCTGCATGAAGTCGGAATTGCTAGTAATCGCGGATCAGCA
TGCCGCGGTGAATACGTTCCCGGGCCTTGTACACACCGCCCGTCACACCATGGGAGTCGGT
AACACCCGAAGCCAGTAGCCTAACCGCAAGGGGGGCGCTGTCGAAGGTGGGATTGATGACT
GGGGTGAAGTCGTAACAAGGTAGCCGTATCGGAAGGTGCGGCTGGATCACCTCCTTT
C. lactatifermentans strain M3 16S rRNA
(SEQ ID NO: 7)
CTTAGTGGCGGACGGGTGAGTAACGTGTGGGCAACCTGCCCTGTACTGGGGAATAATCATT
GGAAACGATGACTAATACCGCATGTGGTCCTCGGAAGGCATCTTCTGAGGAAGAAAGGATT
TATTCGGTACAGGATGGGCCCGCATCTGATTAGCTAGTTGGTGAGATAACAGCCCACCAAG
GCGACGATCAGTAGCCGACCTGAGAGGGTGATCGGCCACATTGGGACTGAGACACGGCCCA
AACTCCTACGGGAGGCAGCAGTGGGGAATATTGCACAATGGGCGAAAGCCTGATGCAGCAA
CGCCGCGTGAAGGATGAAGGGTTTCGGCTCGTAAACTTCTATCAATAGGGAAGAAAGAAAT
GACGGTACCTAAATAAGAAGCCCCGGCTAACTACGTGCCAGCAGCCGCGGTAATACGTAGG
GGGCAAGCGTTATCCGGAATTACTGGGTGTAAAGGGAGCGTAGGCGGCATGGTAAGCCAGA
TGTGAAAGCCTTGGGCTTAACCCGAGGATTGCATTTGGAACTATCAAGCTAGAGTACAGGA
GAGGAAAGCGGAATTCCTAGTGTAGCGGTGAAATGCGTAGATATTAGGAAGAACACCAGTG
GCGAAGGCGGCTTTCTGGACTGAAACTGACGCTGAGGCTCGAAAGCGTGGGGAGCAAACAG
GATTAGATACCCTGGTAGTCCACGCCGTAAACGATGAGTGCTAGGTGTCGGGGAGGAATCC
TCGGTGCCGCAGCTAACGCAATAAGCACTCCACCTGGGGAGTACGACCGCAAGGTTGAAAC
TCAAAGGAATTGACGGGGGCCCGCACAAGCGGTGGAGCATGTGGTTTAATTCGAAGCAACG
CGAAGAACCTTACCAAGGCTTGACATCCCGATGACCGCTCTAGAGATAGAGCTTCTCTTCG
GAGCATCGGTGACAGGTGGTGCATGGTTGTCGTCAGCTCGTGTCGTGAGATGTTGGGTTAA
GTCCCGCAACGAGCGCAACCCTTATCTTCAGTAGCCATCATTTAGTTGGGCACTCTGGAGA
GACTGCCGTGGATAACACGGAGGAAGGTGGGGATGACGTCAAATCATCATGCCCCTTATGT
CTTGGGCTACACACGTGCTACAATGGCTGGTAACAAAGTGACGCAAAACGGCGACGTTAAG
CAAATCACAAAAACCCAGTCCCAGTTCGGATTGTAGTCTGCAACTCGACTACATGAAGCTG
GAATCGCTAGTAATCGCGAATCAGCATGTCGCGGTGAATACGTTCCCGGGCCTTGTACACA
CCGCCCGTCACACCATGGGAGTTGGAAGCACCCGAAGTCGGTGACCTAACCGTAAGGAAGG
AGCCGCCGAAGGTGAAGCCAGTGACTGGGGTGAAGTCGTAACAAGGTAGCCGTATCGGAAG
GTGCGGCTGGATCACCTCCTTT
P. capillosus strain M4 16S rRNA
(SEQ ID NO: 8)
TATTGAGAGTTTGATCCTGGCTCAGGATGAACGCTGGCGGCGTGCTTAACACATGCAAGTC
GAACGGAGAGCTAATGACAGAGGATTCGTCCAATGGATTTAGTTTCTTAGTGGCGGACGGG
TGAGTAACGCGTGAGGAACCTGCCTCGGAGTGGGGAATAACACAACGAAAGCTGTGCTAAT
ACCGCATGATGCAGCTGGGTCGCATGACTCTGGCTGCCAAAGATTTATCGCTCTGAGATGG
CCTCGCGTCTGATTAGCTGGTTGGCGGGGTAACGGCCCACCAAGGCGACGATCAGTAGCCG
GACTGAGAGGTTGGCCGGCCACATTGGGACTGAGACACGGCCCAGACTCCTACGGGAGGCA
GCAGTGGGGAATATTGGGCAATGGGCGCAAGCCTGACCCAGCAACGCCGCGTGAAGGAAGA
AGGCCCTCGGGTTGTAAACTTCTTTTGTCAGGGACGAAGCAAGTGACGGTACCTGACGAAT
AAGCCACGGCTAACTACGTGCCAGCAGCCGCGGTAATACGTAGGTGGCAAGCGTTATCCGG
ATTTACTGGGTGTAAAGGGCGTGTAGGCGGGATTGCAAGTCAGATGTGAAAACCACGGGCT
CAACCTGTGGCCTGCATTTGAAACTGCAGTTCTTGAGTACTGGAGAGGCAGACGGAATTCC
TAGTGTAGCGGTGAAATGCGTAGATATTAGGAGGAACACCAGTGGCGAAGGCGGTCTGCTG
GACAGCAACTGACGCTGAGGCGCGAAAGCGTGGGGAGCAAACAGGATTAGATACCCTGGTA
GTCCACGCTGTAAACGATGGATACTAGGTGTGGGGGGTCTGACCCCCTCCGTGCCGCAGTT
AACACAATAAGTATCCCACCTGGGGAGTACGATCGCAAGGTTGAAACTCAAAGGAATTGAC
GGGGGCCCGCACAAGCGGTGGAGTATGTGGTTTAATTCGAAGCAACGCGAAGAACCTTACC
AGGGCTTGACATCCCGATGACCGGACTAGAGATAGTCTTTTCTCTTCGGAGACATCGGTGA
CAGGTGGTGCATGGTTGTCGTCAGCTCGTGTCGTGAGATGTTGGGTTAAGTCCCGCAACGA
GCGCAACCCCTATTGTTAGTTGCTACGCAAGAGCACTCTAGCGAGACTGCCGTTGACAAAA
CGGAGGAAGGCGGGGACGACGTCAAATCATCATGCCCCTTATGTCCTGGGCCACACACGTA
CTACAATGGTGGTTAACAGAGGGAGGCAATACCGCGAGGTGGAGCAAACCCCTAAAAGCCA
TCCCAGTTCGGATTGCAGGCTGCAACCCGCCTGCATGAAGTTGGAATCGCTAGTAATCGCG
GATCAGCATGCCGCGGTGAATACGTTCCCGGGCCTTGTACACACCGCCCGTCACACCATGA
GAGTCGGGAACACCCGAAGTCCGTAGCCTAACCGCAAGGGGGGCGCGGCCGAAGGTGGGTT
CGATAATTGGGGTGAAGTCGTAACAAGGTAGCCGTATCGGAAGGTGCGGCTGGATCACCTC
CTTT
C. lactatifermentans strain 2F1 16S rRNA
(SEQ ID NO: 9)
CTTAGTGGCGGACGGGTGAGTAACGTGTGGGCAACCTGCCCTGTACTGGGGAATAATCATT
GGAAACGATGACTAATACCGCATGTGGTCCTCGGAAGGCATCTTCTGAGGAAGAAAGGATT
TATTCGGTACAGGATGGGCCCGCATCTGATTAGCTAGTTGGTGAGATAACAGCCCACCAAG
GCGACGATCAGTAGCCGACCTGAGAGGGTGATCGGCCACATTGGGACTGAGACACGGCCCA
AACTCCTACGGGAGGCAGCAGTGGGGAATATTGCACAATGGGCGAAAGCCTGATGCAGCAA
CGCCGCGTGAAGGATGAAGGGTTTCGGCTCGTAAACTTCTATCAATAGGGAAGAAAGAAAT
GACGGTACCTAAATAAGAAGCCCCGGCTAACTACGTGCCAGCAGCCGCGGTAATACGTAGG
GGGCAAGCGTTATCCGGAATTACTGGGTGTAAAGGGAGCGTAGGCGGCATGGTAAGCCAGA
TGTGAAAGCCTTGGGCTTAACCCGAGGATTGCATTTGGAACTATCAAGCTAGAGTACAGGA
GAGGAAAGCGGAATTCCTAGTGTAGCGGTGAAATGCGTAGATATTAGGAAGAACACCAGTG
GCGAAGGCGGCTTTCTGGACTGAAACTGACGCTGAGGCTCGAAAGCGTGGGGAGCAAACAG
GATTAGATACCCTGGTAGTCCACGCCGTAAACGATGAGTGCTAGGTGTCGGGGAGGAATCC
TCGGTGCCGCAGCTAACGCAATAAGCACTCCACCTGGGGAGTACGACCGCAAGGTTGAAAC
TCAAAGGAATTGACGGGGGCCCGCACAAGCGGTGGAGCATGTGGTTTAATTCGAAGCAACG
CGAAGAACCTTACCAAGGCTTGACATCCCGATGACCGCTCTAGAGATAGAGCTTCTCTTCG
GAGCATCGGTGACAGGTGGTGCATGGTTGTCGTCAGCTCGTGTCGTGAGATGTTGGGTTAA
GTCCCGCAACGAGCGCAACCCTTATCTTCAGTAGCCATCATTTAGTTGGGCACTCTGGAGA
GACTGCCGTGGATAACACGGAGGAAGGTGGGGATGACGTCAAATCATCATGCCCCTTATGT
CTTGGGCTACACACGTGCTACAATGGCTGGTAACAAAGTGACGCAAAACGGCGACGTTAAG
CAAATCACAAAAACCCAGTCCCAGTTCGGATTGTAGTCTGCAACTCGACTACATGAAGCTG
GAATCGCTAGTAATCGCGAATCAGCATGTCGCGGTGAATACGTTCCCGGGCCTTGTACACA
CCGCCCGTCACACCATGGGAGTTGGAAGCACCCGAAGTCGGTGACCTAACCGTAAGGAAGG
AGCCGCCGAAGGTGAAGCCAGTGACTGGGGTGAAGTCGTAACAAGGTAGCCGTATCGGAAG
GTGCGGCTGGATCACCTCCTTT
Lactobacillus salivarius strain 2F2 16S rRNA
(SEQ ID NO: 10)
AATGAGAGTTTGATCCTGGCTCAGGACGAACGCTGGCGGCGTGCCTAATACATGCAAGTCG
AACGAAACTTTCTTACACCGAATGCTTGCATTCACCGTAAGAAGTTGAGTGGCGGACGGGT
GAGTAACACGTGGGTAACCTGCCTAAAAGAAGGGGATAACACTTGGAAACAGGTGCTAATA
CCGTATATCTCTAAGGATCGCATGATCCTTAGATGAAAGATGGTTCTGCTATCGCTTTTAG
ATGGACCCGCGGCGTATTAACTAGTTGGTGGGGTAACGGCCTACCAAGGTGATGATACGTA
GCCGAACTGAGAGGTTGATCGGCCACATTGGGACTGAGACACGGCCCAAACTCCTACGGGA
GGCAGCAGTAGGGAATCTTCCACAATGGACGCAAGTCTGATGGAGCAACGCCGCGTGAGTG
AAGAAGGTCTTCGGATCGTAAAACTCTGTTGTTAGAGAAGAACACGAGTGAGAGTAACTGT
TCATTCGATGACGGTATCTAACCAGCAAGTCACGGCTAACTACGTGCCAGCAGCCGCGGTA
ATACGTAGGTGGCAAGCGTTGTCCGGATTTATTGGGCGTAAAGGGAACGCAGGCGGTCTTT
TAAGTCTGATGTGAAAGCCTTCGGCTTAACCGGAGTAGTGCATTGGAAACTGGAAGACTTG
AGTGCAGAAGAGGAGAGTGGAACTCCATGTGTAGCGGTGAAATGCGTAGATATATGGAAGA
ACACCAGTGGCGAAAGCGGCTCTCTGGTCTGTAACTGACGCTGAGGTTCGAAAGCGTGGGT
AGCAAACAGGATTAGATACCCTGGTAGTCCACGCCGTAAACGATGAATGCTAGGTGTTGGA
GGGTTTCCGCCCTTCAGTGCCGCAGCTAACGCAATAAGCATTCCGCCTGGGGAGTACGACC
GCAAGGTTGAAACTCAAAGGAATTGACGGGGGCCCGCACAAGCGGTGGAGCATGTGGTTTA
ATTCGAAGCAACGCGAAGAACCTTACCAGGTCTTGACATCCTTTGACCACCTAAGAGATTA
GGTTTTCCCTTCGGGGACAAAGTGACAGGTGGTGCATGGCTGTCGTCAGCTCGTGTCGTGA
GATGTTGGGTTAAGTCCCGCAACGAGCGCAACCCTTGTTGTCAGTTGCCAGCATTAAGTTG
GGCACTCTGGCGAGACTGCCGGTGACAAACCGGAGGAAGGTGGGGACGACGTCAAGTCATC
ATGCCCCTTATGACCTGGGCTACACACGTGCTACAATGGACGGTACAACGAGTCGCAAGAC
CGCGAGGTTTAGCTAATCTCTTAAAGCCGTTCTCAGTTCGGATTGTAGGCTGCAACTCGCC
TACATGAAGTCGGAATCGCTAGTAATCGCGAATCAGCATGTCGCGGTGAATACGTTCCCGG
GCCTTGTACACACCGCCCGTCACACCATGAGAGTTTGTAACACCCAAAGCCGGTGGGGTAA
CCGCAAGGAGCCAGCCGTCTAAGGTGGGACAGATGATTGGGGTGAAGTCGTAACAAGGTAG
CCGTAGGAGAACCTGCGGCTGGATCACCTCCTTT
Lactobacillus reuteri strain 2F3 16S rRNA
(SEQ ID NO: 11)
GTGACGGTATCCAACCAGAAAGTCACGGCTAACTACGTGCCAGCAGCCGCGGTAATACGTA
GGTGGCAAGCGTTATCCGGATTTATTGGGCGTAAAGCGAGCGCAGGCGGTTGCTTAGGTCT
GATGTGAAAGCCTTCGGCTTAACCGAAGAAGTGCATCGGAAACCGGGCGACTTGAGTGCAG
AAGAGGACAGTGGAACTCCATGTGTAGCGGTGGAATGCGTAGATATATGGAAGAACACCAG
TGGCGAAGGCGGCTGTCTGGTCTGCAACTGACGCTGAGGCTCGAAAGCATGGGTAGCGAAC
AGGATTAGATACCCTGGTAGTCCATGCCGTAAACGATGAGTGCTAGGTGTTGGAGGGTTTC
CGCCCTTCAGTGCCGGAGCTAACGCATTAAGCACTCCGCCTGGGGAGTACGACCGCAAGGT
TGAAACTCAAAGGAATTGACGGGGGCCCGCACAAGCGGTGGAGCATGTGGTTTAATTCGAA
GCTACGCGAAGAACCTTACCAGGTCTTGACATCTTGCGCTAACCTTAGAGATAAGGCGTTC
CCTTCGGGGACGCAATGACAGGTGGTGCATGGTCGTCGTCAGCTCGTGTCGTGAGATGTTG
GGTTAAGTCCCGCAACGAGCGCAACCCTTGTTACTAGTTGCCAGCATTAAGTTGGGCACTC
TAGTGAGACTGCCGGTGACAAACCGGAGGAAGGTGGGGACGACGTCAGATCATCATGCCCC
TTATGACCTGGGCTACACACGTGCTACAATGGACGGTACAACGAGTCGCAAGCTCGCGAGA
GTAAGCTAATCTCTTAAAGCCGTTCTCAGTTCGGACTGTAGGCTGCAACTCGCCTACACGA
AGTCGGAATCGCTAGTAATCGCGGATCAGCATGCCGCGGTGAATACGTTCCCGGGCCTTGT
ACACACCGCCCGTCACACCATGGGAGTTTGTAACGCCCAAAGTCGGTGGCCTAACCTTTAT
GGAGGGAGCCGCCTAAGGCGGGACAGATGACTGGGGTGAAGTCGTAACAAGGTAGCCGTAG
GAGAACCTGCGGCTGGATCACCTCCTTT

We claim:

1. A feed additive composition comprising a direct fed microbial (DFM) comprising one or more biologically pure bacterial strains of Anaerotruncus colihominis.

2. The feed additive composition of claim 1, comprising four strains of A. colihominis.

3. The feed additive composition of claim 1 or claim 2, comprising a) a bacterial strain having a 16S ribosomal RNA sequence displaying at least 97.0% sequence similarity to a 16S ribosomal RNA sequence of a Anaerotruncus colihominis strain W1 deposited at Westerdijk Fungal Biodiversity Institute (WFDB) under number CBS 146120; b) a bacterial strain having a 16S ribosomal RNA sequence displaying at least 97.0% sequence similarity to a 16S ribosomal RNA sequence of a Anaerotruncus colihominis strain W2 deposited at Westerdijk Fungal Biodiversity Institute (WFDB) under number CBS 146122; c) a bacterial strain having a 16S ribosomal RNA sequence displaying at least 97.0% sequence similarity to a 16S ribosomal RNA sequence of a Anaerotruncus colihominis strain W3 deposited at Westerdijk Fungal Biodiversity Institute (WFDB) under number CBS 146123; and d) a bacterial strain having a 16S ribosomal RNA sequence displaying at least 97.0% sequence similarity to a 16S ribosomal RNA sequence of a Anaerotruncus colihominis strain W4 deposited at Westerdijk Fungal Biodiversity Institute (WFDB) under number CBS 146121.

4. The feed additive composition of any one of claims 1-3, wherein the composition comprises a) A. colihominis strain W1 (CBS 146120) or a live strain having all of the identifying characteristics of A. colihominis strain W1 (CBS 146120); b) A. colihominis strain W2 (CBS 146122) or a live strain having all of the identifying characteristics of A. colihominis strain W2 (CBS 146122); c) A. colihominis strain W3 (CBS 146123) or a live strain having all of the identifying characteristics of A. colihominis strain W3 (CBS 146123); and d) A. colihominis strain W4 (CBS 146121) or a live strain having all of the identifying characteristics of A. colihominis strain W4 (CBS 146121) either (i) alone; or (ii) in combination with a culture supernatant derived from each of these strains.

5. The feed additive composition of claim 1, further comprising biologically pure bacterial strains of a) a Coprococcus sp.; b) Clostridium lactatifermentans; and c) Pseudoflavonifractor capillosus.

6. The feed additive composition of claim 5, comprising a) a bacterial strain having a 16S ribosomal RNA sequence displaying at least 97.0% sequence similarity to a 16S ribosomal RNA sequence of a Coprococcus sp. strain M1 deposited at Westerdijk Fungal Biodiversity Institute (WFDB) under number CBS 146125; b) a bacterial strain having a 16S ribosomal RNA sequence displaying at least 97.0% sequence similarity to a 16S ribosomal RNA sequence of a Anaerotruncus colihominis strain M2 deposited at Westerdijk Fungal Biodiversity Institute (WFDB) under number CBS 146119; c) a bacterial strain having a 16S ribosomal RNA sequence displaying at least 97.0% sequence similarity to a 16S ribosomal RNA sequence of a Clostridium lactatifermentans strain M3 deposited at Westerdijk Fungal Biodiversity Institute (WFDB) under number CBS 146124; and d) a bacterial strain having a 16S ribosomal RNA sequence displaying at least 97.0% sequence similarity to a 16S ribosomal RNA sequence of a Pseudoflavonifractor capillosus strain M4.

7. The feed additive composition of claim 5 or 6, wherein the composition comprises a) Coprococcus sp. strain M1 (CBS 146125) or a live strain having all of the identifying characteristics of Coprococcus sp. strain M1 (CBS 146125); b) A. colihominis strain M2 (CBS 146119) or a live strain having all of the identifying characteristics of A. colihominis strain M2 (CBS 146119); c) C. lactatifermentans strain M3 (CBS 146124) or a live strain having all of the identifying characteristics of C. lactatifermentans strain M3 (CBS 146124); and d) a P. capillosus strain either (i) alone; or (ii) in combination with a culture supernatant derived from each of these strains.

8. A feed additive composition comprising a direct fed microbial (DFM) comprising one or more biologically pure bacterial strains of a short chain fatty acid (SCFA)-producing bacterial strain and one or more biologically pure lactic acid producing bacterial strains.

9. The feed additive composition of claim 8, comprising a) a biologically pure strain of Clostridium lactatifermentans; and b) two biologically pure lactic acid producing bacterial strains.

10. The feed additive composition of claim 8 or claim 9, wherein the two biologically pure lactic acid producing bacterial strains are Lactobacillus salivarius and Lactobacillus reuteri.

11. The feed additive composition of any one of claims 8-10, comprising a) a bacterial strain having a 16S ribosomal RNA sequence displaying at least 97.0% sequence similarity to a 16S ribosomal RNA sequence of a Clostridium lactatifermentans strain 2F1 deposited at Westerdijk Fungal Biodiversity Institute (WFDB) under number 146124; b) a bacterial strain having a 16S ribosomal RNA sequence displaying at least 97.0% sequence similarity to a 16S ribosomal RNA sequence of a Lactobacillus salivarius strain 2F2 deposited at Westerdijk Fungal Biodiversity Institute (WFDB) under number CBS 146126; and c) a bacterial strain having a 16S ribosomal RNA sequence displaying at least 97.0% sequence similarity to a 16S ribosomal RNA sequence of a Lactobacillus reuteri strain 2F3 deposited at Westerdijk Fungal Biodiversity Institute (WFDB) under number CBS 145921.

12. The feed additive composition of any one of claims 8-11, wherein the composition comprises a) C. lactatifermentans strain 2F1 (CBS 146124) or a live strain having all of the identifying characteristics of C. lactatifermentans strain 2F1 (CBS 146124); b) L. salivarius strain 2F2 (CBS 146126) or a live strain having all of the identifying characteristics of L. salivarius strain 2F2 (CBS 146126); and c) L. reuteri strain 2F3 (CBS 145921) or a live strain having all of the identifying characteristics of L. reuteri strain 2F3 (CBS 145921) either (i) alone; or (ii) in combination with a culture supernatant derived from each of these strains.

13. The feed additive composition of claim 3 or claim 4, wherein a) the 16S ribosomal RNA sequence of Anaerotruncus colihominis strain W1 comprises the nucleotide sequence of SEQ ID NO: 1; b) the 16S ribosomal RNA sequence of Anaerotruncus colihominis strain W2 comprises the nucleotide sequence of SEQ ID NO:2; c) the 16S ribosomal RNA sequence of Anaerotruncus colihominis strain W3 comprises the nucleotide sequence of SEQ ID NO:3; and d) the 16S ribosomal RNA sequence of Anaerotruncus colihominis strain W4 comprises the nucleotide sequence of SEQ ID NO:4.

14. The feed additive composition of claim 6 or claim 7, wherein a) the 16S ribosomal RNA sequence of Coprococcus sp. strain M1 comprises the nucleotide sequence of SEQ ID NO:5; b) the 16S ribosomal RNA sequence of Anaerotruncus colihominis strain M2 comprises the nucleotide sequence of SEQ ID NO:6; c) the 16S ribosomal RNA sequence of Clostridium lactatifermentans strain M3 comprises the nucleotide sequence of SEQ ID NO:7; and d) the 16S ribosomal RNA sequence of Pseudoflavonifractor capillosus strain M4 comprises the nucleotide sequence of SEQ ID NO:8.

15. The feed additive composition of claim 11 or claim 12, wherein a) the 16S ribosomal RNA sequence of Clostridium lactatifermentans strain 2F1 comprises the nucleotide sequence of SEQ ID NO:9; b) the 16S ribosomal RNA sequence of Lactobacillus salivarius strain 2F2 comprises the nucleotide sequence of SEQ ID NO:10; and c) the 16S ribosomal RNA sequence of Lactobacillus reuteri strain 2F3 comprises the nucleotide sequence of SEQ ID NO:11.

16. The feed additive composition of any one of claims 1-15, wherein the composition produces one or more short chain fatty acids selected from the group consisting of butyrate, isobutyrate, propionate, acetate, isovalerate, and valerate.

17. The feed additive composition of any one of claims 1-16, further comprising one or more enzymes.

18. The feed additive composition of claim 17, wherein the one or more enzymes are selected from the group consisting of a phytase, a protease, an amylase, a xylanase and a beta-glucanase.

19. The feed additive composition of any one of claims 1-18, wherein each strain is present at a concentration of at least about 1×10³ CFU/g feed additive composition to at least about 1×10⁹ CFU/g feed additive composition.

20. The feed additive composition of any one of claims 1-19, wherein the composition inhibits at least one pathogen selected from avian pathogenic Salmonella sp., Escherichia coli, Clostridium perfringens and Enterobacteriaceae in a gastrointestinal tract of a bird having ingested an effective amount of said direct fed microbial composition.

21. A premix comprising the feed additive composition of any one of claims 1-20 and at least one mineral and/or at least one vitamin.

22. A feed comprising the feed additive compositions of any one of claims 1-20 or the premix of claim 21.

23. A kit comprising a) the feed additive composition of any one of claims 1-20; and b) written instructions for administration to an animal.

24. The kit of claim 23, further comprising one or more enzymes.

25. The kit of claim 24, wherein the one or more enzymes are selected from the group consisting of a phytase, a protease, an amylase, a xylanase and a beta-glucanase.

26. A method for improving one or more metrics in an animal selected from the group consisting of increased bodyweight gain, intestinal health status, decreased feed conversion ratio (FCR), improved gut barrier integrity, reduced mortality, reduced pathogen infection, and reduced pathogen shedding in feces comprising administering an effective amount of the feed additive composition of any one of claims 1-20, the premix of claim 21, or the feed of claim 22 to the animal, thereby improving the one or more metrics in the animal.

27. The method of claim 26, wherein the feed additive composition increases one or more of the acetate, isobutyrate, butyrate, isovalerate, and/or valerate content of the gastrointestinal tract of the animal.

28. The method of claim 26 or claim 27, wherein the feed additive composition increases the butyrate content of the gastrointestinal tract of the animal.

29. The method of any one of claims 26-28, wherein the pathogen is one or more of Clostridium perfringens, Campylobacter jejuni, Enterobacteriaceae, a Salmonela sp., and/or Escherichia coli.

30. The method of any one of claims 26-29, wherein the method further treats, prevents, or decreases incidence of necrotic enteritis.

31. The method of any one of claims 26-30, wherein the animal is a domesticated bird.

32. The method of claim 31, wherein the domesticated bird is selected from the group consisting of chickens, turkeys, ducks, geese, quail, emus, ostriches, and pheasant.

33. The method of claim 32, wherein the chicken is a broiler or a layer.

34. A method for preparing a feed additive composition comprising combining a) a bacterial strain having a 16S ribosomal RNA sequence displaying at least 97.0% sequence similarity to a 16S ribosomal RNA sequence of a Anaerotruncus colihominis strain W1 deposited at CBS under number CBS 146120; b) a bacterial strain having a 16S ribosomal RNA sequence displaying at least 97.0% s sequence similarity to a 16S ribosomal RNA sequence of a Anaerotruncus colihominis strain W2 deposited at CBS under number CBS 146122; c) a bacterial strain having a 16S ribosomal RNA sequence displaying at least 97.0% sequence similarity to a 16S ribosomal RNA sequence of a Anaerotruncus colihominis strain W3 deposited at CBS under number CBS 146123; and d) a bacterial strain having a 16S ribosomal RNA sequence displaying at least 97.0% sequence similarity to a 16S ribosomal RNA sequence of a Anaerotruncus colihominis strain W4 deposited at CBS under number CBS 146121.

35. The method of claim 34, wherein a) the A. colihominis strain W1 is an A. colihominis strain W1 (CBS 146120) or a live strain having all of the identifying characteristics of A. colihominis strain W1 (CBS 146120); b) the A. colihominis strain W2 is an A. colihominis strain W2 (CBS 146122) or a live strain having all of the identifying characteristics of A. colihominis strain W2 (CBS 146122); c) the A. colihominis strain W3 is an A. colihominis strain W3 (CBS 146123) or a live strain having all of the identifying characteristics of A. colihominis strain W3 (CBS 146123); and d) the A. colihominis strain W4 is an A. colihominis strain W4 (CBS 146121) or a live strain having all of the identifying characteristics of A. colihominis strain W4 (CBS 146121) either (i) alone; or (ii) in combination with a culture supernatant derived from each of these strains.

36. A method for preparing a feed additive composition comprising combining a) a bacterial strain having a 16S ribosomal RNA sequence displaying at least 97.0% sequence similarity to a 16S ribosomal RNA sequence of a Coprococcus sp. strain M1 deposited at CBS under number CBS 146125; b) a bacterial strain having a 16S ribosomal RNA sequence displaying at least 97.0% sequence similarity to a 16S ribosomal RNA sequence of a Anaerotruncus colihominis strain M2 deposited at CBS under number CBS 146119; c) a bacterial strain having a 16S ribosomal RNA sequence displaying at least 97.0% sequence similarity to a 16S ribosomal RNA sequence of a Clostridium lactatifermentans strain M3 deposited at CBS under number CBS 146124; and d) a bacterial strain having a 16S ribosomal RNA sequence displaying at least 97.0% sequence similarity to a 16S ribosomal RNA sequence of a Pseudoflavonifractor capillosus strain M4.

37. The method of claim 36, wherein the a) Coprococcus sp. strain M1 is an Coprococcus sp. strain M1 (CBS 146125) or a live strain having all of the identifying characteristics of Coprococcus sp. strain M1 (CBS 146125); b) the A. colihominis strain M2 is an A. colihominis strain M2 (CBS 146119) or a live strain having all of the identifying characteristics of A. colihominis strain M2 (CBS 146119); and c) the C. lactatifermentans strain M3 is a C. lactatifermentans strain M3 (CBS 146124) or a live strain having all of the identifying characteristics of C. lactatifermentans strain M3 (CBS 146124) either (i) alone; or (ii) in combination with a culture supernatant derived from each of these strains.

38. A method for preparing a feed additive composition comprising combining a) a bacterial strain having a 16S ribosomal RNA sequence displaying at least 97.0% sequence similarity to a 16S ribosomal RNA sequence of a Clostridium lactatifermentans strain 2F1 deposited at CBS under number CBS 146124; b) a bacterial strain having a 16S ribosomal RNA sequence displaying at least 97.0% s sequence similarity to a 16S ribosomal RNA sequence of a Lactobacillus salivarius strain 2F2 deposited at CBS under number CBS 146126; and c) a bacterial strain having a 16S ribosomal RNA sequence displaying at least 97.0% sequence similarity to a 16S ribosomal RNA sequence of a Lactobacillus reuteri strain 2F3 deposited at CBS under number CBS 145921.

39. The method of claim 34, wherein a) the C. lactatifermentans strain 2F1 is an C. lactatifermentans strain W1 (CBS 146124) or a live strain having all of the identifying characteristics of C. lactatifermentans strain 2F1 (CBS 146124); b) the L. salivarius strain 2F2 is an L. salivarius strain 2F2 (CBS 146126) or a live strain having all of the identifying characteristics of L. salivarius strain 2F2 (CBS 146126); and c) the L. reuteri strain 2F3 is an L. reuteri strain 2F3 (CBS 145921) or a live strain having all of the identifying characteristics of L. reuteri strain 2F3 (CBS 145921) either (i) alone; or (ii) in combination with a culture supernatant derived from each of these strains.

40. The method of any one of claims 34-39, further comprising combining one or more enzyme with the feed additive composition.

41. The method of claim 40, wherein the one or more enzymes are selected from the group consisting of a phytase, a protease, an amylase, a xylanase and a beta-glucanase.

42. The method of any one of claims 34-41, wherein at least about 1×10³ CFU/g feed additive composition to at least about 1×10⁹ CFU/g feed additive composition is combined to form the feed additive composition.

43. The method of any one of claims 34-42, further comprising packaging the feed additive composition.

44. A method for preparing a premix comprising combining the feed additive composition of any one of claims 1-20 with at least one mineral and/or at least one vitamin.

45. The method of claim 44, further comprising packaging the premix.


SEQUENCE LISTING (22 sequences)

SEQ ID NO: 1 - DNA, 1305 bp, Anaerotruncus colihominis
  source 1..1305: mol_type = other DNA
caaaggagcaatccgctgaaagatgggctcgcgtccgattagccagttggcggggtaacggcccaccaaagcgacgatcggtagccggactgagaggttgaacggccacattgggactgagacacggcccagactcctacgggaggcagcagtgggggatattgcacaatgggcgaaagcctgatgcagcgacgccgcgtgagggaagacggtcttcggattgtaaacctctgtctttggggaagaaaatgacggtacccaaagaggaagctccggctaactacgtgccagcagccgcggtaatacgtagggagcaagcgttgtccggaattactgggtgtaaagggagcgtaggcgggatggcaagtagaatgttaaatccatcggctcaaccggtggctgcgttctaaactgccgttcttgagtgaagtagaggcaggcggaattcctagtgtagcggtgaaatgcgtagatattaggaggaacaccagtggcgaaggcggcctgctgggctttaactgacgctgaggctcgaaagcgtggggagcaaacaggattagataccctggtagtccacgccgtaaacgatgattactaggtgtggggggactgaccccttccgtgccgcagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatcggatgcatagcctggagacaggtgaagcccttcggggcatccagacaggtggtgcatggttgtcgtcagctcgtgtcgtgagatgttgggttaagtcccgcaacgagcgcaacccttattattagttgctacgcaagagcactctaatgagactgccgttgacaaaacggaggaaggtggggatgacgtcaaatcatcatgccccttatgacctgggctacacacgtactacaatggcacttaaacagagggcggcgacaccgcgaggtgaagcgaatcccgaaaaagtgtctcagttcagattgcaggctgcaacccgcctgcatgaagtcggaattgctagtaatcgcggatcagcatgccgcggtgaatacgttcccgggccttgtacacaccgcccgtcacaccatgggagtcggtaacacccgaagccagtagcctaaccgcaaggggggcgctgtcgaaggtgggattgatgactggggtgaagtcgtaacaaggtagccgtatcggaaggtgcggctggatcacctccttt

SEQ ID NO: 2 - DNA, 1521 bp, Anaerotruncus colihominis
  source 1..1521: mol_type = other DNA
caaagagtttgatcctggctcaggacgaacgctggcggcgcgcctaacacatgcaagtcgaacggggcttacattttgaagttttcggatggacgaatgtaagcttagtggcggacgggtgagtaacacgtgagcaacctgcctttcagagggggataacagccggaaacggctgctaataccgcataatgttgcgggggcacatgcccctgcaaccaaaggagcaatccgctgaaagatgggctcgcgtccgattagccagttggcggggtaacggcccaccaaagcgacgatcggtagccggactgagaggttgaacggccacattgggactgagacacggcccagactcctacgggaggcagcagtgggggatattgcacaatgggcgaaagcctgatgcagcgacgccgcgtgagggaagacggtcttcggattgtaaacctctgtctttggggaagaaaatgacggtacccaaagaggaagctccggctaactacgtgccagcagccgcggtaatacgtagggagcaagcgttgtccggaattactgggtgtaaagggagcgtaggcgggatggcaagtagaatgttaaatccatcggctcaaccggtggctgcgttctaaactgccgttcttgagtgaagtagaggcaggcggaattcctagtgtagcggtgaaatgcgtagatattaggaggaacaccagtggcgaaggcggcctgctgggctttaactgacgctgaggctcgaaagcgtggggagcaaacaggattagataccctggtagtccacgccgtaaacgatgattactaggtgtggggggactgaccccttccgtgccgcagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatcggatgcatagcctggagacaggtgaagcccttcggggcatccagacaggtggtgcatggttgtcgtcagctcgtgtcgtgagatgttgggttaagtcccgcaacgagcgcaacccttattattagttgctacgcaagagcactctaatgagactgccgttgacaaaacggaggaaggtggggatgacgtcaaatcatcatgccccttatgacctgggctacacacgtactacaatggcacttaaacagagggcggcgacaccgcgaggtgaagcgaatcccgaaaaagtgtctcagttcagattgcaggctgcaacccgcctgcatgaagtcggaattgctagtaatcgcggatcagcatgccgcggtgaatacgttcccgggccttgtacacaccgcccgtcacaccatgggagtcggtaacacccgaagccagtagcctaaccgcaaggggggcgctgtcgaaggtgggattgatgactggggtgaagtcgtaacaaggtagccgtatcggaaggtgcggctggatcacctccttt

SEQ ID NO: 3 - DNA, 1305 bp, Anaerotruncus colihominis; identical to SEQ ID NO: 1
  source 1..1305: mol_type = other DNA

SEQ ID NO: 4 - DNA, 1521 bp, Anaerotruncus colihominis
  source 1..1521: mol_type = other DNA
caaagagtttgatcctggctcaggacgaacgctggcggcgcgcctaacacatgcaagtcgaacggggcttacattttgaagttttcggatggacgaatgtaagcttagtggcggacgggtgagtaacacgtgagcaacctgcctttcagagggggataacagccggaaacggctgctaataccgcataatgttgcgggggcacatgcccctgcaaccaaaggagcaatccgctgaaagatgggctcgcgtccgattagccagttggcggggtaacggcccaccaaagcgacgatcggtagccggactgagaggttgaacggccacattgggactgagacacggcccagactcctacgggaggcagcagtgggggatattgcacaatgggcgaaagcctgatgcagcgacgccgcgtgagggaagacggtcttcggattgtaaacctctgtctttggggaagaaaatgacggtacccaaagaggaagctccggctaactacgtgccagcagccgcggtaatacgtagggagcaagcgttgtccggaattactgggtgtaaagggagcgtaggcgggatggcaagtagaatgttaaatccatcggctcaaccggtggctgcgttctaaactgccgttcttgagtgaagtagaggcaggcggaattcctagtgtagcggtgaaatgcgtagatattaggaggaacaccagtggcgaaggcggcctgctgggctttaactgacgctgaggctcgaaagcgtggggagcaaacaggattagataccctggtagtccacgccgtaaacgatgattactaggtgtggggggactgaccccttccgtgccgcagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatcggatgcatagcctggagacaggtgaagcccttcggggcatccagacaggtggtgcatggttgtcgtcagctcgtgtcgtgagatgttgggttaagtcccgcaacgagcgcaacccttattattagttgctacgcaagagcactctaatgagactgccgttgacaaaacggaggaaggtggggatgacgtcaaatcatcatgccccttatgacctgggctacacacgtactacaatggcactaaaacagagggcggcgacaccgcgaggtgaagcgaatcccgaaaaagtgtctcagttcagattgcaggctgcaacccgcctgcatgaagtcggaattgctagtaatcgcggatcagcatgccgcggtgaatacgttcccgggccttgtacacaccgcccgtcacaccatgggagtcggtaacacccgaagccagtagcctaaccgcaaggggggcgctgtcgaaggtgggattgatgactggggtgaagtcgtaacaaggtagccgtatcggaaggtgcggctggatcacctccttt

SEQ ID NO: 5 - DNA, 1529 bp, Coprococcus sp.
  source 1..1529: mol_type = other DNA
aacgagagtttgatcctggctcaggatgaacgctggcggcgtgcctaacacatgcaagtcgaacgaagcacttacttccaaatcttcggaagaggaggtacttgactgagtggcggacgggtgagtaacgcgtggggaacctgccccgtaccgggggataacagtcagaaatgactgctaataccgcataagcgcacgaaggcgcatgcttttgtgtgaaaaactccggtggtacgggatggtcccgcgtctgattagccagttggcggggtaacggcccaccaaagcgacgatcagtagccggcctgagagggtggacggccacattgggactgagacacggcccagactcctacgggaggcagcagtggggaatattgcacaatgggggaaaccctgatgcagcgacgccgcgtgagcgaagaagtatttcggtatgtaaagctctgtcagcagggaagaaaatgacggtacctgaccaagaagcaccggctaaatacgtgccagcagccgcggtaatacgtatggtgcaagcgttatccggatttactgggtgtaaagggagcgtagacggaggggcaagtctgaagtgaaagcccggggcccaaccccgggactgctttggaaactgtccgtctggagtgccggagaggtaagcggaattcccagtgtagcggtgaaatgcgtagatattgggaggaacaccagtggcgaaggcggcttactggacggtcactgacgttgaggctcgaaagcgtggggagcaaacaggattagataccctggtagtccacgccgtaaacgatgactactaggtgtcgggtggcagagccattcggtgccgcagccaacgcagtaagtagtccacctggggagtacgttcgcaagaatgaaactcaaaggaattgacggggacccgcacaagcggtggagcatgtggtttaattcgaagcaacgcgaagaaccttacctggccttgacatccccctgaccggcgcgtaatggtgcctttccttcgggacaggggagacaggtggtgcatggttgtcgtcagctcgtgtcgtgagatgttgggttaagtcccgcaacgagcgcaacccttatcttcagtagccagcacgcagaggtgggcactctggagagactgccagggacaacctggaggaaggtggggatgacgtcaaatcatcatgccccttatggccagggctacacacgtgctacaatggcgtaaacagagggaagcgagcccgcgagggggagcaaatcccaaaaataacgtcccagttcggactgcaggctgcaacccgcctgcacgaagctggaatcgctagtaatcgcgaatcagcatgtcgcggtgaatacgttcccgggtcttgtacacaccgcccgtcacaccatgggagtcggtaacgcccgaagtcagtgacccaacctccgggagggagctgccgaaggcgggaccggtaactggggtgaagtcgtaacaaggtagccgtatcggaaggtgcggctggatcacctccttt

SEQ ID NO: 6 - DNA, 1521 bp, Anaerotruncus colihominis; identical to SEQ ID NO: 4
  source 1..1521: mol_type = other DNA

SEQ ID NO: 7 - DNA, 1425 bp, Clostridium lactatifermentans
  source 1..1425: mol_type = other DNA
cttagtggcggacgggtgagtaacgtgtgggcaacctgccctgtactggggaataatcattggaaacgatgactaataccgcatgtggtcctcggaaggcatcttctgaggaagaaaggatttattcggtacaggatgggcccgcatctgattagctagttggtgagataacagcccaccaaggcgacgatcagtagccgacctgagagggtgatcggccacattgggactgagacacggcccaaactcctacgggaggcagcagtggggaatattgcacaatgggcgaaagcctgatgcagcaacgccgcgtgaaggatgaagggtttcggctcgtaaacttctatcaatagggaagaaagaaatgacggtacctaaataagaagccccggctaactacgtgccagcagccgcggtaatacgtagggggcaagcgttatccggaattactgggtgtaaagggagcgtaggcggcatggtaagccagatgtgaaagccttgggcttaacccgaggattgcatttggaactatcaagctagagtacaggagaggaaagcggaattcctagtgtagcggtgaaatgcgtagatattaggaagaacaccagtggcgaaggcggctttctggactgaaactgacgctgaggctcgaaagcgtggggagcaaacaggattagataccctggtagtccacgccgtaaacgatgagtgctaggtgtcggggaggaatcctcggtgccgcagctaacgcaataagcactccacctggggagtacgaccgcaaggttgaaactcaaaggaattgacgggggcccgcacaagcggtggagcatgtggtttaattcgaagcaacgcgaagaaccttaccaaggcttgacatcccgatgaccgctctagagatagagcttctcttcggagcatcggtgacaggtggtgcatggttgtcgtcagctcgtgtcgtgagatgttgggttaagtcccgcaacgagcgcaacccttatcttcagtagccatcatttagttgggcactctggagagactgccgtggataacacggaggaaggtggggatgacgtcaaatcatcatgccccttatgtcttgggctacacacgtgctacaatggctggtaacaaagtgacgcaaaacggcgacgttaagcaaatcacaaaaacccagtcccagttcggattgtagtctgcaactcgactacatgaagctggaatcgctagtaatcgcgaatcagcatgtcgcggtgaatacgttcccgggccttgtacacaccgcccgtcacaccatgggagttggaagcacccgaagtcggtgacctaaccgtaaggaaggagccgccgaaggtgaagccagtgactggggtgaagtcgtaacaaggtagccgtatcggaaggtgcggctggatcacctccttt

SEQ ID NO: 8 - DNA, 1529 bp, Pseudoflavonifractor capillosus
  source 1..1529: mol_type = other DNA
tattgagagtttgatcctggctcaggatgaacgctggcggcgtgcttaacacatgcaagtcgaacggagagctaatgacagaggattcgtccaatggatttagtttcttagtggcggacgggtgagtaacgcgtgaggaacctgcctcggagtggggaataacacaacgaaagctgtgctaataccgcatgatgcagctgggtcgcatgactctggctgccaaagatttatcgctctgagatggcctcgcgtctgattagctggttggcggggtaacggcccaccaaggcgacgatcagtagccggactgagaggttggccggccacattgggactgagacacggcccagactcctacgggaggcagcagtggggaatattgggcaatgggcgcaagcctgacccagcaacgccgcgtgaaggaagaaggccctcgggttgtaaacttcttttgtcagggacgaagcaagtgacggtacctgacgaataagccacggctaactacgtgccagcagccgcggtaatacgtaggtggcaagcgttatccggatttactgggtgtaaagggcgtgtaggcgggattgcaagtcagatgtgaaaaccacgggctcaacctgtggcctgcatttgaaactgcagttcttgagtactggagaggcagacggaattcctagtgtagcggtgaaatgcgtagatattaggaggaacaccagtggcgaaggcggtctgctggacagcaactgacgctgaggcgcgaaagcgtggggagcaaacaggattagataccctggtagtccacgctgtaaacgatggatactaggtgtggggggtctgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatcccgatgaccggactagagatagtcttttctcttcggagacatcggtgacaggtggtgcatggttgtcgtcagctcgtgtcgtgagatgttgggttaagtcccgcaacgagcgcaacccctattgttagttgctacgcaagagcactctagcgagactgccgttgacaaaacggaggaaggcggggacgacgtcaaatcatcatgccccttatgtcctgggccacacacgtactacaatggtggttaacagagggaggcaataccgcgaggtggagcaaacccctaaaagccatcccagttcggattgcaggctgcaacccgcctgcatgaagttggaatcgctagtaatcgcggatcagcatgccgcggtgaatacgttcccgggccttgtacacaccgcccgtcacaccatgagagtcgggaacacccgaagtccgtagcctaaccgcaaggggggcgcggccgaaggtgggttcgataattggggtgaagtcgtaacaaggtagccgtatcggaaggtgcggctggatcacctccttt

SEQ ID NO: 9 - DNA, 1425 bp, Clostridium lactatifermentans; identical to SEQ ID NO: 7
  source 1..1425: mol_type = other DNA

SEQ ID NO: 10 - DNA, 1559 bp, Lactobacillus salivarius
  source 1..1559: mol_type = other DNA
aatgagagtttgatcctggctcaggacgaacgctggcggcgtgcctaatacatgcaagtcgaacgaaactttcttacaccgaatgcttgcattcaccgtaagaagttgagtggcggacgggtgagtaacacgtgggtaacctgcctaaaagaaggggataacacttggaaacaggtgctaataccgtatatctctaaggatcgcatgatccttagatgaaagatggttctgctatcgcttttagatggacccgcggcgtattaactagttggtggggtaacggcctaccaaggtgatgatacgtagccgaactgagaggttgatcggccacattgggactgagacacggcccaaactcctacgggaggcagcagtagggaatcttccacaatggacgcaagtctgatggagcaacgccgcgtgagtgaagaaggtcttcggatcgtaaaactctgttgttagagaagaacacgagtgagagtaactgttcattcgatgacggtatctaaccagcaagtcacggctaactacgtgccagcagccgcggtaatacgtaggtggcaagcgttgtccggatttattgggcgtaaagggaacgcaggcggtcttttaagtctgatgtgaaagccttcggcttaaccggagtagtgcattggaaactggaagacttgagtgcagaagaggagagtggaactccatgtgtagcggtgaaatgcgtagatatatggaagaacaccagtggcgaaagcggctctctggtctgtaactgacgctgaggttcgaaagcgtgggtagcaaacaggattagataccctggtagtccacgccgtaaacgatgaatgctaggtgttggagggtttccgcccttcagtgccgcagctaacgcaataagcattccgcctggggagtacgaccgcaaggttgaaactcaaaggaattgacgggggcccgcacaagcggtggagcatgtggtttaattcgaagcaacgcgaagaaccttaccaggtcttgacatcctttgaccacctaagagattaggttttcccttcggggacaaagtgacaggtggtgcatggctgtcgtcagctcgtgtcgtgagatgttgggttaagtcccgcaacgagcgcaacccttgttgtcagttgccagcattaagttgggcactctggcgagactgccggtgacaaaccggaggaaggtggggacgacgtcaagtcatcatgccccttatgacctgggctacacacgtgctacaatggacggtacaacgagtcgcaagaccgcgaggtttagctaatctcttaaagccgttctcagttcggattgtaggctgcaactcgcctacatgaagtcggaatcgctagtaatcgcgaatcagcatgtcgcggtgaatacgttcccgggccttgtacacaccgcccgtcacaccatgagagtttgtaacacccaaagccggtggggtaaccgcaaggagccagccgtctaaggtgggacagatgattggggtgaagtcgtaacaaggtagccgtaggagaacctgcggctggatcacctccttt

SEQ ID NO: 11 - DNA, 1065 bp, Lactobacillus reuteri
  source 1..1065: mol_type = other DNA
gtgacggtatccaaccagaaagtcacggctaactacgtgccagcagccgcggtaatacgtaggtggcaagcgttatccggatttattgggcgtaaagcgagcgcaggcggttgcttaggtctgatgtgaaagccttcggcttaaccgaagaagtgcatcggaaaccgggcgacttgagtgcagaagaggacagtggaactccatgtgtagcggtggaatgcgtagatatatggaagaacaccagtggcgaaggcggctgtctggtctgcaactgacgctgaggctcgaaagcatgggtagcgaacaggattagataccctggtagtccatgccgtaaacgatgagtgctaggtgttggagggtttccgcccttcagtgccggagctaacgcattaagcactccgcctggggagtacgaccgcaaggttgaaactcaaaggaattgacgggggcccgcacaagcggtggagcatgtggtttaattcgaagctacgcgaagaaccttaccaggtcttgacatcttgcgctaaccttagagataaggcgttcccttcggggacgcaatgacaggtggtgcatggtcgtcgtcagctcgtgtcgtgagatgttgggttaagtcccgcaacgagcgcaacccttgttactagttgccagcattaagttgggcactctagtgagactgccggtgacaaaccggaggaaggtggggacgacgtcagatcatcatgccccttatgacctgggctacacacgtgctacaatggacggtacaacgagtcgcaagctcgcgagagtaagctaatctcttaaagccgttctcagttcggactgtaggctgcaactcgcctacacgaagtcggaatcgctagtaatcgcggatcagcatgccgcggtgaatacgttcccgggccttgtacacaccgcccgtcacaccatgggagtttgtaacgcccaaagtcggtggcctaacctttatggagggagccgcctaaggcgggacagatgactggggtgaagtcgtaacaaggtagccgtaggagaacctgcggctggatcacctccttt

SEQ ID NO: 12 - DNA, 52 bp, synthetic construct
  misc_feature 1..52: note = primer
  source 1..52: mol_type = other DNA
tcgtcggcagcgtcagatgtgtataagagacaggtgccagcmgccgcggtaa

SEQ ID NO: 13 - DNA, 54 bp, synthetic construct
  misc_feature 1..54: note = primer
  source 1..54: mol_type = other DNA
gtctcgtgggctcggagatgtgtataagagacagggactachvgggtwtctaat

SEQ ID NO: 14 - DNA, 18 bp, synthetic construct
  misc_feature 1..18: note = primer
  source 1..18: mol_type = other DNA
cggtgaatacgttcycgg

SEQ ID NO: 15 - DNA, 19 bp, synthetic construct
  misc_feature 1..19: note = primer
  source 1..19: mol_type = other DNA
ggwtaccttgttacgactt

SEQ ID NO: 16 - DNA, 19 bp, synthetic construct
  misc_feature 1..19: note = primer
  source 1..19: mol_type = other DNA
cttgtacacaccgcccgtc

SEQ ID NO: 17 - DNA, 20 bp, synthetic construct
  misc_feature 1..20: note = primer
  source 1..20: mol_type = other DNA
cgcataacgttgaaagatgg

SEQ ID NO: 18 - DNA, 19 bp, synthetic construct
  misc_feature 1..19: note = primer
  source 1..19: mol_type = other DNA
ccttggtaggccgttaccc

SEQ ID NO: 19 - DNA, 26 bp, synthetic construct
  misc_feature 1..26: note = primer
  source 1..26: mol_type = other DNA
tcatcattcaaccaaaggagcaatcc

SEQ ID NO: 20 - DNA, 23 bp, synthetic construct
  misc_feature 1..23: note = primer
  source 1..23: mol_type = other DNA
tggtgctggaataaatgcttcat

SEQ ID NO: 21 - DNA, 26 bp, synthetic construct
  misc_feature 1..26: note = primer
  source 1..26: mol_type = other DNA
tgcatcatcttttctttgaattgttc

SEQ ID NO: 22 - DNA, 22 bp, synthetic construct
  misc_feature 1..22: note = primer
  source 1..22: mol_type = other DNA
atactataagctatgaacaacc